(19) United States
(12) Patent Application Publication
(10) Pub. No.: US 2023/0248771 A1
(43) Pub. Date: Aug. 10, 2023

(54) PLATELETS LOADED WITH ANTI-CANCER AGENTS

(71) Applicant: Cellphire, Inc., Rockville, MD (US)

(72) Inventors: Keith Andrew MOSKOWITZ, Westfield, IN (US); Rafael JORDA, Merignac (FR); Ying Yi ZHENG, Rockville, MD (US); Daniel Allen Sheik, West Lafayette, IN (US)

(73) Assignee: Cellphire, Inc., Rockville, MD (US)

(21) Appl. No.: 18/297,581

(22) Filed: Apr. 7, 2023

Related U.S. Application Data
(63) Continuation-in-part of application No. 17/104,528, filed on Nov. 25, 2020.
Application No. 17/104,528 is a continuation-in-part of application No. 16/698,645, filed on Nov. 27, 2019.
Application No. 16/698,645 is a continuation-in-part of application No. 16/698,583, filed on Nov. 27, 2019.
Application No. 16/698,583 is a continuation-in-part of application No. 16/697,401, filed on Nov. 27, 2019.
(60) Provisional application No. 62/940,995, filed on Nov. 27, 2019.
(60) Provisional application No. 62/828,041, filed on Apr. 2, 2019.
(60) Provisional application No. 62/775,141, filed on Dec. 4, 2018.
(60) Provisional application No. 62/773,931, filed on Nov. 30, 2018.
(60) Provisional application No. 62/828,041, filed on Apr. 2, 2019.
(60) Provisional application No. 62/775,141, filed on Dec. 4, 2018.
(60) Provisional application No. 62/773,931, filed on Nov. 30, 2018.
(60) Provisional application No. 62/828,043, filed on Apr. 2, 2019.
(60) Provisional application No. 62/775,141, filed on Dec. 4, 2018.
(60) Provisional application No. 62/773,931, filed on Nov. 30, 2018.

(51) Int. Cl. A61K 35/19; A61K 47/22; A61K 31/704; A61K 31/337; A61K 31/502; A61K 38/14; A61K 49/00; A61K 38/16; A61K 31/7105; A61K 9/19

(52) U.S. Cl. CPC A61K 35/19; A61K 47/22; A61K 31/704; A61K 31/337; A61K 31/502; A61K 38/14; A61K 49/0052; A61K 38/164; A61K 31/7105; A61K 9/19

(57) ABSTRACT

In some embodiments provided herein is a method of preparing cargo-loaded platelets, comprising: treating platelets with a cargo and with a loading buffer comprising a salt, a base, a loading agent, and optionally ethanol, to form the cargo-loaded platelets.

### CROSS-REFERENCE TO RELATED APPLICATIONS

This application is a Continuation-In-Part of U.S. patent application Ser. No. 16/697,401, filed on Nov. 27, 2019, U.S. patent application Ser. No. 16/698,583, filed on Nov. 27, 2019, U.S. patent application Ser. No. 16/698,645, filed on Nov. 27, 2019, and U.S. patent application Ser. No. 17/104,528, filed on Nov. 25, 2020. U.S. patent application Ser. No. 16/697,401 claims benefit of U.S. Provisional Patent Application No. 62/773,931, filed on Nov. 30, 2018, U.S. Provisional Patent Application No. 62/775,141, filed on Dec. 4, 2018, and U.S. Provisional Patent Application No. 62/828,043, filed on Apr. 2, 2019. U.S. patent application Ser. No. 16/698,583 claims benefit of U.S. Provisional Patent Application No. 62/828,041, filed on Apr. 2, 2019, U.S. Provisional Patent Application No. 62/775,141, filed on Dec. 4, 2018, and U.S. Provisional Patent Application No. 62/773,931, filed on Nov. 30, 2018. U.S. patent application Ser. No. 16/698,645 claims benefit of U.S. Provisional Patent Application No. 62/773,931, filed on Nov. 30, 2018, U.S. Provisional Patent Application No. 62/775,141, filed on Dec. 4, 2018, and U.S. Provisional Patent Application No. 62/828,041, filed on Apr. 2, 2019. U.S. patent application Ser. No. 17/104,528 claims benefit of U.S. Provisional Patent Application No. 62/940,995, filed on Nov. 27, 2019. The content of each of the applications listed above is incorporated herein by reference in its entirety.

### TECHNICAL FIELD

The present disclosure in some embodiments relates to the use of platelets, platelet derivatives, or thrombosomes (e.g., freeze-dried platelet derivatives) as biological carriers of cargo, such as pharmaceutical drugs, also referred to herein as drug-loaded platelets, platelet derivatives, or thrombosomes. Also provided herein in some embodiments are methods of preparing platelets, platelet derivatives, or thrombosomes loaded with the drug of interest.

The present disclosure relates to the field of blood and blood products. More specifically, it relates to platelets, cryopreserved platelets, and/or lyopreserved platelet compositions, including those containing stabilized platelets or compositions derived from platelets. The drug-loaded platelets can be stored under typical ambient conditions, refrigerated, cryopreserved, for example with dimethyl sulfoxide (DMSO), and/or lyophilized after stabilization (e.g., thrombosomes)

### BACKGROUND

Blood is a complex mixture of numerous components. In general, blood can be described as comprising four main parts: red blood cells, white blood cells, platelets, and plasma. The first three are cellular or cell-like components, whereas the fourth (plasma) is a liquid component comprising a wide and variable mixture of salts, proteins, and other factors necessary for numerous bodily functions. The components of blood can be separated from each other by various methods. In general, differential centrifugation is most commonly used currently to separate the different components of blood based on size and, in some applications, density.

Unactivated platelets, which are also commonly referred to as thrombocytes, are small, often irregularly-shaped (e.g., discoidal or ovoidal) megakaryocyte-derived components of blood that are involved in the clotting process. They aid in protecting the body from excessive blood loss due not only to trauma or injury, but to normal physiological activity as well. Platelets are considered crucial in normal hemostasis, providing the first line of defense against blood escaping from injured blood vessels. Platelets generally function by adhering to the lining of broken blood vessels, in the process becoming activated, changing to an amorphous shape, and interacting with components of the clotting system that are present in plasma or are released by the platelets themselves or other components of the blood. Purified platelets have found use in treating subjects with low platelet count (thrombocytopenia) and abnormal platelet function (thrombasthenia). Concentrated platelets are often used to control bleeding after injury or during acquired platelet function defects or deficiencies, for example those occurring during surgery and those due to the presence of platelet inhibitors.

Loading platelets with pharmaceutical drugs may allow targeted delivery of the drugs to sites of interest. Further, drug-loaded platelets may be lyophilized or cryopreserved to allow for long-term storage. In some embodiments the loading of a drug in the platelets mitigates systemic side effects associated with the drug and lowers the threshold of therapeutic dose necessary by facilitating targeted treatment at site of interest. See, Xu P., et. al., Doxorubicin-loaded platelets as a smart drug delivery system: An improved therapy for lymphoma, Scientific Reports, 7, Article Number: 42632, (2017), which is incorporated by reference herein in its entirety.

### SUMMARY OF THE INVENTION

In some embodiments provided herein is a method of preparing cargo-loaded platelets, cargo-loaded platelet derivatives, or cargo-loaded thrombosomes (e.g., freeze-dried platelet derivatives), comprising:
treating platelets, platelet derivatives, or thrombosomes with a cargo and with at least one loading agent and optionally one or more plasticizers such as organic solvents, such as organic solvents selected from the group consisting of ethanol, acetic acid, acetone, acetonitrile, dimethylformamide, dimethyl sulfoxide, dioxane, methanol, n-propanol, isopropanol, tetrahydrofuran (THF), N-methyl pyrrolidone, dimethylacetamide (DMAC), or combinations thereof,
to form the cargo-loaded platelets, cargo-loaded platelet derivatives, or cargo-loaded thrombosomes.

In some embodiments, the method of preparing cargo-loaded platelets can include treating the platelets, the platelet derivatives, and/or the thrombosomes with the cargo with one loading agent. In some embodiments, the method of preparing cargo-loaded platelets, cargo-loaded platelet derivatives, or cargo-loaded thrombosomes can include treating the platelets, the platelet derivatives, or the thrombosomes with the cargo with multiple loading agents.

In some embodiments, suitable organic solvents include, but are not limited to alcohols, esters, ketones, ethers, halogenated solvents, hydrocarbons, nitriles, glycols, alkyl nitrates, water or mixtures thereof. In some embodiments, suitable organic solvents includes, but are not limited to methanol, ethanol, n-propanol, isopropanol, acetic acid, acetone, methyl ethyl ketone, methyl isobutyl ketone, methyl acetate, ethyl acetate, isopropyl acetate, tetrahydrofuran, isopropyl ether (IPE), tert-butyl methyl ether, dioxane (e.g., 1,4-dioxane), acetonitrile, propionitrile, methylene chloride, chloroform, toluene, anisole, cyclohexane, hexane, heptane, ethylene glycol, nitromethane, dimethylformamide, dimethyl sulfoxide, N-methyl pyrrolidone, dimethylacetamide, and combinations thereof. The presence of organic solvents, such as ethanol, can be beneficial in the processing of platelets, platelet derivatives, and/or thrombosomes. In particular, the organic solvent may open up and/or increase the flexibility of the plasma membrane of the platelets, platelet derivatives, and/or thrombosomes, which allows a higher amount of cargo (e.g., drug) to be loaded into the platelets, platelet derivatives, and/or thrombosomes. In some embodiments, the organic solvent can aid in solubilizing molecules to be loaded.

In some embodiments provided herein is a method of preparing drug-loaded platelets, drug-loaded platelet derivatives, or drug-loaded thrombosomes, comprising:
treating platelets, platelet derivatives, or thrombosomes with a drug and with a loading buffer comprising a base, a loading agent, and optionally at least one organic solvent such as an organic solvent selected from the group consisting of ethanol, acetic acid, acetone, acetonitrile, dimethylformamide, dimethyl sulfoxide, dioxane, methanol, n-propanol, isopropanol, tetrahydrofuran (THF), N-methyl pyrrolidone, dimethylacetamide (DMAC), or combinations thereof,
to form the drug-loaded platelets, the drug-loaded platelet derivatives, or the drug-loaded thrombosomes.

In some embodiments provided herein is a method of preparing cargo-loaded platelets, cargo-loaded platelet derivatives, or cargo-loaded thrombosomes, comprising: treating platelets, platelet derivatives, or thrombosomes with a cargo and with a loading buffer comprising a salt, a base, a loading agent, and optionally at least one organic solvent
to form the cargo-loaded platelets, cargo-loaded platelet derivatives, or the cargo-loaded thrombosomes.

In some embodiments provided herein is a method of preparing cargo-loaded platelets, cargo-loaded platelet derivatives, or cargo-loaded thrombosomes, comprising:
treating platelets, platelet derivatives, or thrombosomes with a cargo and with a loading agent and optionally at least one organic solvent
to form the cargo-loaded platelets, the cargo-loaded platelet derivatives, or the cargo-loaded thrombosomes.

In some embodiments provided herein is a method of preparing drug-loaded platelets, drug-loaded platelet derivatives, or drug-loaded thrombosomes, comprising:
treating platelets, platelet derivatives, or thrombosomes with a drug and with a loading buffer comprising a base, a loading agent, and optionally at least one organic solvent
to form the drug-loaded platelets, the drug-loaded platelet derivatives, or the drug-loaded thrombosomes.

In some embodiments provided herein is a method of preparing drug-loaded platelets, drug-loaded platelet derivatives, or drug-loaded thrombosomes, comprising:
treating platelets, platelet derivatives, or thrombosomes with a drug and with a loading buffer comprising a salt, a base, a loading agent, and optionally at least one organic solvent
to form the drug-loaded platelets, the drug-loaded platelet derivatives, or the drug-loaded thrombosomes.

In some embodiments provided herein is a method of preparing drug-loaded platelets, drug-loaded platelet derivatives, or drug-loaded thrombosomes, comprising:
a) providing platelets, platelet derivatives, or thrombosomes;
   and
b) treating the platelets, the platelet derivatives, or the thrombosomes with a drug and with a loading buffer comprising a salt, a base, a loading agent, and optionally at least one organic solvent
   to form the drug-loaded platelets, drug-loaded platelet derivatives, or the drug-loaded thrombosomes.

In some embodiments, the method further comprises cryopreserving the drug-loaded platelets, drug-loaded platelet derivatives, or the drug-loaded thrombosomes. In some embodiments, the method further comprises cold storing the drug-loaded platelets, drug-loaded platelet derivatives, or the drug-loaded thrombosomes. In some embodiments, the method further comprises drying the drug-loaded platelets or the drug-loaded platelet derivatives. In some embodiments, the method further comprises freeze-drying the drug-loaded platelets or the drug-loaded platelet derivatives. In such embodiments, the method may further comprise rehydrating the drug-loaded platelets, drug-loaded platelet derivatives, or drug-loaded thrombosomes obtained from the drying step.

In some embodiments, the method that further comprises drying the drug-loaded platelets or drug-loaded platelet derivatives and rehydrating the drug-loaded platelets or the drug-loaded platelet derivatives obtained from the drying step provides rehydrated platelets or platelet derivatives comprising at least 10% of the amount of the drug of step (b).

In some embodiments, the method that further comprises drying the drug-loaded platelets or the drug-loaded platelet derivatives and rehydrating the drug-loaded platelets or the drug-loaded platelet derivatives obtained from the drying step provides rehydrated platelets or platelet derivatives comprising from about 1 nM to about 100 mM, such as about 10 nM to about 10 mM, such as about 100 nM to 1 mM, of the drug.

In some embodiments, the platelets, platelet derivatives, or thrombosomes are treated with the drug and with the buffer sequentially, in either order.

Thus, in some embodiments provided herein is a method of preparing drug-loaded platelets, drug-loaded platelet derivatives, or drug-loaded thrombosomes, comprising:
(1) treating platelets, platelet derivatives, or thrombosomes with a drug to form a first composition; and
(2) treating the first composition with a buffer comprising a salt, a base, a loading agent, and optionally at least one organic solvent to form the drug-loaded platelets, drug-loaded platelet derivatives, or drug-loaded thrombosomes.

In some embodiments of the methods of preparing cargo-loaded platelets, such as drug-loaded platelets, as provided herein, the methods do not comprise treating platelets, platelet derivatives, or thrombosomes with ethanol.

In some embodiments of the methods of preparing cargo-loaded platelets, such as drug-loaded platelets, as provided herein, the methods do not comprise treating platelets, platelet derivatives, or thrombosomes with a solvent selected from the group consisting of ethanol, acetic acid, acetone, acetonitrile, dimethylformamide, dimethyl sulfoxide, dioxane, methanol, n-propanol, isopropanol, tetrahydrofuran (THF), N-methyl pyrrolidone, dimethylacetamide (DMAC), or combinations thereof.

In some embodiments of the methods of preparing cargo-loaded platelets, such as drug-loaded platelets, as provided herein, the methods do not comprise treating platelets, platelet derivatives, or thrombosomes with an organic solvent.

In some embodiments of the methods of preparing cargo-loaded platelets, such as drug-loaded platelets, as provided herein, the methods do not comprise treating platelets, platelet derivatives, or thrombosomes with a solvent.

In some embodiments of the methods of preparing cargo-loaded platelets, such as drug-loaded platelets, as provided herein, the methods comprise treating platelets, platelet derivatives, or thrombosomes with a solvent, such as an organic solvent, such as organic solvent selected from the group consisting of ethanol, acetic acid, acetone, acetonitrile, dimethylformamide, dimethyl sulfoxide, dioxane, methanol, n-propanol, isopropanol, tetrahydrofuran (THF), N-methyl pyrrolidone, dimethylacetamide (DMAC), or combinations thereof, such as ethanol.

In some embodiments, the method further comprises drying the drug-loaded platelets or the drug-loaded platelet derivatives obtained in step (2). In some embodiments, the method further comprises cryopreserving, lyopreserving (e.g., freeze-drying) the drug-loaded platelets or the drug-loaded platelet derivatives. In some embodiments, the method further comprises cold storing the drug-loaded platelets, the drug-loaded platelet derivatives, the drug-loaded thrombosomes, or compositions containing drug-loaded platelets at suitable storage temperatures, such as standard room temperature storing (e.g., storing at a temperature ranging from about 20 to about 30° C.) or cold storing (e.g., storing at a temperature ranging from about 1 to about 10° C.). In some embodiments, the method further comprises cryopreserving, freeze-drying, thawing, rehydrating, and combinations thereof, the drug loaded platelets, the drug-loaded platelet derivatives, or the drug-loaded thrombosomes. For example, in such embodiments, the method may further comprise rehydrating the drug-loaded platelets, the drug-loaded platelet derivatives, or the drug-loaded thrombosomes obtained from the drying step.

In some embodiments, the method that further comprises drying the drug-loaded platelets or drug-loaded platelet derivatives and rehydrating the drug-loaded platelets or the drug-loaded platelet derivatives obtained from the drying step provides rehydrated platelets or platelet derivatives comprising at least 10% of the amount of the drug of step (1).

In some embodiments, the method that further comprises drying the drug-loaded platelets or the drug-loaded platelet derivatives and rehydrating the drug-loaded platelets or the drug-loaded platelet derivatives obtained from the drying step provides rehydrated platelets or platelet derivatives comprising from about 1 nM to about 1000 mM, such as about 10 nM to about 100 mM, such as about 100 nM to 10 mM, of the drug of step (1).

In some embodiments provided herein is a method of preparing drug-loaded platelets, drug-loaded platelet derivatives, or drug-loaded thrombosomes, comprising:
(1) treating the platelets, platelet derivatives, or thrombosomes with a buffer comprising a salt, a base, a loading agent, and optionally ethanol, to form a first composition; and
(2) treating the first composition with a drug, to form the drug-loaded platelets, the drug-loaded platelet derivatives, or the drug-loaded thrombosomes.

In some embodiments, the method further comprises drying the drug-loaded platelets, the drug-loaded platelet derivatives, or the drug-loaded thrombosomes obtained in step (2). In some embodiments, the method further comprises freeze-drying the drug-loaded platelets or the drug-loaded platelet derivatives. In such embodiments, the method may further comprise rehydrating the drug-loaded platelets or the drug-loaded platelet derivatives obtained from the drying step.

In some embodiments, the method that further comprises drying the drug-loaded platelets or the drug-loaded platelet derivatives and rehydrating the drug-loaded platelets or the drug-loaded platelet derivatives obtained from the drying step provides rehydrated platelets or platelet derivatives comprising at least 10% of the amount of the drug of step (2).

In some embodiments, the method that further comprises drying the drug-loaded platelets or the drug-loaded platelet derivatives and rehydrating the drug-loaded platelets or the drug-loaded platelet derivatives obtained from the drying step provides rehydrated platelets or thrombosomes comprising from about 1 nM to about 1000 mM, such as about 10 nM to about 10 mM, such as about 100 nM to 1 mM, of the drug of step (2).

In some embodiments, the platelets or thrombosomes are treated with the drug and with the buffer concurrently.

Thus, in some embodiments provided herein is a method of preparing drug-loaded platelets, the drug-loaded platelet derivatives, or the drug-loaded thrombosomes, comprising: treating the platelets, the platelet derivatives, or the thrombosomes with a drug in the presence of a buffer comprising a salt, a base, a loading agent, and optionally ethanol, to form the drug-loaded platelets, the drug-loaded platelet derivatives, or the drug-loaded thrombosomes.

In some embodiments, the method further comprises drying the drug-loaded platelets or the drug-loaded platelet derivatives. In some embodiments, the method further comprises freeze-drying the drug-loaded platelets or the drug-loaded platelet derivatives. In such embodiments, the method may further comprise rehydrating the drug-loaded platelets or the drug-loaded platelet derivatives obtained from the drying step.

In some embodiments, the method that further comprises drying the drug-loaded platelets or the drug-loaded platelet derivatives and rehydrating the drug-loaded platelets or the drug-loaded platelet derivatives obtained from the drying step provides rehydrated platelets or the thrombosomes comprising at least 10% of the amount of the drug prior to loading.

In some embodiments, the method that further comprises drying the drug-loaded platelets or the drug-loaded platelet derivatives and rehydrating the drug-loaded platelets or the drug-loaded platelet derivatives obtained from the drying step provides rehydrated platelets or thrombosomes comprising from about 1 nM to about 1000 mM, such as about 10 nM to about 10 mM, such as about 100 nM to 1 mM, of the drug.

In some embodiments, platelets, platelet derivatives, or thrombosomes are pooled from a plurality of donors. Such platelets, platelet derivatives, and thrombosomes pooled from a plurality of donors may be also referred herein to as pooled platelets, platelet derivatives, or thrombosomes. In some embodiments, the donors are more than 5, such as more than 10, such as more than 20, such as more than 50, such as up to about 100 donors. In some embodiments, the donors are from about 5 to about 100, such as from about 10 to about 50, such as from about 20 to about 40, such as from about 25 to about 35.

Thus, provided herein in some embodiments is a method of preparing drug-loaded platelets, drug-loaded platelet derivatives, or drug-loaded thrombosomes comprising
A) pooling platelets, platelet derivatives, or thrombosomes from a plurality of donors; and
B) treating the platelets, platelet derivatives, or thrombosomes from step (A) with a drug and with a loading buffer comprising a salt, a base, a loading agent, and optionally ethanol, to form the drug-loaded platelets, the drug-loaded platelet derivatives, or the drug-loaded thrombosomes.

In some embodiments, the method further comprises drying the drug-loaded platelets or the drug-loaded platelet derivatives obtained in step (B). In some embodiments, the method further comprises freeze-drying the drug-loaded platelets or the drug-loaded platelet derivatives. In such embodiments, the method may further comprise rehydrating the drug-loaded platelets or the drug-loaded platelet derivatives obtained from the drying step.

In some embodiments, the method that further comprises drying the drug-loaded platelets or the drug-loaded platelet derivatives and rehydrating the drug-loaded platelets or the drug-loaded platelet derivatives obtained from the drying step provides rehydrated platelets or rehydrated platelet derivatives comprising at least 10% of the amount of the drug of step (B).

In some embodiments, the method that further comprises drying the drug-loaded platelets or the drug-loaded platelet derivatives and rehydrating the drug-loaded platelets or the drug-loaded platelet derivatives obtained from the drying step provides rehydrated platelets or rehydrated platelet derivatives comprising from about 1 nM to about 1000 mM, such as about 10 nM to about 10 mM, such as about 100 nM to 1 mM, of the drug of step (B).

In some embodiments, the pooled platelets, platelet derivatives, or thrombosomes are treated with the drug and with the buffer sequentially, in either order.

Thus, provided herein in some embodiments is a method of preparing drug-loaded platelets, drug-loaded platelet derivatives, or drug-loaded thrombosomes comprising:
A) pooling platelets, platelet derivatives, or thrombosomes from a plurality of donors; and
B)
   (1) treating the platelets, platelet derivatives, or thrombosomes from step (A) with a drug to form a first composition; and
   (2) treating the first composition with a buffer comprising a salt, a base, a loading agent, and optionally ethanol, to form the drug-loaded platelets, drug-loaded platelet derivatives, or drug-loaded thrombosomes.

In some embodiments, the method further comprises drying the drug-loaded platelets or the drug-loaded platelet derivatives obtained in step (B)(2). In some embodiments, the method further comprises freeze-drying the drug-loaded platelets or the drug-loaded platelet derivatives. In such embodiments, the method may further comprise rehydrating the drug-loaded platelets or the drug-loaded platelet derivatives obtained from the drying step.

In some embodiments, the method that further comprises drying the drug-loaded platelets or the drug-loaded platelet derivatives and rehydrating the drug-loaded platelets or the drug-loaded platelet derivatives obtained from the drying step provides rehydrated platelets or rehydrated platelet derivatives comprising at least 10% of the amount of the drug of step (B)(1).

In some embodiments, the method that further comprises drying the drug-loaded platelets or the drug-loaded platelet derivatives and rehydrating the drug-loaded platelets or the drug-loaded platelet derivatives obtained from the drying step provides rehydrated platelets or platelet derivatives comprising from about 1 nM to about 1000 mM, such as about 10 nM to about 10 mM, such as about 100 nM to 1 mM, of the drug of step (B)(1).

Thus, provided herein in some embodiments is a method of preparing drug-loaded platelets, drug-loaded platelet derivatives, or drug-loaded thrombosomes comprising:
A) pooling platelets, platelet derivatives, or thrombosomes from a plurality of donors; and
B)
   (1) treating the platelets, the platelet derivatives, or the thrombosomes from step (A) with a buffer comprising a salt, a base, a loading agent, and optionally ethanol, to form a first composition; and
   (2) treating the first composition with a drug to form the drug-loaded platelets, the drug-loaded platelet derivatives, or the drug-loaded thrombosomes.

In some embodiments, the method further comprises drying the drug-loaded platelets or the drug-loaded platelet derivatives obtained in step (B)(2). In some embodiments, the method further comprises freeze-drying the drug-loaded platelets or the drug-loaded platelet derivatives. In such embodiments, the method may further comprise rehydrating the drug-loaded platelets or the drug-loaded platelet derivatives obtained from the drying step.

In some embodiments, the method that further comprises drying the drug-loaded platelets or the drug-loaded platelet derivatives and rehydrating the drug-loaded platelets or the drug-loaded platelet derivatives obtained from the drying step provides rehydrated platelets or thrombosomes comprising at least 10% of the amount of the drug of step (B)(2).

In some embodiments, the method that further comprises drying the drug-loaded platelets or the drug-loaded platelet derivatives and rehydrating the drug-loaded platelets or the drug-loaded platelet derivatives obtained from the drying step provides rehydrated platelets or thrombosomes comprising from about 1 nM to about 1000 mM, such as about 10 nM to about 10 mM, such as about 100 nM to 1 mM, of the drug of step (B)(2).

In some embodiments, the pooled platelets, platelet derivatives, or thrombosomes are treated with the drug and with the buffer concurrently.

Thus, in some embodiments provided herein is a method of preparing drug-loaded platelets, drug-loaded platelet derivatives, or drug-loaded thrombosomes, comprising:
A) pooling platelets, platelet derivatives, or thrombosomes from a plurality of donors; and
B) treating the platelets, the platelet derivatives, or the thrombosomes with a drug in the presence of a buffer comprising a salt, a base, a loading agent, and optionally ethanol, to form the drug-loaded platelets, the drug-loaded platelet derivatives, or the drug-loaded thrombosomes.

In some embodiments, the method further comprises drying the drug-loaded platelets or the drug-loaded platelet derivatives obtained in step (B). In some embodiments, the method further comprises freeze-drying the drug-loaded platelets or the drug-loaded platelet derivatives. In such embodiments, the method may further comprise rehydrating the drug-loaded platelets or the drug-loaded platelet derivatives obtained from the drying step.

In some embodiments, the method that further comprises drying the drug-loaded platelets or the drug-loaded platelet derivatives and rehydrating the drug-loaded platelets or the drug-loaded platelet derivatives obtained from the drying step provides rehydrated platelets or thrombosomes comprising at least 10% of the amount of the drug of step (B).

In some embodiments, the method that further comprises drying the drug-loaded platelets or the drug-loaded platelet derivatives and rehydrating the drug-loaded platelets or the drug-loaded platelet derivatives obtained from the drying step provides rehydrated platelets or thrombosomes comprising from about 1 nM to about 1000 mM, such as about 10 nM to about 10 mM, such as about 100 nM to 1 mM, of the drug of step (B).

In some embodiments, the methods of preparing drug-loaded platelets, drug-loaded platelet derivatives, or drug-loaded thrombosomes that comprise pooling platelets, platelet derivatives, or thrombosomes from a plurality of donors comprise a viral inactivation step.

In some embodiments, the methods of preparing drug-loaded platelets, drug-loaded platelet derivatives, or drug-loaded thrombosomes that comprise pooling platelets, platelet derivatives, or thrombosomes from a plurality of donors do not comprise a viral inactivation step.

In some embodiments, the platelets, the platelet derivatives, or the thrombosomes are loaded with the drug in a period of time of 5 minutes to 48 hours, such as 10 minutes to 24 hours, such as 20 minutes to 12 hours, such as 30 minutes to 6 hours, such as 1 hour to 3 hours, such as about 2 hours. In some embodiments, a concentration of drug from about 1 nM to about 1000 mM, such as about 10 nM to about 10 mM, such as about 100 nM to 1 mM, is loaded in a period of time of 5 minutes to 48 hours, such as 10 minutes to 24 hours, such as 20 minutes to 12 hours, such as 30 minutes to 6 hours, such as 1 hour minutes to 3 hours, such as about 2 hours.

In some embodiments provided herein are drug-loaded platelets, drug-loaded platelet derivatives, or drug-loaded thrombosomes prepared by a method as disclosed herein. In some embodiments provided herein are rehydrated platelets, platelet derivatives, or thrombosomes prepared by a method as disclosed herein.

In some embodiments provided herein are drug-loaded platelets, drug-loaded platelet derivatives, or drug-loaded thrombosomes prepared with Prostaglandin E1 (PGE1) or Prostacyclin. In some embodiments provided herein are drug-loaded platelets, drug-loaded platelet derivatives, or drug-loaded thrombosomes that are not prepared with Prostaglandin E1 (PGE1) or Prostacyclin.

In some embodiments provided herein are drug-loaded platelets, drug-loaded platelet derivatives, or drug-loaded thrombosomes prepared with a chelating agent such as EGTA. In some embodiments provided herein are drug-loaded platelets, drug-loaded platelet derivatives, or drug-loaded thrombosomes that are not prepared with a chelating agent such as EGTA.

In some embodiments provided herein the method includes treating the first composition with Prostaglandin 1 (PGE1) or Prostacyclin. In some embodiments provided herein the method does not include treating the first composition with Prostaglandin 1 (PGE1) or Prostacyclin.

In some embodiments provided herein the method includes treating the first composition with a chelating agent such as EGTA. In some embodiments provided herein the method does not include treating the first composition with a chelating agent such as EGTA.

In some embodiments provided herein are drug-loaded platelets, drug-loaded platelet derivatives, or drug-loaded thrombosomes prepared with an anti-aggregation agent.

In some embodiments provided herein are drug-loaded platelets, drug-loaded platelet derivatives, or drug-loaded thrombosomes prepared with an anti-aggregation agent such as GPIIb/IIIa inhibitor. In some embodiments the GPIIb/IIIa inhibitor is GR144053. In some embodiments, GR144053 is present at a concentration of at least 1.2 μM.

In some embodiments provided herein are drug-loaded platelets, drug-loaded platelet derivatives, or drug-loaded thrombosomes prepared with an anti-aggregation agent before being treated with the drug. In some embodiments provided herein are drug-loaded platelets, drug-loaded platelet derivatives, or drug-loaded thrombosomes prepared with an anti-aggregation agents concurrently with the drug.

In some embodiments provided herein are drug-loaded platelets, drug-loaded platelet derivatives, or drug-loaded thrombosomes prepared with a drug where the drug is a drug for the treatment of cancer. In some embodiments provided herein the cancer is hemangiosarcoma.

In some embodiments provided herein the drug for the treatment of cancer is doxorubicin. In some embodiments, the drug for the treatment of hemangiosarcoma is doxorubicin.

In some embodiments provided herein the cancer includes hemangiosarcoma. In some embodiments provided herein the drug is a drug for the treatment of hemangiosarcoma includes doxorubicin.

In some embodiments provided herein the drug for the treatment of cancer is paclitaxel.

In some embodiments provided herein the drug for the treatment of cancer is a PARP inhibitor. In some embodiments provided herein the PAPR inhibitor is Olaparib.

Drug-loaded platelets, drug-loaded platelet derivatives, or drug-loaded thrombosomes may shield the drug from exposure in circulation, thereby reducing or eliminating systemic toxicity (e.g. cardiotoxicity) associated with the drug. Drug-loaded platelets, drug-loaded platelet derivatives, or drug-loaded thrombosomes may also protect the drug from metabolic degradation or inactivation. Drug delivery with drug-loaded platelets, drug-loaded platelet derivatives, or drug-loaded thrombosomes may therefore be advantageous in treatment of diseases such as cancer, since drug-loaded platelets, drug-loaded platelet derivatives, or drug-loaded thrombosomes facilitate targeting of cancer cells while mitigating systemic side effects. Drug-loaded platelets, drug-loaded platelet derivatives, or drug-loaded thrombosomes may be used in any therapeutic setting in which expedited healing process is required or advantageous. Potential applications include, for example, targeted depletion of cancer cells with chemotherapy drugs.

Accordingly, in some embodiments, provided herein is a method of treating a disease as disclosed herein, comprising administering drug-loaded platelets, drug-loaded platelet derivatives, or drug-loaded thrombosomes as disclosed herein. Accordingly, in some embodiments, provided herein is a method of treating a disease as disclosed herein, comprising administering cold stored, room temperature stored, cryopreserved thawed, rehydrated, and/or lyophilized platelets, platelet derivatives, or thrombosomes as disclosed herein. In some embodiments, the disease is cancer.

### DESCRIPTION OF DRAWINGS

FIG. 1 shows resulting amounts of doxorubicin load in platelets as a function of incubation time when evaluated by flow cytometry.

FIG. 2 shows resulting amounts of doxorubicin load in platelets following ADP and TRAP simulation when evaluated by flow cytometry.

FIG. 3 shows fluorescence intensity as measured by flow cytometry after platelet incubation with DOX encapsulated liposomes.

FIG. 4 shows flow cytometry data relating to the endocytic loading of DOX into platelets. The top graph (labeled A) shows amalgamated data that includes the bottom left graph (labeled B) (Doxorubicin at 0.1 mM) and the bottom right graph (labeled C) (Doxorubicin at 0.3 mM).

FIG. 5 provides flow cytometry data relating to liposome-mediated loading of Doxorubicin into platelets.

FIG. 6 provides a comparison of doxosome (liposome encapsulated doxorubicin) loading efficiency in platelets between conventional HMT buffer (Protocol 3, shown in continuous line) and trehalose-containing loading buffer (Protocol 4, shown in individual points). The graph on the left (labeled as A) shows platelets with CD42b antibodies, while the graph on the right (labeled as B) shows platelets without CD42b.

FIG. 7 shows the correlation between fluorescence and concentration of doxorubicin.

FIG. 8 shows the total concentration of (a) intracellular doxorubicin (triangle), and (b) membrane-bound doxorubicin (circle), with increasing concentration of platelets/μL.

FIG. 9 shows how for a given concentration of doxorubicin (DOX) (0.2 mM), the amount of DOX/platelet decreases as the number of platelets increases.

FIG. 10 shows the effect of collagen on inducing drug release from doxorubicin-loaded platelets. Released DOX is plotted on the left and intracellular DOX is plotted to the right for each group.

FIG. 11 shows the effect of TRAP-6, ADP, arachidonic acid, and thrombin on inducing drug release from doxorubicin-loaded platelets. Released DOX is plotted on the left and intracellular DOX is plotted to the right for each group.

FIG. 12 shows a comparison of the amount of doxorubicin loaded into platelets with loading buffer (right) vs. the amount loaded with HMT buffer (left).

FIG. 13A shows the effect of DOX on the aggregation of platelets as measured by light transmittance.

FIG. 13B shows the effect of the addition of a GPIIb/IIIa inhibitor, GR 144053, on the aggregation of platelets by 0 (top), 0.5 mg/mL (middle), or 1 mg/mL (bottom) doxorubicin as measured by light transmittance.

FIG. 13C shows the effect of the addition of Tirofiban on the aggregation of platelets by 0 (top) or 0.65 mg/mL (bottom) doxorubicin.

FIG. 14 shows the effect of increasing concentration of a PARP inhibitor (olaparib) on the total number of platelets loaded with PARPi and the amount of PARPi loaded.

FIG. 15 shows the total drug load (PARP inhibitor) increase following incubation of platelets with increasing concentration of drug.

FIG. 16 shows punctate intracellular staining of fluorescently tagged PARP inhibitor (80 μM) after incubation with platelets for 3 h.

FIG. 17 shows the release of DOX from within platelets in response to strong platelet activating agents after cryopreservation for up to 7 days. Day 2 is plotted on the left and Day 7 is plotted on the right for each group.

FIG. 18 shows DOX-loaded thrombosomes (mg/Plt) prepared by incubation with 600 μM Dox. Pre-lyo is during preparation before lyophilization and post-lyo is after lyophilization and rehydration of the DOX-loaded Thrombosomes.

FIG. 19 shows that the intracellular DOX concentration is increased 50-fold prior to lyophilization (platelets) and maintained at 30-fold after lyophilization (thrombosomes) relative to the external DOX concentration during incubation.

FIG. 20 shows platelet counts according to AcT-Diff hematology analyzer remain unchanged after lyophilization (thrombosomes). Unloaded Thrombosomes is plotted on the left and DOX-loaded Thrombosomes is plotted to the right for each group.

FIG. 21 shows that DOX loaded thrombosomes have similar platelet receptor biomarker expression relative to unloaded thrombosomes immediately prior to (platelets) and after lyophilization (thrombosomes).

FIG. 22 shows DOX-loaded thrombosomes adhere to collagen and tissue factor coated microchips similar to unloaded thrombosomes.

FIG. 23 shows DOX-loaded thrombosomes generate thrombin similar to unloaded thrombosomes.

FIG. 24 shows the effect of DOX-loaded thrombosomes on cell growth in a hemangiosarcoma cell model using different amounts of DOX-loaded and unloaded thrombosomes.

FIG. 25A shows that paclitaxel loads into platelets in a dose-dependent manner as measured by flow cytometry. 200 μM/4 μM is plotted on the bottom and 400 μM/8 μM is plotted on top.

FIG. 25 B shows that the percent of platelets loaded with paclitaxel increases with increasing concentration of paclitaxel over time. 200 μM/4 μM is plotted on the bottom and 400 μM/8 μM is plotted on top.

FIG. 26 shows that platelet counts remain more stable as a function of a higher initial starting cell count and minimally 50% are retained after loading. PTX in 1% DMSO is plotted on the bottom, PTX in 5% DMSO is plotted in the middle and unloaded is plotted on top. FIG. 26 A, FIG. 26 B, and FIG. 26 C shows platelet counts pertaining to 1000 k/μl, 500 k/μl, and 250 k/μl, respectively.

FIG. 27 shows brightfield, fluorescence, and overlaid microscope images of platelets loaded with fluorescently labeled Paclitaxel.

FIG. 28 shows percent transfection of fresh platelets with fluorescent siRNA incubated for 2 hours in different liquid transfection media. HMTA=HEPES-buffered Tryode's Solution. Opti-MEM=Opti-MEM™ (ThermoSci). EtOH=Ethanol.

FIG. 29 shows geometric mean fluorescence intensity of fresh platelets incubated in different liquid transfection media containing fluorescent siRNA. HMTA=HEPES-buffered Tryode's Solution. Opti-MEM=Opti-MEM™ (ThermoSci). EtOH=Ethanol.

FIG. 30 shows percent transfection of stored platelets incubated in liquid transfection media containing fluorescent siRNA at different time points indicated on the X axis.

FIG. 31 shows flow cytometry data of percent transfection of stored platelets when incubated under indicated conditions. Oligo=BLOCK-iT™ Fluorescent oligo (ThermoSci). Opti=Opti-MEM™ (ThermoSci). Lipo=Lipofectamine™ RNAiMAX (ThermoSci).

FIG. 32 shows percent transfection of stored platelets when incubated under indicated conditions. Oligo=BLOCK-iT™ Fluorescent oligo (ThermoSci). Opti=Opti-MEM™ (ThermoSci). Lipo=Lipofectamine™ RNAiMAX (ThermoSci).

FIG. 33 shows maximum percent aggregation, when incubated for 5 hours, of siRNA-loaded stored platelets after exposure to platelet aggregation agonists Thrombin Receptor Agonist Peptide (TRAP), collagen, or arachidonic acid. hIDSP=originating stored platelets.

FIG. 34A and FIG. 34B provide raw aggregometry data for siRNA-loaded stored platelets incubated with platelet aggregation agonists. Channel 1=Negative Control. Channels 2-4=TRAP incubated. Channels 5-6=collagen incubated. Channels 7-8—arachidonic acid incubated.

FIG. 35 shows flow cytometry data for siRNA-loaded fresh platelets after 5 minute exposure to platelet aggregation agonists adenosine diphosphate (ADP) and TRAP as compared to an unexposed control.

FIG. 36 shows flow cytometry data for siRNA retention in rehydrated Thrombosomes. Sham Post-Lyo=Negative Control Thrombosomes. FITC-Oligo Post-lyo=BLOCK-iT™ Fluorescent Oligo transfected post-lyophilization. FITC-Oligo Pre-lyo=BLOCK-iT™ Fluorescent Oligo transfected pre-lyophilization.

FIG. 37 shows fluorescence microscopy data for siRNA retention in rehydrated Thrombosomes transfected with fluorescent siRNA. Image is shown at 100× magnification. Scale bar is 10 μm

FIG. 38 provides raw occlusion time course data for Thrombosomes transfected with or without fluorescent siRNA.

FIG. 39 shows occlusion rates for Thrombosomes transfected with or without fluorescent siRNA.

FIG. 40 shows the percentage of platelets in Test Groups A and B that are transfected under the conditions described in Example 17.

FIG. 41 shows the fluorescence intensity of platelets in Test Groups A and B that are transfected under the conditions described in Example 17.

FIG. 42 shows a normalized histogram of fluorescence intensity in FITC for all single platelet events collected for Test Groups A and B that are transfected under the conditions described in Example 17.

FIG. 43 shows flow cytometry plots for platelets transfected with fluorescently labeled miR-34a under the conditions described in Example 18.

FIG. 44 shows fluorescence plots for non-transfected platelets, transfected platelets, and transfected cryopreserved platelets.

FIG. 45 shows plots of flow pressure vs. time in a channel occlusion test for non-transfected platelets and transfected cryopreserved platelets.

FIG. 46 shows saponin-mediated permeabilization of platelets measured using standard light transmission aggregometry (LTA).

FIG. 47 provides median fluorescence measured using flow cytometry of platelets incubated in the presence of saponin to show the effect of saponin on the permeabilization of platelet cell membranes.

FIG. 48 shows flow cytometry data providing endocytotic loading efficiency of BODIPY-vancomycin, FITC-bovine IgG, Fab2, and FITC-albumin in platelets.

FIG. 49 shows flow cytometry data providing endocytotic loading efficiency of fluorescently-labeled BODIPY-vancomycin was loaded into platelets via fluid phase endocytosis over different incubation time periods.

FIG. 50 shows fluorescent intensity for samples prepared with different hypertonic pre-treatment solutions containing variable amounts of dextrose.

FIG. 51 shows cell counts (gated by size) for samples prepared with differing hypertonic pre-treatment solutions containing variable amounts of dextrose.

FIG. 52 shows cell size for samples prepared with differing hypertonic pre-treatment solutions containing variable amounts of dextrose.

FIG. 53 shows platelet aggregation in response to collagen for samples prepared with differing hypertonic pre-treatment solutions containing variable amounts of dextrose.

FIG. 54 is a histogram of platelet+/−Phalloidin-CF488A FITC-H intensity on a bio-exponential scale.

FIG. 55 shows mean platelet FITC-H intensity by flow cytometry for samples with or without 5 U/mL Phalloidin-CF488A and with or without electroporation.

FIG. 56 is a histogram of platelet+/−Streptavidin-Dylight 488 FITC-H intensity on a bio-exponential scale.

FIG. 57 shows mean platelet FITC-H intensity by flow cytometry for samples with or without 150 μg/mL Streptavidin-Dylight 488 and with or without electroporation.

FIG. 58 is a histogram of Lucifer yellow fluorescence in platelets under various loading conditions.

FIG. 59 shows Lucifer yellow fluorescence in platelets under various loading conditions.

FIG. 60A and FIG. 60B show detection of Cy5-linked EGFP mRNA after 30 minutes of incubation in samples A, B, C, G (FIG. 60A) and samples D, E, F, and G (FIG. 60B).

FIG. 61A and FIG. 61B show detection of Cy5-linked EGFP mRNA after 120 minutes of incubation in samples A, B, C, G (FIG. 61A) and samples D, E, F and G (FIG. 61B).

FIG. 62 is a graph showing mRNA loaded platelet concentration (plts/μl) measured at 30 minutes and 120 minutes over an incubation time of 120 minutes.

FIG. 63 is a graph showing mRNA loaded platelet forward scatter height (FSC-H) measured by flow cytometry at 30 minutes and 120 minutes over an incubation time of 120 minutes.

FIG. 64 is a graph showing mRNA loaded platelet Cy5 mean fluorescent intensity measured at 30 minutes and 120 minutes over an incubation time of 120 minutes.

FIG. 65 is a graph showing Cy5-H positive mRNA loaded platelets percentage measured at 30 minutes and 120 minutes over an incubation time of 120 minutes.

FIG. 66 is a flow cytometry histogram showing measurement of mRNA loaded platelet Cy5-H after 30 minutes of incubation as measured by count [10³] in samples A, E, G, I, and J.

FIG. 67 is a flow cytometry histogram showing measurement of mRNA loaded platelet Cy5-H after 120 minutes of incubation as measured by count [10³] in samples A, E, G, I, and J.

FIG. 68 is a graph showing mRNA loaded platelet concentration (plts/μl) measured at 30 minutes and 120 minutes over an incubation time of 120 minutes in samples A, E, G, I, and J.

FIG. 69 is a graph showing mRNA loaded platelet forward scatter height (FSC-H) measured by flow cytometry at 30 minutes and 120 minutes over an incubation time of 120 minutes in samples A, E, G, I, and J.

FIG. 70 is a graph showing mRNA loaded platelet Cy5-H measured at 30 minutes and 120 minutes over an incubation time of 120 minutes in samples A, E, G, I, and J.

FIG. 71 is a graph showing mRNA loaded platelet Cy5 positivity percentage measured at 30 minutes and 120 minutes over an incubation time of 120 minutes in samples A, E, G, I, and J.

FIG. 72 is a graph showing a flow cytometry histogram for sample E measured at 30 minutes and 120 minutes over an incubation time of 120 minutes.

FIG. 73 is a graph showing a flow cytometry histogram for sample G measured at 30 minutes and 120 minutes over an incubation time of 120 minutes.

FIG. 74 is a graph showing a flow cytometry histogram for sample I measured at 30 minutes and 120 minutes over an incubation time of 120 minutes.

As used herein and in the appended claims, the singular forms “a”, “an”, and “the” include plural referents unless the context clearly dictates otherwise. Thus, for example, reference to “a platelet” includes a plurality of such platelets. Furthermore, the use of terms that can be described using equivalent terms include the use of those equivalent terms. Thus, for example, the use of the term “subject” is to be understood to include the terms “patient”, “individual” and other terms used in the art to indicate one who is subject to a treatment.

Unless defined otherwise, all technical and scientific terms used herein have the same meaning as commonly understood by one of ordinary skill in the art to which the term belongs. Although any methods and materials similar or equivalent to those described herein can be used in the practice or testing of the present disclosure, the preferred methods and materials are now described. All publications mentioned herein are incorporated herein by reference to disclose and describe the methods and/or materials in connection with which the publications are cited. The present disclosure is controlling to the extent it conflicts with any incorporated publication. Any Headings herein are for convenience only and are not intended to be limiting, and it will be understood that the disclosure including aspects and embodiments provided within one section herein can be combined with the disclosure includes aspects and embodiments provided within any other section herein.

### DETAILED DESCRIPTION

Unless defined otherwise, all technical and scientific terms used herein have the same meaning as commonly understood by one of ordinary skill in the art to which the term belongs. Although any methods and materials similar or equivalent to those described herein can be used in the practice or testing of the present disclosure, the preferred methods and materials are now described. All publications mentioned herein are incorporated herein by reference to disclose and describe the methods and/or materials in connection with which the publications are cited. The present disclosure is controlling to the extent it conflicts with any incorporated publication.

It is to be understood that the terminology used herein is for the purpose of describing particular aspects and embodiments only, and is not intended to be limiting. Further, where a range of values is disclosed, the skilled artisan will understand that all other specific values within the disclosed range are inherently disclosed by these values and the ranges they represent without the need to disclose each specific value or range herein. For example, a disclosed range of 1-10 includes 1-9, 1-5, 2-10, 3.1-6, 1, 2, 3, 4, 5, and so forth. In addition, each disclosed range includes up to 5% lower for the lower value of the range and up to 5% higher for the higher value of the range. For example, a disclosed range of 4-10 includes 3.8-10.5. This concept is captured in this document by the term “about”.

As used herein and in the appended claims, the term “platelet” can include whole platelets, fragmented platelets, platelet derivatives, or thrombosomes. Thus, for example, reference to “drug-loaded platelets” may be inclusive of drug-loaded platelets as well as drug-loaded platelet derivatives or drug-loaded thrombosomes, unless the context clearly dictates a particular form.

As used herein and in the appended claims, the singular forms “a”, “an”, and “the” include plural referents unless the context clearly dictates otherwise. Thus, for example, reference to “a platelet” includes a plurality of such platelets. Furthermore, the use of terms that can be described using equivalent terms include the use of those equivalent terms. Thus, for example, the use of the term “subject” is to be understood to include the terms “patient”, “individual” and other terms used in the art to indicate one who is subject to a treatment.

In some embodiments, rehydrating the drug-loaded platelets comprises adding to the platelets an aqueous liquid. In some embodiments, the aqueous liquid is water. In some embodiments, the aqueous liquid is an aqueous solution. In some embodiments, the aqueous liquid is a saline solution. In some embodiments, the aqueous liquid is a suspension.

In some embodiments, the rehydrated platelets have coagulation factor levels showing all individual factors (e.g., Factors VII, VIII and IX) associated with blood clotting at 40 international units (IU) or greater.

In some embodiments, the dried platelets, such as freeze-dried platelets, have less than about 10%, such as less than about 8%, such as less than about 6%, such as less than about 4%, such as less than about 2%, such as less than about 0.5% crosslinking of platelet membranes via proteins and/or lipids present on the membranes. In some embodiments, the rehydrated platelets, have less than about 10%, such as less than about 8%, such as less than about 6%, such as less than about 4%, such as less than about 2%, such as less than about 0.5% crosslinking of platelet membranes via proteins and/or lipids present on the membranes.

In some embodiments, the drug-loaded platelets and the dried platelets, such as freeze-dried platelets, having a particle size (e.g., diameter, max dimension) of at least about 0.2 μm (e.g., at least about 0.3 μm, at least about 0.4 μm, at least about 0.5 μm, at least about 0.6 μm, at least about 0.7 μm, at least about 0.8 μm, at least about 0.9 μm, at least about 1.0 μm, at least about 1.0 μm, at least about 1.5 μm, at least about 2.0 μm, at least about 2.5 μm, or at least about 5.0 μm). In some embodiments, the particle size is less than about 5.0 μm (e.g., less than about 2.5 μm, less than about 2.0 μm, less than about 1.5 μm, less than about 1.0 μm, less than about 0.9 μm, less than about 0.8 μm, less than about 0.7 μm, less than about 0.6 μm, less than about 0.5 μm, less than about 0.4 μm, or less than about 0.3 μm). In some embodiments, the particle size is from about 0.3 μm to about 5.0 μm (e.g., from about 0.4 μm to about 4.0 μm, from about 0.5 μm to about 2.5 μm, from about 0.6 μm to about 2.0 μm, from about 0.7 μm to about 1.0 μm, from about 0.5 μm to about 0.9 μm, or from about 0.6 μm to about 0.8 μm).

In some embodiments, at least 50% (e.g., at least about 55%, at least about 60%, at least about 65%, at least about 70%, at least about 75%, at least about 80%, at least about 85%, at least about 90%, at least about 95%, or at least about 99%) of platelets and/or the dried platelets, such as freeze-dried platelets, have a particle size in the range of about 0.3 μm to about 5.0 μm (e.g., from about 0.4 μm to about 4.0 μm, from about 0.5 μm to about 2.5 μm, from about 0.6 μm to about 2.0 μm, from about 0.7 μm to about 1.0 μm, from about 0.5 μm to about 0.9 μm, or from about 0.6 μm to about 0.8 μm). In some embodiments, at most 99% (e.g., at most about 95%, at most about 80%, at most about 75%, at most about 70%, at most about 65%, at most about 60%, at most about 55%, or at most about 50%) of platelets and/or the dried platelets, such as freeze-dried platelets, are in the range of about 0.3 μm to about 5.0 μm (e.g., from about 0.4 μm to about 4.0 μm, from about 0.5 μm to about 2.5 μm, from about 0.6 μm to about 2.0 μm, from about 0.7 μm to about 1.0 μm, from about 0.5 μm to about 0.9 μm, or from about 0.6 μm to about 0.8 μm). In some embodiments, about 50% to about 99% (e.g., about 55% to about 95%, about 60% to about 90%, about 65% to about 85, about 70% to about 80%) of platelets and/or the dried platelets, such as freeze-dried platelets, are in the range of about 0.3 μm to about 5.0 μm (e.g., from about 0.4 μm to about 4.0 μm, from about 0.5 μm to about 2.5 μm, from about 0.6 μm to about 2.0 μm, from about 0.7 μm to about 1.0 μm, from about 0.5 μm to about 0.9 μm, or from about 0.6 μm to about 0.8 μm).

In some embodiments, platelets are isolated prior to treating the platelets with a drug.

Accordingly, in some embodiments, the method for preparing drug-loaded platelets comprises:
a) isolating platelets, for example in a liquid medium;
   and
b) treating the platelets with a drug and with a loading buffer comprising a salt, abase, a loading agent, and optionally ethanol, to form the drug-loaded platelets.

Accordingly, in some embodiments, the method for preparing drug-loaded platelets comprises:
a) isolating platelets, for example in a liquid medium;
b) treating the platelets with a drug to form a first composition; and
c) treating the first composition with a buffer comprising a salt, a base, a loading agent, and optionally at least one organic solvent to form the drug-loaded platelets.

In some embodiments, suitable organic solvents include, but are not limited to alcohols, esters, ketones, ethers, halogenated solvents, hydrocarbons, nitriles, glycols, alkyl nitrates, water or mixtures thereof. In some embodiments, suitable organic solvents includes, but are not limited to methanol, ethanol, n-propanol, isopropanol, acetic acid, acetone, methyl ethyl ketone, methyl isobutyl ketone, methyl acetate, ethyl acetate, isopropyl acetate, tetrahydrofuran, isopropyl ether (IPE), tert-butyl methyl ether, dioxane (e.g., 1,4-dioxane), acetonitrile, propionitrile, methylene chloride, chloroform, toluene, anisole, cyclohexane, hexane, heptane, ethylene glycol, nitromethane, dimethylformamide, dimethyl sulfoxide, N-methyl pyrrolidone, dimethylacetamide, and combinations thereof.

Accordingly, in some embodiments, the method for preparing drug-loaded platelets comprises:
a) isolating platelets, for example in a liquid medium;
b) treating the platelets with a buffer comprising a salt, a base, a loading agent, and optionally at least one organic solvent, to form a first composition; and
c) treating the first composition with a drug, to form the drug-loaded platelets.

In some embodiments, no solvent is used. Thus, in some embodiments, the method for preparing drug-loaded platelets comprises:
a) isolating platelets, for example in a liquid medium;
   and
b) treating the platelets with a drug and with a loading buffer comprising a salt, abase, and a loading agent, to form the drug-loaded platelets,
   wherein the method does not comprise treating the platelets with an organic solvent such as ethanol.

Thus, in some embodiments, the method for preparing drug-loaded platelets comprises:
a) isolating platelets, for example in a liquid medium;
b) treating the platelets with a drug to form a first composition; and
c) treating the first composition with a buffer comprising a salt, a base, and a loading agent, to form the drug-loaded platelets,
   wherein the method does not comprise treating the platelets with an organic solvent such as ethanol and the method does not comprise treating the first composition with an organic solvent such as ethanol.

Thus, in some embodiments, the method for preparing drug-loaded platelets comprises:
a) isolating platelets, for example in a liquid medium;
b) treating the platelets with a buffer comprising a salt, a base, and a loading agent, to form a first composition; and
c) treating the first composition with a drug, to form the drug-loaded platelets.
   wherein the method does not comprise treating the platelets with an organic solvent such as ethanol and the method does not comprise treating the first composition with an organic solvent such as ethanol.

In some embodiments, isolating platelets comprises isolating platelets from blood.

In some embodiments, platelets are donor-derived platelets. In some embodiments, platelets are obtained by a process that comprises an apheresis step.

In some embodiments, platelets are derived in vitro. In some embodiments, platelets are derived or prepared in a culture prior to treating the platelets with a drug. In some embodiments, preparing the platelets comprises deriving or growing the platelets from a culture of megakaryocytes. In some embodiments, preparing the platelets comprises deriving or growing the platelets (or megakaryocytes) from a culture of human pluripotent stem cells (PCSs), including embryonic stem cells (ESCs) and/or induced pluripotent stem cells (iPSCs).

Accordingly, in some embodiments, the method for preparing drug-loaded platelets comprises:
a) preparing platelets;
   and
b) treating the platelets with a drug and with a loading buffer comprising a salt, a base, a loading agent, and optionally at least one organic solvent, to form the drug-loaded platelets.

Accordingly, in some embodiments, the method for preparing drug-loaded platelets comprises:
a) preparing platelets;
b) treating the platelets with a drug to form a first composition; and
c) treating the first composition with a buffer comprising a salt, a base, a loading agent, and optionally at least one organic solvent, to form the drug-loaded platelets.

Accordingly, in some embodiments, the method for preparing drug-loaded platelets comprises:
a) preparing platelets;
b) treating the platelets with a buffer comprising a salt, a base, a loading agent, and optionally at least one organic solvent, to form a first composition; and
c) treating the first composition with a drug, to form the drug-loaded platelets.

In some embodiments, no solvent is used. Thus, in some embodiments, the method for preparing drug-loaded platelets comprises:
a) preparing platelets;
   and
b) treating the platelets with a drug and with a loading buffer comprising a salt, a base, and a loading agent, to form the drug-loaded platelets,
   wherein the method does not comprise treating the platelets with an organic solvent such as ethanol.

Thus, in some embodiments, the method for preparing drug-loaded platelets comprises:
a) preparing platelets;
b) treating the platelets with a drug to form a first composition; and
c) treating the first composition with a buffer comprising a salt, a base, and a loading agent, to form the drug-loaded platelets,
   wherein the method does not comprise treating the platelets with an organic solvent such as ethanol and the method does not comprise treating the first composition with an organic solvent such as ethanol.

Thus, in some embodiments, the method for preparing drug-loaded platelets comprises:
a) preparing platelets;
b) treating the platelets with a buffer comprising a salt, a base, and a loading agent, to form a first composition; and
c) treating the first composition with a drug, to form the drug-loaded platelets.
   wherein the method does not comprise treating the platelets with an organic solvent such as ethanol and the method does not comprise treating the first composition with an organic solvent such as ethanol.

In some embodiments, the loading agent is a saccharide. In some embodiments, the saccharide is a monosaccharide. In some embodiments, the saccharide is a disaccharide. In some embodiments, the saccharide is a non-reducing disaccharide. In some embodiments, the saccharide is sucrose, maltose, trehalose, glucose (e.g., dextrose), mannose, or xylose. In some embodiments, the loading agent is a starch.

In some embodiments, the loading agent is a carrier protein. In some embodiments, the carrier protein is albumin. In some embodiments, the carrier protein is bovine serum albumin (BSA).

As used herein, the term “drug” refers to any agent suitable for the treatment of cancer other than a messenger RNA (mRNA), a microRNA (also known as miRNA) and/or a small interfering RNA (also known as siRNA, short interfering RNA, or silencing RNA).

As used herein, the term “mRNA” refers to a single-stranded ribonucleic acid molecule used by cells for the translation of DNA into protein. Many mRNAs are naturally-occurring, but mRNAs can also be synthesized by those of ordinary skill in the art. Mature mRNAs can vary greatly in size and composition. mRNAs are necessary components of protein synthesis after exportation from the nucleus to the cytoplasm of the cell.

As used herein, the term “microRNA” refers to a ribonucleic acid duplex that targets and silences an mRNA molecule. Many miRNAs are naturally-occurring, but miRNAs can also be synthesized by those of ordinary skill in the art. Mature miRNAs are generally 19-25 nucleotides in length, have 3′ overhangs of two nucleotides, target multiple mRNAs and are typically only partially complementary to their target mRNAs. miRNAs typically function by repressing translation and facilitating mRNA degradation.

As used herein, the term “small interfering RNA” refers to a double-stranded RNA that targets and silences an mRNA molecule. Many siRNAs are naturally-occurring, but siRNAs can also be synthesized by those of ordinary skill in the art. siRNA are generally derived from strands of exogenous growing (originating from outside an organism) RNA, which is taken up by the cell and undergoes further processing. Mature siRNAs are generally 19-27 nucleotides in length, have 3′ overhangs of two nucleotides at each end that can be used to “interfere” with the translation of proteins by binding to and promoting the degradation of messenger RNA at specific sequences. Each siRNA strand has a 5′ phosphate group and a 3′ hydroxyl (OH) group. siRNA can be produced from dsRNA or hairpin looped RNA and processed into siRNAs by the Dicer enzyme. siRNA can also be incorporated into a multi-subunit protein complex called RNA-induced silencing complex (RISC).

miRNAs and siRNAs are distinct from other types of RNA molecules including, without limitation, messenger RNA (“mRNA), ribosomal RNA (“rRNA”), small nuclear RNA (“snRNA”), transfer RNA (“tRNA”), and short hairpin RNA (“shRNA”). rRNA, snRNA, tRNA, and shRNA are all encompassed within the term “drug” as used herein. mRNA, rRNA, snRNA, and tRNA are canonical classes of RNA molecules, the function and structure of which are well-known to those of ordinary skill in the art.

shRNAs are short linear RNA molecules, portions of which associate with each other via base pairing to form a double stranded stem region (as opposed to the fully double stranded siRNAs), resulting in a characteristic “hairpin” or loop at one end of the molecule. Unlike miRNAs and siRNAs, shRNAs are typically introduced into cells using methods that differ from the methods used for introducing miRNA and siRNA (e.g., using transfection methods). For example, shRNAs can be introduced via plasmids or, alternatively, through viral or bacterial vectors. Both of these methods are DNA-based techniques, where the shRNA is transcribed, processed by the enzyme Drosha, and then further processed into siRNAs, by the Dicer enzyme, to eventually mediate RNAi.

As used herein, “thrombosomes” (sometimes also herein called “Tsomes” or “Ts”, particularly in the Examples and Figures) are platelet derivatives that have been treated with an incubating agent (e.g., any of the incubating agents described herein) and lyopreserved (e.g., freeze-dried). In some cases, thrombosomes can be prepared from pooled platelets. Thrombosomes can have a shelf life of 2-3 years in dry form at ambient temperature and can be rehydrated with sterile water within minutes for immediate infusion.

In some embodiments, the drug is selected from the group consisting of one of the following:
i. a small molecule (that is, an organic compound having a molecular weight up to about 2 KDalton);
ii. a protein;
iii. an oligopeptide;
iv. a non-miRNA nucleic acid, a non-siRNA, and/or a non-mRNA (e.g., non-miRNA, DNA, other naturally or non-naturally occurring nucleic acids, including various modifications thereof);
   and
v. an aptamer.

In various methods described herein, platelets are loaded with one or more any of a variety of drugs. In some embodiments, platelets are loaded with a small molecule. For example, platelets can be loaded with one or more of vemurafenib (ZELBORAF®), dabrafenib (TAFINLAR®), encorafenib (BRAFTOVI™), BMS-908662 (XL281), sorafenib, LGX818, PLX3603, RAF265, RO5185426, GSK2118436, ARQ 736, GDC-0879, PLX-4720, AZ304, PLX-8394, HM95573, RO5126766, LXH254, trametinib (MEKINIST®, GSK1120212), cobimetinib (COTELLIC®), binimetinib (MEKTOVI®, MEK162), selumetinib (AZD6244), PD0325901, MSC1936369B, SHR7390, TAK-733, CS3006, WX-554, PD98059, C11040 (PD184352), hypothemycin, FRI-20 (ON-01060), VTX-11e, 25-OH-D3-3-BE (B3CD, bromoacetoxycalcidiol), FR-180204, AEZ-131 (AEZS-131), AEZS-136, AZ-13767370, BL-EI-001, LY-3214996, LTT-462, KO-947, KO-947, MK-8353 (SCH900353), SCH772984, ulixertinib (BVD-523), CC-90003, GDC-0994 (RG-7482), ASN007, FR148083, 5-7-oxozeaenol, 5-iodotubercidin, GDC0994, ONC201, buparlisib (BKM120), alpelisib (BYL719), WX-037, copanlisib (ALIQOPA™, BAY80-6946), dactolisib (NVP-BEZ235, BEZ-235), taselisib (GDC-0032, RG7604), sonolisib (PX-866), CUDC-907, PQR309, ZSTK474, SF1126, AZD8835, GDC-0077, ASN003, pictilisib (GDC-0941), pilaralisib (XL147, SAR245408), gedatolisib (PF-05212384, PKI-587), serabelisib (TAK-117, MLN1117, INK 1117), BGT-226 (NVP-BGT226), PF-04691502, apitolisib (GDC-0980), omipalisib (GSK2126458, GSK458), voxtalisib (XL756, SAR245409), AMG 511, CH5132799, GSK1059615, GDC-0084 (RG7666), VS-5584 (SB2343), PKI-402, wortmannin, LY294002, PI-103, rigosertib, XL-765, LY2023414, SAR260301, KIN-193 (AZD-6428), GS-9820, AMG319, GSK2636771, NL-71-101, H-89, GSK690693, CCT128930, AZD5363, ipatasertib (GDC-0068, RG7440), A-674563, A-443654, AT7867, AT13148, uprosertib, afuresertib, DC120, 2-[4-(2-aminoprop-2-yl)phenyl]-3-phenylquinoxaline, MK-2206, edelfosine, erucylphophocholine, erufosine, SR13668, OSU-A9, PH-316, PHT-427, PIT-1, DM-PIT-1, triciribine (triciribine phosphate monohydrate), API-1, N-(4-(5-(3-acetamidophenyl)-2-(2-aminopyridin-3-yl)-3H-imidazo[4,5-b] pyridin-3-yl)benzyl)-3-fluorobenzamide, ARQ092, BAY 1125976, 3-oxo-tirucallic acid, lactoquinomycin, boc-Phe-vinyl ketone, Perifosine (D-21266), TCN, TCN-P, GSK2141795, MLN0128, AZD-2014, CC-223, AZD2014, CC-115, everolimus (RAD001), temsirolimus (CCI-779), ridaforolimus (AP-23573), tipifarnib, BMS-214662, L778123, L744832, FTI-277, PRI-724, CWP232291, PNU74654, PKF115-584, PKF118-744, PKF118-310, PFK222-815, CGP 049090, ZTM000990, BC21, methyl 3-{[(4-methylphenyl)sulfonyl]amino}benzoate (MSAB), AV65, iCRT3, iCRT5, iCRT14, SM04554, LGK 974, XAV939, curcumin (e.g., Meriva®), DIF-1, genistein, NSC668036, FJ9, BML-286 (3289-8625), IWP, IWP-1, IWP-2, JW55, G007-LK, pyrvinium, foxy-5, Wnt-5a, ipafricept (OMP-54F28), vantictumab (OMP-18R5), SM04690, SM04755, nutlin-3a, IWR1, JW74, okadaic acid, SB239063, SB203580, adenosine diphosphate (hydroxymethyl)pyrrolidinediol (ADP-HPD), 2-[4-(4-fluorophenyl)piperazin-1-yl]-6-methylpyrimidin-4(3H)-one, PJ34, J01-017a, IC261, PF670462, bosutinib (BOSULIF®), PHA665752, imatinib (GLEEVEC®), ICG-001, Rp-8-Br-cAMP, SDX-308, WNT974, CGX1321, ETC-1922159, AD-REIC/Dkk3, WIKI4, windorphen, NTRC 0066-0, CFI-402257, a (5,6-dihydro)pyrimido[4,5-e]indolizine, BOS172722, S63845, AZD5991, AMG 176, 483-LM, MIK665, TASIN-1 (Truncated APC Selective Inhibitor), osimertinib (AZD9291, merelectinib, TAGRISSO™), erlotinib (TARCEVA®), gefitinib (IRESSA®), neratinib (HKI-272, NERLYNX®), lapatinib (TYKERB®), vetanib (CAPRELSA®), rociletinib (CO-1686), olmutinib (OLITA™, HM61713, BI-1482694), naquotinib (ASP8273), nazartinib (EGF816, NVS-816), PF-06747775, icotinib (BPI-2009H), afatinib (BIBW 2992, GILOTRIF®), dacomitinib (PF-00299804, PF-804, PF-299, PF-299804), avitinib (AC0010), AC0010MA EAI045, canertinib (CI-1033), poziotinib (NOV120101, HM781-36B), AV-412, WZ4002, brigatinib (AP26113, ALUNBRIG®), pelitinib (EKB-569), tarloxotinib (TH-4000, PR610), BPI-15086, Hemay022, ZN-e4, tesevatinib (KD019, XL647), YH25448, epitinib (HMPL-813), CK-101, MM-151, AZD3759, ZD6474, PF-06459988, varlintinib (ASLAN001, ARRY-334543), AP32788, HLX07, D-0316, AEE788, HS-10296, GW572016, pyrotinib (SHR1258), palbociclib, ribociclib, abemaciclib, olaparib, veliparib, iniparib, rucaparib, CEP-9722, E7016, E7449, PRN1371, BLU9931, FIIN-4, H3B-6527, NVP-BGJ398, ARQ087, TAS-120, CH5183284, Debio 1347, INCB054828, JNJ-42756493 (erdafitinib), rogaratinib (BAY1163877), FIIN-2, LY2874455, lenvatinib (E7080), ponatinib (AP24534), regorafenib (BAY 73-4506), dovitinib (TKI258), lucitanib (E3810), cediranib (AZD2171), nintedanib (OFEV®, BIBF 1120), brivanib (BMS-540215), ASP5878, AZD4547, BGJ398 (infigratinib), E7090, HMPL-453, MAX-40279, XL999, orantinib (SU6668), pazopanib (VOTRIENT®), anlotinib, AL3818, PRIMA-1 (p53 reactivation induction of massive apoptosis-1), APR-246 (PRIMA-1MET), 2-sulfonylpyrimidines such as PK11007, pyrazoles such as PK7088, zinc metallochaperone-1 (ZMC1; NSC319726/ZMC 1), a thiosemicarbazone (e.g., COTI-2), CP-31398, STIMA-1 (SH Group-Targeting Compound That Induces Massive Apoptosis), MIRA-1 (NSC19630) and its analogs, e.g., MIRA-2 and MIRA-3, RITA (NSC652287), chetomin (CTM), stictic acid (NSC87511), p53R3, SCH529074, WR-1065, arsenic compounds, gambogic acid, spautin-1, YK-3-237, NSC59984, disulfiram (DSF), G418, RETRA (reactivate transcriptional activity), PD0166285, 17-AAG, geldanamycin, ganetespib, AUY922, IPI-504, vorinostat/SAHA, romidepsin/depsipeptide, HBI-8000, RG7112 (R05045337), R05503781, MI-773 (SAR405838), DS-3032b, AM-8553, AMG 232, MI-219, MI-713, MI-888, TDP521252, NSC279287, PXN822, ATSP-7041, spiroligomer, PK083, PK5174, PK5196, nutlin 3a, RG7388, Ro-2443, FTY-720, ceramide, OP449, vatalanib (PTK787/ZK222584), TKI-538, sunitinib (SU11248, SUTENT®), thalidomide, lenalidomide (REVLIMID®), axitinib (AG013736, INLYTA®), RXC0004, ETC-159, LGK974, WNT-C59, AZD8931, AST1306, CP724714, CUDC101, TAK285, AC480, DXL-702, E-75, PX-104.1, ZW25, CP-724714, irbinitinib (ARRY-380, ONT-380), TAS0728, AST-1306, AEE-788, perlitinib (EKB-569), PKI-166, D-69491, HKI-357, AC-480 (BMS-599626), RB-200h, ARRY-334543 (ARRY-543, ASLAN001), CUDC-101, IDM-1, decitabine, cytosine arabinoside, ORY1001 (RG6016), GSK2879552, INCB059872, IMG7289, CC90011, MI1, MI2, MI3, Mi2-2 (MI-2-2), MI463, MI503, MIV-6R, EPZ004777, EPZ-5676, SGC0946, CN-SAH, SYC-522, SAH, SYC-534, MM-101, MM-102, MM-103, MM-401, WDR5-0101, WDR5-0102, WDR5-0103, OICR-9429, tivantinib (ARQ 197), golvatinib (E7050), cabozantinib (XL 184, BMS-907351), foretinib (GSK1363089), crizotinib (PF-02341066), MK-2461, BPI-9016M, TQ-B3139, MGCD265, MK-8033, capmatinib (INC280, INCB28060), tepotinib (MSC2156119J, EMD1214063), CE-35562, AMG-337, AMG-458, PHA-665725, PF-04217903, SU11274, PHA-665752, HS-10241, ARGX-111, glumetinib (SCC244), EMD 1204831, AZD6094 (savolitinib, volitinib, HMPL-504), PLB1001, ABT-700, AMG 208, INCB028060, AL2846, HTI-1066, PT2385, PT2977, 17 allylamino-17-demethoxygeldanamycin, eribulin (HALAVEN®, E389, ER-086526), ibrutinib (PCI-32675, Imbruvica®) (1-[(3R)-3-[4-amino-3-(4-phenoxyphenyl)pyrazolo[3,4-d]pyrimidin-1-yl]piperidin-1-yl]prop-2-en-1-one); AC0058 (AC0058TA); N-(3-((2-((3-fluoro-4-(4-methylpiperazin-1-yl)phenyl)amino)-7H-pyrrolo[2,3-d]pyrimidin-4-yl)oxy)phenyl)acrylamide; acalabrutinib (ACP-196, Calquence®, rINN) (4-[8-amino-3-[(2S)-1-but-2-ynoylpyrrolidin-2-yl]imidazo[1,5-a]pyrazin-1-yl]-N-pyridin-2-ylbenzamide); zanubrutinib (BGB-3111) ((7R)-2-(4-phenoxyphenyl)-7-(1-prop-2-enoylpiperidin-4-yl)-1,5,6,7-tetrahydropyrazolo[1,5-a]pyrimidine-3-carboxamide); spebrutinib (AVL-292, 1202757-89-8, Cc-292) (N-[3-[[5-fluoro-2-[4-(2-methoxyethoxy)anilino]pyrimidin-4-yl]amino]phenyl]prop-2-enamide); poseltinib (HM71224, LY3337641) (N-[3-[2-[4-(4-methylpiperazin-1-yl)anilino]furo[3,2-d]pyrimidin-4-yl]oxyphenyl]prop-2-enamide); evobrutinib (MSC 2364447, M-2951) (1-[4-[[[6-amino-5-(4-phenoxyphenyl)pyrimidin-4-yl]amino]methyl]piperidin-1-yl]prop-2-en-1-one); tirabrutinib (ONO-4059, GS-4059, ONO/GS-4059, ONO-WG-307) (1-[4-[[[6-amino-5-(4-phenoxyphenyl)pyrimidin-4-yl]amino]methyl]piperidin-1-yl]prop-2-en-1-one); vecabrutinib (SNS-062) ((3R,4S)-1-(6-amino-5-fluoropyrimidin-4-yl)-3-[(3R)-3-[3-chloro-5-(trifluoromethyl)anilino]-2-oxopiperidin-1-yl]piperidine-4-carboxamide); dasatinib (Sprycel®; BMS-354825) (N-(2-chloro-6-methylphenyl)-2-[[6-[4-(2-hydroxyethyl)piperazin-1-yl]-2-methylpyrimidin-4-yl]amino]-1,3-thiazole-5-carboxamide); PRN1008, PRN473, ABBV-105, CG′806, ARQ 531, BIIB068, AS871, CB1763, CB988, GDC-0853, RN486, GNE-504, GNE-309, BTK Max, CT-1530, CGI-1746, CGI-560, LFM A13, TP-0158, dtrmwxhs-12, CNX-774, entrectinib, nilotinib, 1-((3S,4R)-4-(3-fluorophenyl)-1-(2-methoxyethyl)pyrrolidin-3-yl)-3-(4-methyl-3-(2-methylpyrimidin-5-yl)-1-phenyl-1H-pyrazol-5-yl)urea, AG 879, AR-772, AR-786, AR-256, AR-618, AZ-23, AZ623, DS-6051, Go 6976, GNF-5837, GTx-186, GW 441756, LOXO-101, LOXO-195, MGCD516, PLX7486, RXDX101, TPX-0005, TSR-011, venetoclax (ABT-199, RG7601, GDC-0199), navitoclax (ABT-263), ABT-737, TW-37, sabutoclax, obatoclax, BIX-01294 (BIX), UNC0638, A-366, UNC0642, DCG066, UNC0321, BRD 4770, UNC 0224, UNC 0646, UNC0631, BIX-01338, INNO-406, KX2-391, saracatinib, PP1, PP2, ruxolitinib, lestaurtinib (CEP-701), momelotinib (GS-0387, CYT-387), pacritinib (SB1518), fedratinib (SAR302503), BI2536, BI6727, GSK461364, amsacrine, azacitidine, busulfan, carboplatin, capecitabine, chlorambucil, cisplatin, cyclophosphamide, cytarabine, dacarbazine, daunorubicin, docetaxel, doxifluridine, doxorubicin, epirubicin, etoposide, fiudarabine, floxuridine, fludarabine, fluorouracil, gemcitabine, hydroxyurea, idarubicin, ifosfamide, irinotecan, lomustine, mechlorethamine, melphalan, mercaptopurine, methotrxate, mitomycin, mitoxantrone, oxaliplatin, paclitaxel, pemetrexed, procarbazine, streptozocin, tafluposide, temozolomide, teniposide, tioguanine, topotecan, uramustine, valrubicin, vinblastine, vincristine, vindesine, and vinorelbine.

In various methods described herein, platelets are loaded with one or more any of a variety of drugs. In some embodiments, platelets are loaded with a protein (e.g., an antibody or antibody conjugate). For example, platelets can be loaded with one or more of cetuximab (ERBITUX®), necitumumab (PORTRAZZA™, IMC-11F8), panitumumab (ABX-EGF, VECTIBIX®), matuzumab (EMD-7200), nimotuzumab (h-R3, BIOMAb EGFR®), zalutumab, MDX447, OTSA 101, OTSA101-DTPA-90Y, ABBV-399, depatuxizumab (humanized mAb 806, ABT-806), depatuxizumab mafodotin (ABT-414), SAIT301, Sym004, MAb-425, Modotuximab (TAB-H49), futuximab (992 DS), zalutumumab, Sym013, AMG 595, JNJ-61186372, LY3164530, IMGN289, KL-140, R05083945, SCT200, CPGJ602, GP369, BAY1187982, FPA144 (bemarituzumab), bevacizumab (AVASTIN®), ranibizumab, trastuzumab (HERCEPTIN®), pertuzumab (PERJETA®), trastuzumab-dkst (OGIVRI®), ado-trastuzumab emtansine (KADCYLA®, T-DM1), Zemab, DS-8201a, MFGR1877S, B-701, rilotumumab (AMG102), ficlatuzumab (AV-299), FP-1039 (GSK230), TAK701, YYB101, onartuzumab (MetMAb), ipilimumab (YERVOY®), tremelimumab (CP-675,206), pembrolizumab (KEYTRUDA®), nivolumab (OPDIVO®), atezolizumab (TECENTRIQ®), avelumab (BAVENCIO®), durvalumab (IMFINZI™), BIOO-1, BIOO-2, and BIOO-3.

In various methods described herein, platelets are loaded with one or more any of a variety of drugs. In some embodiments, platelets are loaded with an oligopeptide. For example, platelets can be loaded with one or more of RGD-SSL-Dox, LPD-PEG-NGR, PNC-2, PNC-7, RGD-PEG-Suc-PD0325901, VWCS, FWCS, p16, Bac-7-ELP-p21, Pen-ELP-p21, TAT-Bim, Poropeptide-Bax, R8-Bax, CT20p-NP, RRM-MV, RRM-IL12, PNC-27, PNC-21, PNC-28, Tat-aHDM2, Int-H1-S6A, F8A, Pen-ELP-H1, BACl-ELP-H1, goserelin, leuprolide, Buserelin, Triptorelin, Degarelix, Pituitary adenylate cyclase activating peptide (PACAP), cilengitide, ATN-161 (AcPHSCN-NH2), TTK, LY6K, IMP-3, P16_37-63, VEGFR1-A24-1084, uMMP-2, uTIMP-1, MIC-1/GDF15, RGS6, LGR5, PGI/II, CA242, EN2, UCP2, a HER-2 peptide, MUClm, HNP1-3, L-glutamine L-tryptophan (IM862), CPAA-783-EPPT1, serum C-peptide, WT1, KIF20A, GV1001, LY6K-177, PAP-114-128, E75, SU18, SU22, ANP, TCP-1, F56, WT1, TERT572Y, disruptin, TREM-1, LFC131, BPP, TH10, BC71, RC-3095, RC-3940-II, RC-3950, (KLAKLAK)2, RGD-(KLAKLAK)2, NGR-(KLAKLAK)2, and SAH-8 (stapled peptides).

In various methods described herein, platelets are loaded with one or more any of a variety of drugs. In some embodiments, platelets are loaded with a non-miRNA nucleic acid, a non-siRNA nucleic acid, and a non-mRNA nucleic acid (e.g., non-miRNA, DNA, other naturally or non-naturally occurring nucleic acids, and polymers thereof), including various modifications thereof). For example, platelets can be loaded with one or more of SPC2996, SIRNAPLUS, ALN-HTT, ISIS-199044, custirsen (OGX-011, ISI-112989, TV-1011), ISIS-AR-2.5RX (ISIS-ARRx, AZD-5312, ISIS-AZ1Rx, ISIS-560131), ISIS-STAT3-2.5Rx (ISIS-STAT3-2.5Rx, ISIS-481464, AZD-9150), BP-100-1.01, NOX-A12 (olaptesed peqol), PNT-2258, ATL-1103, RX-0201, ACT-GRO-777, litenimod, trabedersen (AP-2009), IMO-2055, OHR-118, imetelstat, GNKG-168, RG-6061, SPC-3042, STAT3 decoys, an anti-CD22 antibody-MXD3 antisense oligonucleotide conjugate, AST-008, ASncmtRNA, an EGFRAS GPNA, ASPH-1047 (ASPH-0047), STICKY SIRNA, aganirsen, BO-110, NOX-593, Adva-R46, EZN-4482, EZN-4496, EZN-3889, EZN-3892, EZN-4150, IMO-2125 (HYB-2125), OGX-225, ATL-1101, aqatolimod, AGX-1053, AEG-35156, qataparsen, ISS-1018, CpG-1826 (ODN-1826), CpG-2216 (ODN-2216), CpG-2395, oblimersen, pbi-shRNAK-ras LP, LNA anti-miR-155, ISIS-20408, ISIS-199044, AP-11014, NOX-A50, beclanorsen, ISIS-345794, ISIS-15421, GRO-29A, LOR-2501 (GTI-2501), ISIS-7597, ISIS-3466, ISIS-2503, and GEM-231.

In various methods described herein, platelets are loaded with one or more any of a variety of drugs. In some embodiments, platelets are loaded with an aptamer. For example, platelets can be loaded with one or more of ARC126 (RNA), AX102 (RNA), SL (2)-B (DNA), RNV66 (DNA), AS1411 (DNA), FCL-II (DNA, modified form AS1411), NOX-A12 (RNA), E0727 (RNA), CL428 (RNA), KDI130 (RNA), TuTu2231 (RNA), Trimeric apt (DNA), PNDA-3 (DNA), TTA140,41 (DNA), GBI-1042 (DNA), NAS-24 (DNA), YJ-1 (RNA), AGE-apt (DNA), A-P50 (RNA), GL21.T (RNA), OPN-R3 (RNA), AGC03 (DNA), cy-apt (DNA), BC15 (DNA), A9g (RNA), ESTA (DNA), M12-23 (RNA), OX40-apt (RNA), De160 (RNA), PSMA-4-1BB-apt (RNA), CD16a/c-Met-apt (RNA), VEGF-4-1BB apt (DNA), MP7 (DNA), aptPD-L1 (DNA), R5A1 (RNA), CL-42 (RNA), CD44-EpCAM aptamer (RNA), TIM3Apt (RNA), CD40apt (RNA), AptCTLA-4 (DNA), AON-D21 1-Aptamer (RNA/DNA), and BN-210.

In some embodiments, a drug loaded into platelets is modified. For example, a drug can be modified to increase its stability during the platelet loading process, while the drug is loaded into the platelet, and/or after the drug's release from a platelet. In some embodiments, the modified drug's stability is increased with little or no adverse effect on its activity. For example, the modified drug can have at least 70%, 75%, 80%, 85%, 90%, 95%, 96%, 97%, 98%, 99% or more of the activity of the corresponding unmodified drug. In some embodiments, the modified drug has 100% (or more) of the activity of the corresponding unmodified drug. Various modifications that stabilize drugs are known in the art. In some embodiments, the drug is a nucleic acid, which nucleic acid is stabilized by one or more of a stabilizing oligonucleotide (see, e.g., U.S. Application Publication No. 2018/0311176), a backbone/side chain modification (e.g., a 2-sugar modification such as a 2′-fluor, methoxy, or amine substitution, or a 2′-thio (—SH), 2′-azido (—N3), or 2′-hydroxymethyl (—CH2OH) modification), an unnatural nucleic acid substitution (e.g., an S-glycerol, cyclohexenyl, and/or threose nucleic acid substitution, an L-nucleic acid substitution, a locked nucleic acid (LNA) modification (e.g., the ribose moiety of an LNA nucleotide is modified with an extra bridge connecting the 2′ oxygen and 4′ carbon), conjugation with PEG, a nucleic acid bond modification or replacement (e.g., a phosphorothioate bond, a methylphosphonate bond, or a phosphorodiamidate bond), a reagent or reagents (e.g., intercalating agents such as coralyne, neomycin, and ellipticine; also see US Publication Application Nos. 2018/0312903 and 2017/0198335, each of which are incorporated herein by reference in their entireties, for further examples of stabilizing reagents). In some embodiments, the drug is a polypeptide, which polypeptide is stabilized by one or more of cyclization of the peptide sequence [e.g., between side chains or ends of the peptide sequence (for example, head to tail, N-backbone to N-backbone, end to N-backbone, end to side chain, side chain to N-backbone, side chain to side chain) through disulfide, lanthionine, dicarba, hydrazine, or lactam bridges], a backbone/side chain modification, an unnatural residue substitution (e.g., a D-amino acid, an N-methyl-α-amino acid, a non-proteogenic constrained amino acid or a β-amino acid), a peptide bond modification or replacement [e.g., NH-amide alkylation, the carbonyl function of the peptide bond can be replaced by CH2 (reduced bond: —CH2-NH—), C(═S) (endothiopeptide, —C(═S)—NH—) or PO2H (phosphonamide, —P(═O)OH—NH—), the NH-amide bond can be exchanged by O (depsipeptide, —CO—O—), S (thioester, —CO—S—) or CH2 (ketomethylene, —CO—CH2-), a retro-inverso bond (—NH—CO—), a methylene-oxy bond (—CH2-), a thiomethylene bond (—CH2-S—), a carba bond (—CH2-CH2-), and a hydroxyethylene bond (—CHOH—CH2-)], a disulfide-bridged conjugation with synthetic aromatics (see e.g., Chen et al. Org Biomol Chem. 2017, 15(8):1921-1929, which is incorporated by reference herein in its entirety), blocking N- or C-terminal ends of the peptide (e.g., by N-acylation, N-pyroglutamate, or C-amidation or the addition of carbohydrate chains through, for example, glycosylation with glucose, xylose, hexose), an N-terminal esterification (phosphoester), a pegylation modification, and a reagent or reagents (see, e.g., US Publication Application No. 2017/0198335). See. e.g., Vlieghe et al. Drug Discovery Today, 2010, 15, 40-56, which is incorporated by reference herein in its entirety.

In some embodiments, a drug loaded into platelets is modified to include an imaging agent. For example, a drug can be modified with an imaging agent in order to image the drug loaded platelet in vivo. In some embodiments, a drug can be modified with two or more imaging agents (e.g., any two or more of the imaging agents described herein). In some embodiments, a drug loaded into platelets is modified with a radioactive metal ion, a paramagnetic metal ion, a gamma-emitting radioactive halogen, a positron-emitting radioactive non-metal, a hyperpolarized NMR-active nucleus, a reporter suitable for in vivo optical imaging, or a beta-emitter suitable for intravascular detection. For example, a radioactive metal ion can include, but is not limited to, positron emitters such as ⁵⁴Cu, ⁴⁸V, ⁵²Fe, ⁵⁵Co, ⁹⁴Tc or ⁶⁸Ga; or gamma-emitters such as ¹⁷¹Tc, ¹¹¹In, ¹¹³In, or ⁶⁷Ga. For example, a paramagnetic metal ion can include, but is not limited to Gd(III), a Mn(II), a Cu(II), a Cr(III), a Fe(III), a Co(II), a Er(II), a Ni(II), a Eu(III) or a Dy(III), an element comprising an Fe element, a neodymium iron oxide (NdFeO3) or a dysprosium iron oxide (DyFeO3). For example, a paramagnetic metal ion can be chelated to a polypeptide or a monocrystalline nanoparticle. For example, a gamma-emitting radioactive halogen can include, but is not limited to ¹²³I, ¹³¹I or ⁷⁷Br. For example, a positron-emitting radioactive non-metal can include, but is not limited to ¹¹C, ¹³N, ¹⁵O, ¹⁷F, ¹⁸F, ⁷⁵Br, ⁷⁶Br or 1241 For example, a hyperpolarized NMR-active nucleus can include, but is not limited to ¹³C, ¹⁵N, ¹⁹F, ²⁹Si and ³¹P. For example, a reporter suitable for in vivo optical imaging can include, but is not limited to any moiety capable of detection either directly or indirectly in an optical imaging procedure. For example, the reporter suitable for in vivo optical imaging can be a light scatterer (e.g., a colored or uncolored particle), a light absorber or a light emitter. For example, the reporter can be any reporter that interacts with light in the electromagnetic spectrum with wavelengths from the ultraviolet to the near infrared. For example, organic chromophoric and fluorophoric reporters include groups having an extensive delocalized electron system, e.g. cyanines, merocyanines, indocyanines, phthalocyanines, naphthalocyanines, triphenylmethines, porphyrins, pyrilium dyes, thiapyrilium dyes, squarylium dyes, croconium dyes, azulenium dyes, indoanilines, benzophenoxazinium dyes, benzothiaphenothiazinium dyes, anthraquinones, napthoquinones, indathrenes, phthaloylacridones, trisphenoquinones, azo dyes, intramolecular and intermolecular charge-transfer dyes and dye complexes, tropones, tetrazines, b/s(dithiolene) complexes, bis(benzene-dithiolate) complexes, iodoaniline dyes, b/stS.O-dithiolene) complexes. For example, the reporter can be, but is not limited to a fluorescent, a bioluminescent, or chemiluminescent polypeptide. For example, a fluorescent or chemiluminescent polypeptide is a green florescent protein (GFP), a modified GFP to have different absorption/emission properties, a luciferase, an aequorin, an obelin, a mnemiopsin, a berovin, or a phenanthridinium ester. For example, a reporter can be, but is not limited to rare earth metals (e.g., europium, samarium, terbium, or dysprosium), or fluorescent nanocrystals (e.g., quantum dots). For example, a reporter may be a chromophore that can include, but is not limited to fluorescein, sulforhodamine 101 (Texas Red), rhodamine B, rhodamine 6G, rhodamine 19, indocyanine green, Cy2, Cy3, Cy3.5, Cy5, Cy5.5, Cy7, Marina Blue, Pacific Blue, Oregon Green 88, Oregon Green 514, tetramethylrhodamine, and Alexa Fluor 350, Alexa Fluor 430, Alexa Fluor 532, Alexa Fluor 546, Alexa Fluor 555, Alexa Fluor 568, Alexa Fluor 594, Alexa Fluor 633, Alexa Fluor 647, Alexa Fluor 660, Alexa Fluor 680, Alexa Fluor 700, and Alexa Fluor 750. For example, a beta-emitter can include, but is not limited to radio metals ⁶⁷Cu, ⁸⁹Sr, ⁹⁰Y ¹⁵³Sm, ¹⁸⁵Re, ¹⁸⁸Re or ¹⁹²Ir, and non-metals ³²P, ³³P, ³⁸S, ³⁸Cl, ³⁹Cl, ⁸²Br and ⁸³Br. In some embodiments, a drug loaded into platelets can be associated with gold or other equivalent metal particles (such as nanoparticles). For example, a metal particle system can include, but is not limited to gold nanoparticles (e.g., Nanogold™).

In some embodiments, a drug loaded into platelets that is modified with an imaging agent is imaged using an imaging unit. The imaging unit can be configured to image the drug loaded platelets in vivo based on an expected property (e.g., optical property from the imaging agent) to be characterized. For example, imaging techniques (in vivo imaging using an imaging unit) that can be used, but are not limited to are: computer assisted tomography (CAT), magnetic resonance spectroscopy (MRS), magnetic resonance imaging (MRI), positron emission tomography (PET), single-photon emission computed tomography (SPECT), or bioluminescence imaging (BLI). Chen Z., et. al., Advance of Molecular Imaging Technology and Targeted Imaging Agent in Imaging and Therapy, Biomed Res Int., February 13, doi: 10.1155/2014/819324 (2014) have described various imaging techniques and which is incorporated by reference herein in its entirety.

In some embodiments, such as embodiments wherein the platelets are treated with the drug and the buffer sequentially as disclosed herein, the drug may be loaded in a liquid medium that may be modified to change the proportion of media components or to exchange components for similar products, or to add components necessary for a given application.

In some embodiments the loading buffer, and/or the liquid medium, may comprise one or more of a) water or a saline solution, b) one or more additional salts, or c) a base. In some embodiments, the loading buffer, and/or the liquid medium, may comprise one or more of a) DMSO, b) one or more salts, or c) a base.

In some embodiments the loading agent is loaded into the platelets in the presence of an aqueous medium. In some embodiments the loading agent is loaded in the presence of a medium comprising DMSO. As an example, one embodiment of the methods herein comprises treating platelets with a drug and with an aqueous loading buffer comprising a salt, a base, a loading agent, and optionally at least one organic solvent, to form the cargo-loaded platelets. As an example, one embodiment of the methods herein comprises treating platelets with a drug and with a loading buffer comprising DMSO and comprising a salt, a base, a loading agent, and optionally ethanol, to form the cargo-loaded platelets.

In some embodiments the loading buffer, and/or the liquid medium, may comprise one or more salts selected from phosphate salts, sodium salts, potassium salts, calcium salts, magnesium salts, and any other salt that can be found in blood or blood products, or that is known to be useful in drying platelets, or any combination of two or more of these.

Preferably, these salts are present in the composition at an amount that is about the same as is found in whole blood.

In some embodiments, the drug-loaded platelets are prepared by incubating the platelets with the drug in the liquid medium for different durations at or at different temperatures from 15-45° C., or about 37° C. (cell to drug volume ratio of 1:2).

In some embodiments, the platelets form a suspension in a liquid medium at a concentration from 10,000 platelets/μL to 10,000,000 platelets/μL, such as 50,000 platelets/μL to 2,000,000 platelets/μL, such as 100,000 platelets/μL to 500,000 platelets/μL, such as 150,000 platelets/μL to 300,000 platelets/μL, such as 200,000 platelets/μL.

In some embodiments, one or more other components may be loaded in the platelets. In some embodiments, the one or more other components may be loaded concurrently with the drug. In some embodiments, the one or more other components and the drug may be loaded sequentially in either order. Components may include an agent (e.g., an anti-aggregation agent) that reduces or prevents platelet aggregation and activation during the loading process. Exemplary components (e.g., anti-aggregation agents) may include an anti-aggregation agent such as, Prostaglandin E1 or Prostacyclin and or EDTA/EGTA to prevent platelet aggregation and activation during the loading process. Additional non-limiting anti-aggregation agents may include, GR144053, FR171113, aspirin, MeSADP, PSB 0739, Cangrelor, Tirofiban (e.g., Aggrastat™), and MitoTEMPO, N-acetyyl-L-cysteine, cytcochalasin D, Staurosporine, Mepacrine, actezolamide, or dichloroacetate. These components may be used alone or in combination with one another.

Accordingly, in some embodiments, an agent suitable for treatment of cancer, such as doxorubicin, may be loaded together with, prior to, or following, a GPIIb/IIIa inhibitor. In some embodiments, the cancer is acute lymphoblastic leukemia. In some embodiments, the cancer is acute myeloid leukemia. In some embodiments, the cancer is breast cancer or metastasized breast cancer. In some embodiments, the cancer is gastric cancer. In some embodiments, the cancer is Hodgkin lymphoma. In some embodiments, the cancer is neuroblastoma. In some embodiments, the cancer is Non-Hodgkin lymphoma. In some embodiments, the cancer is ovarian cancer. In some embodiments, the cancer is small cell lung cancer. In some embodiments, the cancer is soft tissue and bone sarcomas. In some embodiments, the cancer is thyroid cancer. In some embodiments, the cancer is transitional cell bladder cancer. In some embodiments, the cancer is Wilms tumor.

Accordingly, in some embodiments, an agent suitable for treatment of cancer, such as olaparib (also known as AZD-2281, MK-7339, trade name Lynparza®), may be loaded together with, prior to, or following, a GPIIb/IIIa inhibitor. In some embodiments, the cancer is ovarian cancer. In some embodiments, the cancer is breast cancer.

Accordingly, in some embodiments, an agent suitable for treatment of cancer, such as paclitaxel (Taxol®), may be loaded together with, prior to, or following, a GPIIb/IIIa inhibitor. In some embodiments, the cancer is Kaposi sarcoma. In some embodiments, the cancer is breast cancer. In some embodiments, the cancer is non-small cell lung cancer. In some embodiments, the cancer is ovarian cancer.

In some embodiments, an agent suitable for treatment of cancer, such as doxorubicin, may be loaded together with, prior to, or following, P2Y1 receptor activation inhibitor, a P2Y1 agonist, P2Y12 agonist, a P2Y13 agonist, a PAR 1 antagonist, a COX inhibitor, a P2Y12 inhibitor, a thiol supplement, a ROS antagonist, an actin polymerization inhibitor, protein kinase C inhibitor, phospholipase A2 inhibitor, Rho kinase inhibitor, a carbonic anhydrase inhibitor, or a PDK inhibitor.

In some embodiments, the one or more other components that are loaded in the platelets comprise Prostaglandin E1 (PGE1) or Prostacyclin.

In some embodiments, the one or more other components that are loaded in the platelets do not comprise Prostaglandin E1 or Prostacyclin.

In some embodiments, the one or more other components that are loaded in the platelets comprise EGTA.

In some embodiments, the one or more other components that are loaded in the platelets do not comprise EGTA.

In some embodiments, the one or more other components that are loaded in the platelets comprise EDTA.

In some embodiments, the one or more other components that are loaded in the platelets do not comprise EDTA.

The table below shows the effect of the addition of antiplatelet compounds on DOX-induced platelet aggregation:

TABLE A

| | | | | | | Max Platelet Count | | | |
|---|---|---|---|---|---|---|---|---|---|
| | | | | | | (Normalized to Untreated) | | | |
| | | | | Recommended | | 0.5 mg/mL | 1 mg/mL | 0.6 mM | 1.2 mM |
| # | Reagent | Target | Source | concentration | | DOX | DOX | DOX | DOX |
| 1 | GR144053 | GPIIb/IIIa | PMID: 9700979 | IC50 = 37 nM | | 83% | 42% | N/A | N/A |
| | | inhibitor | | | | | | | |
| 2 | PGE1 | P2Y1 | PMID: 22385219 | 22 nM-1 μM | | N/A | N/A | N/A | N/A |
| | | receptor | | | | | | | |
| | | activation | PMID: 22268418 | | | | | | |
| | | Inhibitor | | | | | | | |
| 3 | MeSADP | P2Y1, | PMID: 9442039 | 10 | μM | 33% | 10% | N/A | N/A |
| | | P2Y12, and | | | | | | | |
| | | P2Y13 | | | | | | | |
| | | Agonist | | | | | | | |
| 4 | FR171113 | PAR1 | PMID: 10611442 | 0.3 | μM | 51% | 16% | N/A | N/A |
| | | antagonist | | | | | | | |
| 5 | Aspirin (ASA) | COX | PMID: 3370916 | 40-500 | μM | 52% | 15% | N/A | N/A |
| | | inhibitor | | | | | | | |
| 6 | Cangrelor | P2Y12 | PMID: 23236426 | 1 | μM | 47% | 26% | N/A | N/A |
| | | Inhibitor | | | | | | | |
| 7 | PSB 0739 | P2Y12 | PMID: 27695417 | 500 | nM | 26% | 18% | N/A | N/A |
| | | Inhibitor | | | | | | | |
| 8 | N- | Thiol | PMID: 19426282 | 5 | mM | N/A | N/A | 73% | N/A |
| | Acetylcysteine | Supplement | page 1179 | | | | | | |
| 9 | MitoTEMPO | ROS | PMID: 25988386 | 10 | μM | N/A | N/A | N/A | 37% |
| | | Antagonist | methods 4.3 | | | | | | |
| 10 | Tirofiban | GPIIb/IIIa | PMID: 11406724 | 5 | μM | N/A | N/A | 73% | N/A |
| | | inhibitor | abstract | | | | | | |

The table below shows alternatively proposed antiplatelet compounds to combat DOX-induced platelet aggregation:

TABLE B

| | | | | Recommended | |
|---|---|---|---|---|---|
| # | Reagent | Target | Source | concentration | |
| 1 | Cytochalasin D | actin | PMID: 10682859 | 10 | μM |
| | | polymerization | page 357 | | |
| 2 | Staurosporine | protein | PMID: 10051374 | 25 | nM |
| | | kinase C | methods “PKC studies” | to | |
| | | inhibitor | PMID: 11895774 | 10 | μM |
| | | | (function or reagent) | | |
| 3 | Mepacrine | Phospholipase | PMID: 3931692 | 2.5-20 | μM |

A2 inhibitor

In some embodiments, other components may include imaging agents. For example, an imaging agent can include, but is not limited to a radioactive metal ion, a paramagnetic metal ion, a gamma-emitting radioactive halogen, a positron-emitting radioactive non-metal, a hyperpolarized NMR-active nucleus, a reporter suitable for in vivo optical imaging, or a beta-emitter suitable for intravascular detection. For example, a radioactive metal ion can include, but is not limited to, positron emitters such as 54Cu, 48V, 52Fe, 55Co, 94Tc or 68Ga; or gamma-emitters such as 171Tc, 111In, 113In, or 67Ga. For example, a paramagnetic metal ion can include, but is not limited to Gd(III), a Mn(II), a Cu(II), a Cr(III), a Fe(III), a Co(II), a Er(II), a Ni(II), a Eu(III) or a Dy(III), an element comprising an Fe element, a neodymium iron oxide (NdFeO3) or a dysprosium iron oxide (DyFeO3). For example, a paramagnetic metal ion can be chelated to a polypeptide or a monocrystalline nanoparticle. For example, a gamma-emitting radioactive halogen can include, but is not limited to 1231, 131I or 77Br. For example, a positron-emitting radioactive non-metal can include, but is not limited to 11C, 13N, 150, 17F, 18F, 75Br, 76Br or 1241. For example, a hyperpolarized NMR-active nucleus can include, but is not limited to 13C, 15N, 19F, 29Si and 31P. For example, a reporter suitable for in vivo optical imaging can include, but is not limited to any moiety capable of detection either directly or indirectly in an optical imaging procedure. For example, the reporter suitable for in vivo optical imaging can be a light scatterer (e.g., a colored or uncolored particle), a light absorber or a light emitter. For example, the reporter can be any reporter that interacts with light in the electromagnetic spectrum with wavelengths from the ultraviolet to the near infrared. For example, organic chromophoric and fluorophoric reporters include groups having an extensive delocalized electron system, e.g. cyanines, merocyanines, indocyanines, phthalocyanines, naphthalocyanines, triphenylmethines, porphyrins, pyrilium dyes, thiapyrilium dyes, squarylium dyes, croconium dyes, azulenium dyes, indoanilines, benzophenoxazinium dyes, benzothiaphenothiazinium dyes, anthraquinones, napthoquinones, indathrenes, phthaloylacridones, trisphenoquinones, azo dyes, intramolecular and intermolecular charge-transfer dyes and dye complexes, tropones, tetrazines, b/s(dithiolene) complexes, bis(benzene-dithiolate) complexes, iodoaniline dyes, b/stS.O-dithiolene) complexes. For example, the reporter can be, but is not limited to a fluorescent, a bioluminescent, or chemiluminescent polypeptide. For example, a fluorescent or chemiluminescent polypeptide is a green florescent protein (GFP), a modified GFP to have different absorption/emission properties, a luciferase, an aequorin, an obelin, a mnemiopsin, a berovin, or a phenanthridinium ester. For example, a reporter can be, but is not limited to rare earth metals (e.g., europium, samarium, terbium, or dysprosium), or fluorescent nanocrystals (e.g., quantum dots). For example, a reporter may be a chromophore that can include, but is not limited to fluorescein, sulforhodamine 101 (Texas Red), rhodamine B, rhodamine 6G, rhodamine 19, indocyanine green, Cy2, Cy3, Cy3.5, Cy5, Cy5.5, Cy7, Marina Blue, Pacific Blue, Oregon Green 88, Oregon Green 514, tetramethylrhodamine, and Alexa Fluor 350, Alexa Fluor 430, Alexa Fluor 532, Alexa Fluor 546, Alexa Fluor 555, Alexa Fluor 568, Alexa Fluor 594, Alexa Fluor 633, Alexa Fluor 647, Alexa Fluor 660, Alexa Fluor 680, Alexa Fluor 700, and Alexa Fluor 750. For example, a beta-emitter can include, but is not limited to radio metals 67Cu, 89Sr, 90Y, 153Sm, 185Re, 188Re or 192Ir, and non-metals 32P, 33P, 38S, 38Cl, 39Cl, 82Br and 83Br. In some embodiments, a drug loaded into platelets can be associated with gold or other equivalent metal particles (such as nanoparticles). For example, a metal particle system can include, but is not limited to gold nanoparticles (e.g., Nanogold™).

In some embodiments, the one or more imaging agents loaded concurrently with a drug is imaged using an imaging unit. The imaging unit can be configured to image the drug loaded platelets in vivo based on an expected property (e.g., optical property from the imaging agent) to be characterized. For example, imaging techniques (in vivo imaging using an imaging unit) that can be used, but are not limited to are: computer assisted tomography (CAT), magnetic resonance spectroscopy (MRS), magnetic resonance imaging (MRI), positron emission tomography (PET), single-photon emission computed tomography (SPECT), or bioluminescence imaging (BLI).

Chen Z. et. al., (2014) have described various imaging techniques and which is incorporated by reference herein in its entirety.

In some embodiments, the drug-loaded platelets are prepared by incubating the platelets with the drug in the liquid medium for different durations. The step of incubating the platelets to load one or more cargo, such as a drug(s), includes incubating the platelets for a time suitable for loading, as long as the time, taken in conjunction with the temperature, is sufficient for the drug to come into contact with the platelets and, preferably, be incorporated, at least to some extent, into the platelets. For example, in some embodiments, the drug-loaded platelets are prepared by incubating the platelets with the drug in the liquid medium for at least about 5 minutes (mins) (e.g., at least about 20 mins, about 30 mins, about 1 hour (hr), about 2 hrs, about 3 hrs, about 4 hrs, about 5 hrs, about 6 hrs, about 7 hrs, about 8 hrs, about 9 hrs, about 10 hrs, about 12 hrs, about 16 hrs, about 20 hrs, about 24 hrs, about 30 hrs, about 36 hrs, about 42 hrs, about 48 hrs, or at least about 48 hrs. In some embodiments, the drug-loaded platelets are prepared by incubating the platelets with the drug in the liquid medium for no more than about 48 hrs (e.g., no more than about 20 mins, about 30 mins, about 1 hour (hr), about 2 hrs, about 3 hrs, about 4 hrs, about 5 hrs, about 6 hrs, about 7 hrs, about 8 hrs, about 9 hrs, about 10 hrs, about 12 hrs, about 16 hrs, about 20 hrs, about 24 hrs, about 30 hrs, about 36 hrs, or no more than about 42 hrs). In some embodiments, the drug-loaded platelets are prepared by incubating the platelets with the drug in the liquid medium from about 10 mins to about 48 hours (e.g., from about 20 mins to about 36 hrs, from about 30 mins to about 24 hrs, from about 1 hr to about 20 hrs, from about 2 hrs to about 16 hours, from about 10 mins to about 24 hours, from about 20 mins to about 12 hours, from about 30 mins to about 10 hrs, or from about 1 hr to about 6 hrs. In one embodiment, treating platelets, platelet derivatives, or thrombosomes with a drug, a liquid medium, a buffer comprising a salt, a base, a loading agent, and optionally at least one organic solvent, and/or with any loading protocol described herein to form the drug-loaded platelets comprises contacting the platelets, platelet derivatives, or thrombosomes with a drug, a liquid medium, a buffer comprising a salt, a base, a loading agent, and optionally at least one organic solvent and/or with any loading protocol described herein for a period of time, such as a period of 5 minutes to 48 hours, such as 2 hours.

In some embodiments, the drug-loaded platelets are prepared by incubating the platelets with the drug in the liquid medium at different temperatures. The step of incubating the platelets to load one or more cargo, such as a drug(s), includes incubating the platelets with the drug in the liquid medium at a temperature that, when selected in conjunction with the amount of time allotted for loading, is suitable for loading. In general, the platelets with the drug in the liquid medium are incubated at a suitable temperature (e.g., a temperature above freezing) for at least a sufficient time for the drug to come into contact with the platelets. In embodiments, incubation is conducted at 37° C. In certain embodiments, incubation is performed at 4° C. to 45° C., such as 15° C. to 42° C. For example, in embodiments, incubation is performed at 35° C. to 40° C. (e.g., 37° C.) for 110 to 130 (e.g., 120) minutes and for as long as 24-48 hours.

In some embodiments of a method of preparing drug-loaded platelets disclosed herein, the method further comprises acidifying the platelets, or pooled platelets, to a pH of about 6.0 to about 7.4, prior to a treating step disclosed herein. In some embodiments, the method comprises acidifying the platelets to a pH of about 6.5 to about 6.9. In some embodiments, the method comprises acidifying the platelets to a pH of about 6.6 to about 6.8. In some embodiments, the acidifying comprises adding to the pooled platelets a solution comprising Acid Citrate Dextrose.

In some embodiments, the platelets are isolated prior to a treating step. In some embodiments, the method further comprises isolating platelets by using centrifugation. In some embodiments, the centrifugation occurs at a relative centrifugal force (RCF) of about 800 g to about 2000 g. In some embodiments, the centrifugation occurs at relative centrifugal force (RCF) of about 1300 g to about 1800 g. In some embodiments, the centrifugation occurs at relative centrifugal force (RCF) of about 1500 g. In some embodiments, the centrifugation occurs for about 1 minute to about 60 minutes. In some embodiments, the centrifugation occurs for about 10 minutes to about 30 minutes. In some embodiments, the centrifugation occurs for about 30 minutes.

In some embodiments, the platelets are at a concentration from about 2,000 platelets/μl to about 500,000,000 platelets/μl. In some embodiments, the platelets are at a concentration from about 50,000 platelets/μl to about 4,000,000 platelets/μl. In some embodiments, the platelets are at a concentration from about 100,000 platelets/μl to about 300,000,000 platelets/μl. In some embodiments, the platelets are at a concentration from about 1,000,000 to about 2,000,000. In some embodiments, the platelets are at a concentration of about 200,000,000 platelets/μl.

In some embodiments, the buffer is a loading buffer comprising the components as listed in Table 1 herein. In some embodiments, the loading buffer comprises one or more salts, such as phosphate salts, sodium salts, potassium salts, calcium salts, magnesium salts, and any other salt that can be found in blood or blood products. Exemplary salts include sodium chloride (NaCl), potassium chloride (KCl), and combinations thereof. In some embodiments, the loading buffer includes from about 0.5 mM to about 100 mM of the one or more salts. In some embodiments, the loading buffer includes from about 1 mM to about 100 mM (e.g., about 2 mM to about 90 mM, about 2 mM to about 6 mM, about 50 mM to about 100 mM, about 60 mM to about 90 mM, about 70 to about 85 mM) about of the one or more salts. In some embodiments, the loading buffer includes about 5 mM, about 75 mM, or about 80 mM of the one or more salts.

In some embodiments, the loading buffer includes one or more buffers, e.g., N-2-hydroxyethylpiperazine-N′-2-ethanesulfonic acid (HEPES), or sodium-bicarbonate (NaHCO₃). In some embodiments, the loading buffer includes from about 5 to about 100 mM of the one or more buffers. In some embodiments, the loading buffer includes from about 5 to about 50 mM (e.g., from about 5 mM to about 40 mM, from about 8 mM to about 30 mM, about 10 mM to about 25 mM) about of the one or more buffers. In some embodiments, the loading buffer includes about 10 mM, about 20 mM, about 25 mM, or about 30 mM of the one or more buffers.

In some embodiments, the loading buffer includes one or more saccharides, such as monosaccharides and disaccharides, including sucrose, maltose, trehalose, glucose, mannose, dextrose, and xylose. In some embodiments, the loading buffer includes from about 10 mM to about 1,000 mM of the one or more saccharides. In some embodiments, the loading buffer includes from about 50 to about 500 mM of the one or more saccharides. In embodiments, one or more saccharides is present in an amount of from 10 mM 10 to 500 mM. In some embodiments, one or more saccharides is present in an amount of from 50 mM to 200 mM. In embodiments, one or more saccharides is present in an amount from 100 mM to 150 mM.

In some embodiments, the loading buffer includes adding an organic solvent, such as ethanol, to the loading solution. In such a loading buffer, the solvent can range from about 0.1% (v/v) to about 5.0% (v/v), such as from about 0.3% (v/v) to about 3.0% (v/v), or from about 0.5% (v/v) to about 2% (v/v).

In some embodiments, the drug comprises doxorubicin (“DOX”). DOX interacts with DNA by intercalation and inhibits macromolecular biosynthesis (Tacar, O. et. al., Doxorubicin: an update on anticancer molecular action, toxicity and novel drug delivery systems, The Journal of Pharmacy and Pharmacology, 65 (2): 157-70. doi:10.1111/j.2042-7158.2012.01567.x. PMID 23278683 (2013), which is incorporated herein by reference).

In some embodiment, the drug comprises paclitaxel. In some embodiment, the drug comprises paclitaxel that is not in the presence of Cremophor EL. In some embodiments, the drug comprises paclitaxel that is not in the presence (e.g., an excipient) of a polyexthoxylated castor oil. For example, paclitaxel that is not in the presence of an excipient comprising a polyethylene glycol ether.

In some embodiments, the drug comprises a poly ADP ribose polymerase (PARP) inhibitor (PARPi). PARPis prevent the normal repair of DNA breaks which in turn leads to cell death. In some embodiments, the PARPi is olaparib.

In some embodiments, the method further comprises incubating the drug in the presence of the loading buffer prior to the treatment step. In some embodiments, the method further comprises incubating the loading buffer and a solution comprising the drug and water at about 37° C. using different incubation periods. In some embodiments, the solution includes a concentration of about 1 nM to about 1000 mM of the drug. In some embodiments, the solution includes a concentration of about 10 nM to about 10 mM of the drug. In some embodiments, the solution includes a concentration of about 100 nM to 1 mM of the drug. In some embodiments, the solution includes a concentration of from about 10 mg/ml of water to about 100 mg/ml. In some embodiments, the solution includes a concentration of from about 20 mg/ml of water to about 80 mg/ml. In some embodiments, the solution includes a concentration of from about 40 mg/ml of water to about 60 mg/ml. In some embodiments, the incubation of the drug in the presence of the loading buffer is performed from about 1 minute to about 2 hours. In some embodiments, the incubation is performed at an incubation period of from about 5 minutes to about 1 hour. In some embodiments, the incubation is performed at an incubation period of from about 10 minutes to about 30 minutes. In some embodiments, the incubation is performed at an incubation period of about 20 minutes.

In some embodiments, the method further comprises mixing the platelets and the drug in the presence of the loading buffer at 37° C., using a platelet to drug volume ratio of 1:2. In some embodiments, the method further comprises incubating the platelets and the drug in the presence of the loading buffer at 37° C. using a platelet to drug volume ratio of 1:2, using different 55N incubation periods. In some embodiments, the incubation is performed at an incubation period of from about 5 minutes to about 12 hours. In some embodiments, the incubation is performed at an incubation period of from about 10 minutes to about 6 hours. In some embodiments, the incubation is performed at an incubation period of from about 15 minutes to about 3 hours. In some embodiments, the incubation is performed at an incubation period of about 2 hours.

In some embodiments, the concentration of drug in the drug-loaded platelets is from about 1 nM to about 1000 mM. In some embodiments, the concentration of drug in the drug-loaded platelets is from about 10 nM to about 10 mM. In some embodiments, the concentration of drug in the drug-loaded platelets is from about 100 nM to 1 mM.

In some embodiments, the method further comprises drying the drug-loaded platelets. In some embodiments, the drying step comprises freeze-drying the drug-loaded platelets. In some embodiments, the method further comprises rehydrating the drug-loaded platelets obtained from the drying step.

In some embodiments, drug-loaded platelets are prepared by using any one of the methods provided herein.

In some embodiments, rehydrated drug-loaded platelets are prepared by any one method comprising rehydrating the drug-loaded platelets provided herein.

The drug-loaded platelets may be then used, for example, for therapeutic applications as disclosed herein. As another example, the drug-loaded platelets may be employed in functional assays. In some embodiments, the drug-loaded platelets are cold stored, cryopreserved, or lyophilized (to produce thrombosomes) prior to use in therapy or in functional assays.

Any known technique for drying platelets can be used in accordance with the present disclosure, as long as the technique can achieve a final residual moisture content of less than 5%. Preferably, the technique achieves a final residual moisture content of less than 2%, such as 1%, 0.5%, or 0.1%. Non-limiting examples of suitable techniques are freeze-drying (lyophilization) and spray-drying. A suitable lyophilization method is presented in Table below. Additional exemplary lyophilization methods can be found in U.S. Pat. Nos. 7,811,558, 8,486,617, and U.S. Pat. No. 8,097,403. An exemplary spray-drying method includes: combining nitrogen, as a drying gas, with a loading buffer according to the present disclosure, then introducing the mixture into GEA Mobile Minor spray dryer from GEA Processing Engineering, Inc. (Columbia Md., USA), which has a Two-Fluid Nozzle configuration, spray drying the mixture at an inlet temperature in the range of 150° C. to 190° C., an outlet temperature in the range of 65° C. to 100° C., an atomic rate in the range of 0.5 to 2.0 bars, an atomic rate in the range of 5 to 13 kg/hr, a nitrogen use in the range of 60 to 100 kg/hr, and a run time of 10 to 35 minutes. The final step in spray drying is preferentially collecting the dried mixture. The dried composition in some embodiments is stable for at least six months at temperatures that range from −20° C. or lower to 90° C. or higher.

TABLE
Exemplary Lyophilization Protocol

| | Step | Temp. Set | Type | Duration | | Pressure Set |
|---|---|---|---|---|---|---|
| Freezing Step | F1 | −50° C. | Ramp | Var | | N/A |
| | F2 | −50° C. | Hold | 3 | Hrs | N/A |
| Vacuum Pulldown | F3 | −50º | Hold | Var | | N/A |
| Primary Dry | P1 | −40º | Hold | 1.5 | Hrs | 0 mT |
| | P2 | −35º | Ramp | 2 | Hrs | 0 mT |
| | P3 | −25º | Ramp | 2 | Hrs | 0 mT |
| | P4 | −17° C. | Ramp | 2 | Hrs | 0 mT |
| | P5 | 0° C. | Ramp | 1.5 | Hrs | 0 mT |
| | P6 | 27° C. | Ramp | 1.5 | Hrs | 0 mT |
| | P7 | 27° C. | Hold | 16 | Hrs | 0 mT |
| Secondary Dry | S1 | 27° C. | Hold | >8 | Hrs | 0 mT |

In some embodiments, the step of drying the drug-loaded platelets that are obtained as disclosed herein, such as the step of freeze-drying the drug-loaded platelets that are obtained as disclosed herein, comprises incubating the platelets with a lyophilizing agent (e.g., a non-reducing disaccharide. Accordingly, in some embodiments, the methods for preparing drug-loaded platelets further comprise incubating the drug-loaded platelets with a lyophilizing agent

In some embodiments the lyophilizing agent is a saccharide. In some embodiments the saccharide is a disaccharide, such as a non-reducing disaccharide).

In some embodiments, the platelets are incubated with a lyophilizing agent for a sufficient amount of time and at a suitable temperature to load the platelets with the lyophilizing agent. Non-limiting examples of suitable lyophilizing agents are saccharides, such as monosaccharides and disaccharides, including sucrose, maltose, trehalose, glucose (e.g., dextrose), mannose, and xylose. In some embodiments, non-limiting examples of lyophilizing agent include serum albumin, dextran, polyvinyl pyrrolidone (PVP), starch, and hydroxyethyl starch (HES). In some embodiments, exemplary lyophilizing agents can include a high molecular weight polymer, into the loading composition. By “high molecular weight” it is meant a polymer having an average molecular weight of about or above 70 kDa. Non-limiting examples are polymers of sucrose and epichlorohydrin. In some embodiments, the lyophilizing agent is polysucrose. Although any amount of high molecular weight polymer can be used as a lyophilizing agent, it is preferred that an amount be used that achieves a final concentration of about 3% to 10% (w/v), such as 3% to 7%, for example 6%.

In some embodiments, the process for preparing a composition includes adding an organic solvent, such as ethanol, to the loading solution. In such a loading solution, the solvent can range from 0.1% to 5.0% (v/v).

Within the process provided herein for making the compositions provided herein, addition of the lyophilizing agent can be the last step prior to drying. However, in some embodiments, the lyophilizing agent is added at the same time or before the drug, the cryoprotectant, or other components of the loading composition. In some embodiments, the lyophilizing agent is added to the loading solution, thoroughly mixed to form a drying solution, dispensed into a drying vessel (e.g., a glass or plastic serum vial, a lyophilization bag), and subjected to conditions that allow for drying of the solution to form a dried composition.

An exemplary saccharide for use in the compositions disclosed herein is trehalose. Regardless of the identity of the saccharide, it can be present in the composition in any suitable amount. For example, it can be present in an amount of 1 mM to 1 M. In some embodiments, it is present in an amount of from 10 mM 10 to 500 mM. In some embodiments, it is present in an amount of from 20 mM to 200 mM. In some embodiments, it is present in an amount from 40 mM to 100 mM. In various embodiments, the saccharide is present in different specific concentrations within the ranges recited above, and one of skill in the art can immediately understand the various concentrations without the need to specifically recite each herein. Where more than one saccharide is present in the composition, each saccharide can be present in an amount according to the ranges and particular concentrations recited above.

The step of incubating the platelets to load them with a cryoprotectant or as a lyophilizing agent includes incubating the platelets for a time suitable for loading, as long as the time, taken in conjunction with the temperature, is sufficient for the cryoprotectant or lyophilizing agent to come into contact with the platelets and, preferably, be incorporated, at least to some extent, into the platelets. In some embodiments, incubation is carried out for about 1 minute to about 180 minutes or longer.

The step of incubating the platelets to load them with a cryoprotectant or lyophilizing agent includes incubating the platelets and the cryoprotectant at a temperature that, when selected in conjunction with the amount of time allotted for loading, is suitable for loading. In general, the composition is incubated at a temperature above freezing for at least a sufficient time for the cryoprotectant or lyophilizing agent to come into contact with the platelets. In embodiments, incubation is conducted at 37° C. In certain embodiments, incubation is performed at 20° C. to 42° C. For example, in embodiments, incubation is performed at 35° C. to 40° C. (e.g., 37° C.) for 110 to 130 (e.g., 120) minutes.

In various embodiments, the bag is a gas-permeable bag configured to allow gases to pass through at least a portion or all portions of the bag during the processing. The gas-permeable bag can allow for the exchange of gas within the interior of the bag with atmospheric gas present in the surrounding environment. The gas-permeable bag can be permeable to gases, such as oxygen, nitrogen, water, air, hydrogen, and carbon dioxide, allowing gas exchange to occur in the compositions provided herein. In some embodiments, the gas-permeable bag allows for the removal of some of the carbon dioxide present within an interior of the bag by allowing the carbon dioxide to permeate through its wall. In some embodiments, the release of carbon dioxide from the bag can be advantageous to maintaining a desired pH level of the composition contained within the bag.

In some embodiments, the container of the process herein is a gas-permeable container that is closed or sealed. In some embodiments, the container is a container that is closed or sealed and a portion of which is gas-permeable. In some embodiments, the surface area of a gas-permeable portion of a closed or sealed container (e.g., bag) relative to the volume of the product being contained in the container (hereinafter referred to as the “SA/V ratio”) can be adjusted to improve pH maintenance of the compositions provided herein. For example, in some embodiments, the SA/V ratio of the container can be at least about 2.0 mL/cm² (e.g., at least about 2.1 mL/cm², at least about 2.2 mL/cm², at least about 2.3 mL/cm², at least about 2.4 mL/cm², at least about 2.5 mL/cm², at least about 2.6 mL/cm², at least about 2.7 mL/cm², at least about 2.8 mL/cm², at least about 2.9 mL/cm², at least about 3.0 mL/cm², at least about 3.1 mL/cm², at least about 3.2 mL/cm², at least about 3.3 mL/cm², at least about 3.4 mL/cm², at least about 3.5 mL/cm², at least about 3.6 mL/cm², at least about 3.7 mL/cm², at least about 3.8 mL/cm², at least about 3.9 mL/cm², at least about 4.0 mL/cm², at least about 4.1 mL/cm², at least about 4.2 mL/cm², at least about 4.3 mL/cm², at least about 4.4 mL/cm², at least about 4.5 mL/cm², at least about 4.6 mL/cm², at least about 4.7 mL/cm², at least about 4.8 mL/cm², at least about 4.9 mL/cm², or at least about 5.0 mL/cm². In some embodiments, the SA/V ratio of the container can be at most about 10.0 mL/cm² (e.g., at most about 9.9 mL/cm², at most about 9.8 mL/cm², at most about 9.7 mL/cm², at most about 9.6 mL/cm², at most about 9.5 mL/cm², at most about 9.4 mL/cm², at most about 9.3 mL/cm², at most about 9.2 mL/cm², at most about 9.1 mL/cm², at most about 9.0 mL/cm², at most about 8.9 mL/cm², at most about 8.8 mL/cm², at most about 8.7 mL/cm², at most about 8.6, mL/cm² at most about 8.5 mL/cm², at most about 8.4 mL/cm², at most about 8.3 mL/cm², at most about 8.2 mL/cm², at most about 8.1 mL/cm², at most about 8.0 mL/cm², at most about 7.9 mL/cm², at most about 7.8 mL/cm², at most about 7.7 mL/cm², at most about 7.6 mL/cm², at most about 7.5 mL/cm², at most about 7.4 mL/cm², at most about 7.3 mL/cm², at most about 7.2 mL/cm², at most about 7.1 mL/cm², at most about 6.9 mL/cm², at most about 6.8 mL/cm², at most about 6.7 mL/cm², at most about 6.6 mL/cm², at most about 6.5 mL/cm², at most about 6.4 mL/cm², at most about 6.3 mL/cm², at most about 6.2 mL/cm², at most about 6.1 mL/cm², at most about 6.0 mL/cm², at most about 5.9 mL/cm², at most about 5.8 mL/cm², at most about 5.7 mL/cm², at most about 5.6 mL/cm², at most about 5.5 mL/cm², at most about 5.4 mL/cm², at most about 5.3 mL/cm², at most about 5.2 mL/cm², at most about 5.1 mL/cm², at most about 5.0 mL/cm², at most about 4.9 mL/cm², at most about 4.8 mL/cm², at most about 4.7 mL/cm², at most about 4.6 mL/cm², at most about 4.5 mL/cm², at most about 4.4 mL/cm², at most about 4.3 mL/cm², at most about 4.2 mL/cm², at most about 4.1 mL/cm², or at most about 4.0 mL/cm². In some embodiments, the SA/V ratio of the container can range from about 2.0 to about 10.0 mL/cm² (e.g., from about 2.1 mL/cm² to about 9.9 mL/cm², from about 2.2 mL/cm² to about 9.8 mL/cm², from about 2.3 mL/cm² to about 9.7 mL/cm², from about 2.4 mL/cm² to about 9.6 mL/cm², from about 2.5 mL/cm² to about 9.5 mL/cm², from about 2.6 mL/cm² to about 9.4 mL/cm², from about 2.7 mL/cm² to about 9.3 mL/cm², from about 2.8 mL/cm² to about 9.2 mL/cm², from about 2.9 mL/cm² to about 9.1 mL/cm², from about 3.0 mL/cm² to about 9.0 mL/cm², from about 3.1 mL/cm² to about 8.9 mL/cm², from about 3.2 mL/cm² to about 8.8 mL/cm², from about 3.3 mL/cm² to about 8.7 mL/cm², from about 3.4 mL/cm² to about 8.6 mL/cm², from about 3.5 mL/cm² to about 8.5 mL/cm², from about 3.6 mL/cm² to about 8.4 mL/cm², from about 3.7 mL/cm² to about 8.3 mL/cm², from about 3.8 mL/cm² to about 8.2 mL/cm², from about 3.9 mL/cm² to about 8.1 mL/cm², from about 4.0 mL/cm² to about 8.0 mL/cm², from about 4.1 mL/cm² to about 7.9 mL/cm², from about 4.2 mL/cm² to about 7.8 mL/cm², from about 4.3 mL/cm² to about 7.7 mL/cm², from about 4.4 mL/cm² to about 7.6 mL/cm², from about 4.5 mL/cm² to about 7.5 mL/cm², from about 4.6 mL/cm² to about 7.4 mL/cm², from about 4.7 mL/cm² to about 7.3 mL/cm², from about 4.8 mL/cm² to about 7.2 mL/cm², from about 4.9 mL/cm² to about 7.1 mL/cm², from about 5.0 mL/cm² to about 6.9 mL/cm², from about 5.1 mL/cm² to about 6.8 mL/cm², from about 5.2 mL/cm² to about 6.7 mL/cm², from about 5.3 mL/cm² to about 6.6 mL/cm², from about 5.4 mL/cm² to about 6.5 mL/cm², from about 5.5 mL/cm² to about 6.4 mL/cm², from about 5.6 mL/cm² to about 6.3 mL/cm², from about 5.7 mL/cm² to about 6.2 mL/cm², or from about 5.8 mL/cm² to about 6.1 mL/cm².

Gas-permeable closed containers (e.g., bags) or portions thereof can be made of one or more various gas-permeable materials. In some embodiments, the gas-permeable bag can be made of one or more polymers including fluoropolymers (such as polytetrafluoroethylene (PTFE) and perfluoroalkoxy (PFA) polymers), polyolefins (such as low-density polyethylene (LDPE), high-density polyethylene (HDPE)), fluorinated ethylene propylene (FEP), polystyrene, polyvinylchloride (PVC), silicone, and any combinations thereof.

In some embodiments the lyophilizing agent as disclosed herein may be a high molecular weight polymer. By “high molecular weight” it is meant a polymer having an average molecular weight of about or above 70 kDa and up to 1,000,000 kDa. Non-limiting examples are polymers of sucrose and epichlorohydrin (polysucrose). Although any amount of high molecular weight polymer can be used, it is preferred that an amount be used that achieves a final concentration of about 3% to 10% (w/v), such as 3% to 7%, for example 6%. Other non-limiting examples of lyoprotectants are serum albumin, dextran, polyvinyl pyrrolidone (PVP), starch, and hydroxyethyl starch (HES).

In some embodiments, the loading buffer comprises an organic solvent, such as an alcohol (e.g., ethanol). In such a loading buffer, the amount of solvent can range from 0.1% to 5.0% (v/v).

In some embodiments the drug-loaded platelets prepared as disclosed herein have a storage stability that is at least about equal to that of the platelets prior to the loading of the drug.

The loading buffer may be any buffer that is non-toxic to the platelets and provides adequate buffering capacity to the solution at the temperatures at which the solution will be exposed during the process provided herein. Thus, the buffer may comprise any of the known biologically compatible buffers available commercially, such as phosphate buffers, such as phosphate buffered saline (PBS), bicarbonate/carbonic acid, such as sodium-bicarbonate buffer, N-2-hydroxyethylpiperazine-N′-2-ethanesulfonic acid (HEPES), and tris-based buffers, such as tris-buffered saline (TBS). Likewise, it may comprise one or more of the following buffers: propane-1,2,3-tricarboxylic (tricarballylic); benzenepentacarboxylic; maleic; 2,2-dimethylsuccinic; EDTA; 3,3-dimethylglutaric; bis(2-hydroxyethyl)imino-tris(hydroxymethyl)-methane (BIS-TRIS); benzenehexacarboxylic (mellitic); N-(2-acetamido)imino-diacetic acid (ADA); butane-1,2,3,4-tetracarboxylic; pyrophosphoric; 1,1-cyclopentanediacetic (3,3 tetramethylene-glutaric acid); piperazine-1,4-bis-(2-ethanesulfonic acid) (PIPES); N-(2-acetamido)-2-amnoethanesulfonic acid (ACES); 1,1-cyclohexanediacetic; 3,6-endomethylene-1,2,3,6-tetrahydrophthalic acid (EMTA; ENDCA); imidazole; 2-(aminoethyl)trimethylammonium chloride (CHOLAMINE); N,N-bis(2-hydroxyethyl)-2-aminoethanesulfonic acid (BES); 2-methylpropane-1,2,3-triscarboxylic (beta-methyltricarballylic); 2-(N-morpholino)propane-sulfonic acid (MOPS); phosphoric; and N-tris(hydroxymethyl)methyl-2-amminoethane sulfonic acid (TES).

Flow cytometry is used to obtain a relative quantification of loading efficiency by measuring the mean fluorescence intensity of the drug in the drug-loaded platelets. Platelets are evaluated for functionality by ADP and/or TRAP stimulation post-loading.

In some embodiments the drug-loaded platelets are lyophilized. In some embodiments the drug-loaded platelets are cryopreserved.

In some embodiments the drug-loaded platelets retain the loaded drug upon rehydration and release the drug upon stimulation by endogenous platelet activators.

In some embodiments the dried platelets (such as freeze-dried platelets) retain the loaded drug upon rehydration and release the drug upon stimulation by endogenous platelet activators. In some embodiments at least about 10%, such as at least about 20%, such as at least about 30% of the drug is retained. In some embodiments from about 10% to about 20%, such as from about 20% to about 30% of the drug is retained.

An example of a drug that may be loaded in a platelet is doxorubicin. Another example is of a drug that may be loaded in a platelet is olaparib. Another example of a drug that may loaded in a platelet is paclitaxel.

Various agents and/or procedures may be used to load the platelets with a drug. In some embodiments, the platelets are loaded with a liposomal formulation of the drug. In some embodiments, the drug is not comprised in a liposomal formulation. In some embodiments, the platelets are loaded with a drug previously incubated with a cell penetrating peptide. In some embodiments, the platelets are loaded with a drug previously incubated with a cationic lipid such as lipofectamine. In some embodiments, the platelets are loaded with the drug in the presence of a detergent. For example, the detergent may be saponin.

In some embodiments, the platelets are loaded by a process comprising endocytosis.

In some embodiments, the platelets are loaded by a process comprising electroporation.

In some embodiments, the platelets are loaded by a process comprising transduction.

In some embodiments, the platelets are loaded by a process comprising sonoporation.

In some embodiments, the platelets are loaded by a process comprising osmotic hypertonic/hypotonic loading/hypotonic shock. Hypotonic shock uses a solution with lower osmotic pressure to induce cell swelling leading to membrane permeability. Hypertonic shock may increase platelet loading of cryoprotectants or lyoprotectants (e.g., trehalose) (Zhou X., et. al., Loading Trehalose into Red Blood Cells by Improved Hypotonic Method, Cell Preservation Technology, 6(2), https://doi.org/10.1089/cpt.2008.0001 (2008), which is herein incorporated by reference). Additionally and alternatively, hypotonic shock may allow the uptake and internalization of large and/or charged molecules through passive means, such as, endocytosis, micropinocytosis, and/or diffusion.

In some embodiments, the solutes in the hypertonic solution can be, in a non-limiting way, salts, low-molecular weight sugars (e.g., monosaccharides, disaccharides), or low molecular weight inert hydrophilic polymers.

In some embodiments, the platelets are loaded by a process comprising the use of Transfection Reagents (also described in WO2014118817A2, incorporated by reference herein in its entirety).

### Exemplary Embodiments

Provided in this Exemplary Embodiments section are non-limiting exemplary aspects and embodiments provided herein and further discussed throughout this specification. For the sake of brevity and convenience, all of the aspects and embodiments disclosed herein, and all of the possible combinations of the disclosed aspects and embodiments are not listed in this section. Additional embodiments and aspects are provided in other sections herein. Furthermore, it will be understood that embodiments are provided that are specific embodiments for many aspects and that can be combined with any other embodiment, for example as discussed in this entire disclosure. It is intended in view of the full disclosure herein, that any individual embodiment recited below or in this full disclosure can be combined with any aspect recited below or in this full disclosure where it is an additional element that can be added to an aspect or because it is a narrower element for an element already present in an aspect. Such combinations are sometimes provided as non-limiting exemplary combinations and/or are discussed more specifically in other sections of this detailed description.

1. A method of preparing drug-loaded platelets, comprising:
treating platelets with a drug and with a loading buffer comprising a salt, a base, a loading agent, and optionally at least one organic solvent,
to form the drug-loaded platelets.

2. A method of preparing drug-loaded platelets, comprising:
a) providing platelets;
   and
b) treating the platelets with a drug and with a loading buffer comprising a salt, a base, a loading agent, and optionally at least one organic solvent
to form the drug-loaded platelets.

3. The method of any one of the preceding embodiments, wherein the platelets are treated with the drug and with the buffer sequentially, in either order.

4. A method of preparing drug-loaded platelets, comprising:
(1) treating platelets with a drug to form a first composition; and
(2) treating the first composition with a buffer comprising a salt, a base, a loading agent, and optionally at least one organic solvent, to form the drug-loaded platelets.

5. A method of preparing drug-loaded platelets, comprising:
(1) treating the platelets with a buffer comprising a salt, a base, a loading agent, and optionally at least one organic solvent to form a first composition; and
(2) treating the first composition with a drug, to form the drug-loaded platelets.

6. The method of embodiment 1 or 2, wherein the platelets are treated with the drug and with the buffer concurrently.

7. A method of preparing drug-loaded platelets, comprising:
treating the platelets with a drug in the presence of a buffer comprising a salt, a base, a loading agent, and optionally at least one organic solvent to form the drug-loaded platelets.

8. The method of any one of the preceding embodiments, wherein the platelets are pooled from a plurality of donors prior to a treating step.

9. A method of preparing drug-loaded platelets comprising
A) pooling platelets from a plurality of donors; and
B) treating the platelets from step (A) with a drug and with a loading buffer comprising a salt, a base, a loading agent, and optionally at least one organic solvent, to form the drug-loaded platelets.

10. A method of preparing drug-loaded platelets comprising
A) pooling platelets from a plurality of donors; and
B)
   (1) treating the platelets from step (A) with a drug to form a first composition; and
   (2) treating the first composition with a buffer comprising a salt, a base, a loading agent, and optionally at least one organic solvent, to form the drug-loaded platelets.

11. A method of preparing drug-loaded platelets comprising
A) pooling platelets from a plurality of donors; and
B)
   treating the platelets from step (A) with a buffer comprising a salt, a base, a loading agent, and optionally at least one organic solvent, to form a first composition; and
   (C) treating the first composition with a drug to form the drug-loaded platelets.

12. A method of preparing drug-loaded platelets comprising
A) pooling platelets from a plurality of donors; and
B) treating the platelets with a drug in the presence of a buffer comprising a salt, a base, a loading agent, and optionally at least one organic solvent, to form the drug-loaded platelets.

13. The method of any one of the preceding embodiments, wherein the loading agent is a monosaccharide or a disaccharide.

14. The method of any one of the preceding embodiments, wherein the loading agent is sucrose, maltose, trehalose, glucose, mannose, or xylose.

15. The method of any one of the preceding embodiments, wherein the platelets are isolated prior to a treating step.

16. The method of any one of the preceding embodiments, wherein the platelets are loaded with the drug in a period of time of 5 minutes to 48 hours.

17. The method of any one of the preceding embodiments, wherein the concentration of drug in the drug-loaded platelets is from about 1 nM to about 100 mM.

18. The method of any one of the preceding embodiments, wherein the one or more organic solvents selected from the group consisting of ethanol, acetic acid, acetone, acetonitrile, dimethylformamide, dimethyl sulfoxide, dioxane, methanol, n-propanol, isopropanol, tetrahydrofuran (THF), N-methyl pyrrolidone, dimethylacetamide (DMAC), or combinations thereof.

19. The method of any one of the preceding embodiments, further comprising cold storing, cryopreserving, freeze-drying, thawing, rehydrating, and combinations thereof the drug-loaded platelets.

20. The method of embodiment 19, wherein the drying step comprises freeze-drying the drug-loaded platelets.

21. The method of embodiment 19 or 20, further comprising rehydrating the drug-loaded platelets obtained from the drying step.

22. Drug-loaded platelets prepared by the method of any one of the preceding embodiments.

23. Rehydrated drug-loaded platelets prepared by a method comprising rehydrating the drug-loaded platelets of c embodiment 22.

24. The method of any one of the preceding embodiments, wherein the drug is modified with an imaging agent.

25. The method of embodiment 24, wherein the drug is modified with the imaging agent prior to treating platelets with the drug.

26. The method of any one of the preceding embodiments, wherein the platelets are further treated with an imaging agent, wherein the drug-loaded platelets are loaded with the imaging agent.

27. The method of any one of the preceding embodiments, wherein the method does not comprise treating the platelets with an organic solvent.

28. The method of any one of embodiments 4, 5, 10 or 11, wherein the method does not comprise treating the first composition with an organic solvent.

29. The method of any one of the preceding embodiments, wherein the method comprises treating the platelets with Prostaglandin E1 (PGE1) or Prostacyclin.

30. The method of any one of embodiments 1 to 28, wherein the method does not comprise treating the platelets with Prostaglandin E1 (PGE1) or Prostacyclin.

31. The method of any one of the preceding embodiments, wherein the method comprises treating the platelets with a chelating agent such as EGTA.

32. The method of any one of embodiments 1 to 30, wherein the method does not comprises treating the platelets with a chelating agent such as EGTA.

33. The method of any one of embodiments 1 to 29, wherein the method comprises treating the first composition with Prostaglandin E1 (PGE1) or Prostacyclin.

34. The method of any one of embodiments 1 to 28 or 30, wherein the method does not comprise treating the first composition with Prostaglandin E1 (PGE1) or Prostacyclin.

35. The method of any one of embodiments 1 to 31, 33 or 34, wherein the method comprises treating the first composition with a chelating agent such as EGTA.

36. The method of any one of embodiments 1 to 30 or 32 to 34, wherein the method does not comprises treating the first composition with a chelating agent such as EGTA.

37. The method of any one of the preceding embodiments, wherein the method further comprises treating the drug-loaded platelets with an anti-aggregation agent.

38. The method of embodiment 37, wherein the anti-aggregation agent is a GPIIb/IIIa inhibitor.

39. The method of embodiment 38, wherein the GPIIb/IIIa inhibitor is GR144053.

40. The method of embodiment 39, wherein GR144053 is present in a concentration of at least 1.2 μM.

41. The method of any one of embodiments 38 to 41, wherein the platelets are treated with the anti-aggregation agent before being treated with the drug.

42. The method of any one of embodiments 38 to 41, wherein the platelets are treated with the anti-aggregation agent concurrently with the drug.

43. The method of any one of the preceding claims, wherein the drug is a small molecule, a protein, an oligopeptide, an aptamer, and combinations thereof.

44. The method of any one of the preceding embodiments, wherein the drug is a drug for the treatment of cancer.

45. The method of embodiment 44, wherein the cancer comprises hemangiosarcoma.

46. The method of embodiments 44 to 45, wherein the drug for the treatment of cancer is doxorubicin.

47. The method of embodiments 45 to 46, wherein the drug for the treatment of hemangiosarcoma is doxorubicin.

48. The method of embodiment 44, wherein the drug for the treatment of cancer is paclitaxel.

49. The method of embodiment 44, wherein the drug for the treatment of cancer is a PARP inhibitor.

50. The method of embodiment 49, wherein the PARP inhibitor is olaparib.

51. A method of preparing RNA agent-loaded platelets, comprising:
treating platelets with a RNA agent a cationic transfection reagent; and a loading buffer comprising a salt, a base, a loading agent, and optionally at least one organic solvent, to form the RNA agent-loaded platelets.

52. A method of preparing RNA agent-loaded platelets, comprising:
a) providing platelets; and
b) treating the platelets with a RNA agent; a cationic transfection reagent; and a loading buffer comprising a salt, a base, a loading agent, and optionally at least one organic solvent to form the RNA agent-loaded platelets.

53. The method of any one of embodiments 51 or 52, wherein the platelets are treated with the RNA agent and with the loading buffer sequentially, in either order.

54. The method of any one of embodiments 51 to 53, wherein the platelets are treated with the RNA agent and with the cationic transfection reagent sequentially, in either order.

55. A method of preparing RNA agent-loaded platelets, comprising:
(1) treating platelets with an RNA agent to form a first composition; and
(2) treating the first composition with a loading buffer comprising a salt, a base, a loading agent, and optionally at least one organic solvent, to form the RNA agent-loaded platelets.

56. The method of embodiment 55, wherein the first composition is treated with a cationic transfection reagent.

57. The method of embodiment 56, wherein the first composition treated with the cationic transfection reagent is treated with a loading buffer comprising a salt, a base, a loading agent, and optionally at least one organic solvent, to form the RNA agent-loaded platelets.

58. A method of preparing RNA agent-loaded platelets, comprising:
(1) treating the platelets with a loading buffer comprising a salt, a base, a loading agent, and optionally at least one organic solvent to form a first composition; and
(2) treating the first composition with a RNA agent, to form the RNA agent-loaded platelets.

59. The method of embodiment 58, wherein the first composition is treated with a cationic transfection reagent.

60. The method of embodiment 59, wherein the first composition treated with the cationic transfection reagent is treated with a RNA agent, to form the RNA agent-loaded platelets.

61. The method of embodiment 51 or 52, wherein the platelets are treated with the RNA agent and with the loading buffer concurrently.

62. The method of embodiment 51 or 52, wherein the platelets are treated with the RNA agent and with the cationic transfection reagent concurrently.

63. A method of preparing RNA agent-loaded platelets, comprising: treating the platelets with a RNA agent in the presence of a cationic transfection reagent and a loading buffer comprising a salt, a base, a loading agent, and optionally at least one organic solvent to form the RNA agent-loaded platelets.

64. The method of any one of embodiments 51 to 63, wherein the platelets are pooled from a plurality of donors.

65. A method of preparing RNA agent-loaded platelets comprising
A) pooling platelets from a plurality of donors; and
B) treating the platelets from step (A) with a RNA agent; a cationic transfection reagent; and with a loading buffer comprising a salt, a base, a loading agent, and optionally at least one organic solvent, to form the RNA agent-loaded platelets.

66. A method of preparing RNA agent-loaded platelets comprising
A) pooling platelets from a plurality of donors; and
B)
   (1) treating the platelets from step (A) with a RNA agent to form a first composition; and
   (2) treating the first composition with a loading buffer comprising a salt, a base, a loading agent, and optionally at least one organic solvent, to form the RNA agent-loaded platelets.

67. The method of embodiment 66, wherein the first composition is treated with a cationic transfection reagent.

68. The method of embodiment 67, wherein the first composition treated with the cationic transfection agent is treated with a loading buffer comprising a salt, a base, a loading agent and optionally at least one organic solvent, to form the RNA agent-loaded platelets.

69. A method of preparing RNA agent-loaded platelets comprising
A) pooling platelets from a plurality of donors; and
B)
   (1) treating the platelets from step (A) with a loading buffer comprising a salt, a base, a loading agent, and optionally at least one organic solvent, to form a first composition; and
   (2) treating the first composition with a RNA agent to form the RNA agent-loaded platelets.

70. The method of embodiment 69, wherein the first composition is treated with a cationic transfection reagent.

71. The method of embodiment 70, wherein the first composition treated with the cationic transfection agent is treated with a loading buffer comprising a salt, a base, a loading agent, and optionally at least one organic solvent, to form the RNA agent-loaded platelets.

72. A method of preparing RNA agent-loaded platelets comprising
A) pooling platelets from a plurality of donors; and
B) treating the platelets with a RNA agent in the presence of a cationic transfection reagent and a loading buffer comprising a salt, a base, a loading agent, and optionally at least one organic solvent, to form the RNA agent-loaded platelets.

73. The method of any one of embodiments 51 to 72, wherein the loading buffer comprises optionally at least one organic solvent.

74. The method of any one of embodiments 51 to 73, wherein the loading agent is a monosaccharide or a disaccharide.

75. The method of any one of embodiments 51 to 74, wherein the loading agent is sucrose, maltose, dextrose, trehalose, glucose, mannose, or xylose.

76. The method of any one of embodiments 51 to 75, wherein the platelets are isolated prior to a treating step.

77. The method of any one of embodiments 51 to 76, wherein the platelets are selected from the group consisting of fresh platelets, stored platelet, and any combination thereof.

78. The method of any one of embodiments 51 to 77, wherein the cationic transfection reagent is a cationic lipid transfection reagent.

79. The method of any one of embodiments 51 to 78, wherein the RNA agent comprises siRNA.

80. The method of any one of embodiments 51 to 79, wherein the RNA agent comprises miRNA.

81. The method of any one of embodiments 51 to 80, wherein the platelets are loaded with the RNA agent in a period of time of 1 minute to 48 hours.

82. The method of any one of embodiments 51 to 81, wherein the concentration of RNA agent in the RNA agent-loaded platelets is from about 0.1 nM to about 10 μM.

83. The method of any one of embodiments 51 to 82, wherein the concentration of RNA agent in the RNA agent-loaded platelets is about 100 nM.

84. The method of any one of embodiments 51 to 83, wherein the one or more organic solvents selected from the group consisting of ethanol, acetic acid, acetone, acetonitrile, dimethylformamide, dimethyl sulfoxide, dioxane, methanol, n-propanol, isopropanol, tetrahydrofuran (THF), N-methyl pyrrolidone, dimethylacetamide (DMAC), or combinations thereof.

85. The method of any one of embodiments 51 to 84, further comprising cold storing, cryopreserving, freeze-drying, thawing, rehydrating, and combinations thereof the RNA agent-loaded platelets.

86. The method of embodiment 85, wherein the drying step comprises freeze-drying the RNA agent-loaded platelets.

87. The method of embodiment 85 or 86, further comprising rehydrating the RNA agent-loaded platelets obtained from the drying step.

88. RNA agent-loaded platelets prepared by the method of any one of embodiments 51 to 87.

89. Rehydrated RNA agent-loaded platelets prepared by a method comprising rehydrating the RNA agent-loaded platelets of embodiment 88.

90. The method of any one of embodiments 51 to 89, wherein the method does not comprise treating the platelets with an organic solvent.

91. The method of any one of embodiments 55 to 60 or 66 to 71, wherein the method does not comprise treating the first composition with an organic solvent.

92. The method of any one of embodiments 51 to 92, wherein the method comprises treating the platelets with Prostaglandin E1 (PGE1) or Prostacyclin.

93. The method of any one of embodiments 51 to 91, wherein the method does not comprise treating the platelets with Prostaglandin E1 (PGE1) or Prostacyclin.

94. The method of any one of embodiments 51 to 92, wherein the method comprises treating the first composition with Prostaglandin E1 (PGE1) or Prostacyclin.

95. The method of any one of embodiments 51 to 91 or 93, wherein the method does not comprise treating the first composition with Prostaglandin E1 (PGE1) or Prostacyclin.

96. A method of preparing drug-loaded platelets, comprising:
treating platelets with a drug and with a loading buffer comprising a salt, a base, a loading agent, and optionally at least one organic solvent, to form the drug-loaded platelets.

97. A method of preparing drug-loaded platelets, comprising:
a) providing platelets;
   and
b) treating the platelets with a drug and with a loading buffer comprising a salt, a base, a loading agent, and optionally at least one organic solvent to form the drug-loaded platelets.

98. The method of any one of the preceding embodiments, wherein the platelets are treated with the drug and with the buffer sequentially, in either order.

99. A method of preparing drug-loaded platelets, comprising:
(1) treating platelets with a drug to form a first composition; and
(2) treating the first composition with a buffer comprising a salt, a base, a loading agent, and optionally at least one organic solvent, to form the drug-loaded platelets.

100. A method of preparing drug-loaded platelets, comprising:
(1) treating the platelets with a buffer comprising a salt, a base, a loading agent, and optionally at least one organic solvent to form a first composition; and
(2) treating the first composition with a drug, to form the drug-loaded platelets.

101. The method of embodiments 96 or 97, wherein the platelets are treated with the drug and with the buffer concurrently.

102. A method of preparing drug-loaded platelets, comprising:
treating the platelets with a drug in the presence of a buffer comprising a salt, a base, a loading agent, and optionally at least one organic solvent to form the drug-loaded platelets.

103. The method of any one of embodiments 96 to 102, wherein the platelets are pooled from a plurality of donors prior to a treating step.

104. A method of preparing drug-loaded platelets comprising
A) pooling platelets from a plurality of donors; and
B) treating the platelets from step (A) with a drug and with a loading buffer comprising a salt, a base, a loading agent, and optionally at least one organic solvent, to form the drug-loaded platelets.

105. A method of preparing drug-loaded platelets comprising
A) pooling platelets from a plurality of donors; and
B)
   1. treating the platelets from step (A) with a drug to form a first composition; and
   2. treating the first composition with a buffer comprising a salt, a base, a loading agent, and optionally at least one organic solvent, to form the drug-loaded platelets.

106. A method of preparing drug-loaded platelets comprising
A) pooling platelets from a plurality of donors; and
B)
   1. treating the platelets from step (A) with a buffer comprising a salt, a base, a loading agent, and optionally at least one organic solvent, to form a first composition; and
   2. treating the first composition with a drug to form the drug-loaded platelets.

107. A method of preparing drug-loaded platelets comprising
A) pooling platelets from a plurality of donors; and
B) treating the platelets with a drug in the presence of a buffer comprising a salt, a base, a loading agent, and optionally at least one organic solvent, to form the drug-loaded platelets.

108. The method of any one of embodiments 96 to 107, wherein the loading agent is a monosaccharide or a disaccharide.

109. The method of any one of embodiments 96 to 108, wherein the loading agent is sucrose, maltose, trehalose, glucose, mannose, or xylose.

110. The method of any one of embodiments 96 to 109, wherein the platelets are isolated prior to a treating step.

111. The method of any one of embodiments 96 to 110, wherein the platelets are loaded with the drug in a period of time of 5 minutes to 48 hours.

112. The method of any one of embodiments 96 to 111, wherein the concentration of drug in the drug-loaded platelets is from about 1 nM to about 100 mM.

113. The method of any one of embodiments 96 to 112, wherein the one or more organic solvents selected from the group consisting of ethanol, acetic acid, acetone, acetonitrile, dimethylformamide, dimethyl sulfoxide, dioxane, methanol, n-propanol, isopropanol, tetrahydrofuran (THF), N-methyl pyrrolidone, dimethylacetamide (DMAC), or combinations thereof.

114. The method of any one of embodiments 96 to 113, further comprising cold storing, cryopreserving, freeze-drying, thawing, rehydrating, and combinations thereof the drug-loaded platelets.

115. The method of embodiment 114, wherein the drying step comprises freeze-drying the drug-loaded platelets.

116. The method of embodiment 114 or 115, further comprising rehydrating the drug-loaded platelets obtained from the drying step.

117. Drug-loaded platelets prepared by the method of any one of embodiments 96 to 116.

118. Rehydrated drug-loaded platelets prepared by a method comprising rehydrating the drug-loaded platelets of embodiment 117.

119. The method of any one of embodiments 96 to 118, wherein the drug is modified with an imaging agent.

120. The method of embodiment 24, wherein the drug is modified with the imaging agent prior to treating platelets with the drug.

121. The method of any one of embodiments 96 to 120, wherein the platelets are further treated with an imaging agent, wherein the drug-loaded platelets are loaded with the imaging agent.

122. The method of any one of embodiments 96 to 121, wherein the method does not comprise treating the platelets with an organic solvent.

123. The method of any one of embodiments 99, 100, 105 or 106, wherein the method does not comprise treating the first composition with an organic solvent.

124. The method of any one of embodiments 96 to 123, wherein the method comprises treating the platelets with Prostaglandin E1 (PGE1) or Prostacyclin.

125. The method of any one of embodiments 96 to 123, wherein the method does not comprise treating the platelets with Prostaglandin E1 (PGE1) or Prostacyclin.

126. The method of any one of embodiments 96 to 125, wherein the method comprises treating the platelets with a chelating agent such as EGTA.

127. The method of any one of embodiments 96 to 125, wherein the method does not comprises treating the platelets with a chelating agent such as EGTA.

128. The method of any one of embodiments 96 to 124, wherein the method comprises treating the first composition with Prostaglandin E1 (PGE1) or Prostacyclin.

129. The method of any one of embodiments 96 to 123 or 125, wherein the method does not comprise treating the first composition with Prostaglandin E1 (PGE1) or Prostacyclin.

130. The method of any one of embodiments 96 to 126, 128 or 129, wherein the method comprises treating the first composition with a chelating agent such as EGTA.

131. The method of any one of embodiments 96 to 125 or 127 to 129, wherein the method does not comprise treating the first composition with a chelating agent such as EGTA.

132. The method of any one of embodiments 96 to 131, wherein the drug is a small molecule, a protein, an oligopeptide, an aptamer, or combinations thereof.

133. A method of preparing mRNA agent-loaded platelets, comprising:
treating platelets with a mRNA agent
a cationic transfection reagent;
and a loading buffer comprising a salt, a base, a loading agent, and
optionally at least one organic solvent,
to form the mRNA agent-loaded platelets.

134. A method of preparing mRNA agent-loaded platelets, comprising:
a) providing platelets;
and
b) treating the platelets with a mRNA agent; a cationic transfection reagent; and a loading buffer comprising a salt, a base, a loading agent, and optionally at least one organic solvent to form the mRNA agent-loaded platelets.

135. The method of any one of embodiments 133 or 134, wherein the platelets are treated with the mRNA agent and with the loading buffer sequentially, in either order.

136. The method of any one of the preceding embodiments, wherein the platelets are treated with the mRNA agent and with the cationic transfection reagent sequentially, in either order.

137. A method of preparing mRNA agent-loaded platelets, comprising:
(1) treating platelets with an mRNA agent to form a first composition; and
(2) treating the first composition with a loading buffer comprising a salt, a base, a loading agent, and optionally at least one organic solvent, to form the mRNA agent-loaded platelets.

138. The method of embodiment 137, wherein the first composition is treated with a cationic transfection reagent.

139. The method of embodiment 138, wherein the first composition treated with the cationic transfection reagent is treated with a loading buffer comprising a salt, a base, a loading agent, and optionally at least one organic solvent, to form the mRNA agent-loaded platelets.

140. A method of preparing mRNA agent-loaded platelets, comprising:
(1) treating the platelets with a loading buffer comprising a salt, a base, a loading agent, and optionally at least one organic solvent to form a first composition; and
(2) treating the first composition with a mRNA agent, to form the mRNA agent-loaded platelets.

141. The method of embodiment 140, wherein the first composition is treated with a cationic transfection reagent.

142. The method of embodiment 141, wherein the first composition treated with the cationic transfection reagent is treated with a mRNA agent, to form the mRNA agent-loaded platelets.

143. The method of embodiment 133 or 134, wherein the platelets are treated with the mRNA agent and with the loading buffer concurrently.

144. The method of embodiment 133 or 134, wherein the platelets are treated with the mRNA agent and with the cationic transfection reagent concurrently.

145. A method of preparing mRNA agent-loaded platelets, comprising:
treating the platelets with a mRNA agent in the presence of a cationic transfection reagent and a loading buffer comprising a salt, a base, a loading agent, and optionally at least one organic solvent to form the mRNA agent-loaded platelets.

146. The method of any one of embodiments 133 to 145, wherein the platelets are pooled from a plurality of donors.

147. A method of preparing mRNA agent-loaded platelets comprising:
A) pooling platelets from a plurality of donors; and
B) treating the platelets from step (A) with a mRNA agent; a cationic transfection reagent; and with a loading buffer comprising a salt, a base, a loading agent, and optionally at least one organic solvent, to form the mRNA agent-loaded platelets.

148. A method of preparing mRNA agent-loaded platelets comprising
A) pooling platelets from a plurality of donors; and
B)
   (1) treating the platelets from step (A) with a mRNA agent to form a first composition; and
   (2) treating the first composition with a loading buffer comprising a salt, a base, a loading agent, and optionally at least one organic solvent, to form the mRNA agent-loaded platelets.

149. The method of embodiment 148, wherein the first composition is treated with a cationic transfection reagent.

150. The method of embodiment 149, wherein the first composition treated with the cationic transfection agent is treated with a loading buffer comprising a salt, a base, a loading agent and optionally at least one organic solvent, to form the mRNA agent-loaded platelets.

151. A method of preparing mRNA agent-loaded platelets comprising
A) pooling platelets from a plurality of donors; and
B)
   (1) treating the platelets from step (A) with a loading buffer comprising a salt, a base, a loading agent, and optionally at least one organic solvent, to form a first composition; and
   (2) treating the first composition with a mRNA agent to form the mRNA agent-loaded platelets.

152. The method of embodiment 151, wherein the first composition is treated with a cationic transfection reagent.

153. The method of embodiment 152, wherein the first composition treated with the cationic transfection agent is treated with a loading buffer comprising a salt, a base, a loading agent, and optionally at least one organic solvent, to form the mRNA agent-loaded platelets.

154. A method of preparing mRNA agent-loaded platelets comprising
A) pooling platelets from a plurality of donors; and
B) treating the platelets with a mRNA agent in the presence of a cationic transfection reagent and a loading buffer comprising a salt, a base, a loading agent, and optionally at least one organic solvent, to form the mRNA agent-loaded platelets.

155. The method of any one of embodiments 133 to 154, wherein the loading buffer comprises optionally at least one organic solvent.

156. The method of any one of embodiments 133 to 155, wherein the loading agent is a monosaccharide or a disaccharide.

157. The method of any one of embodiments 133 to 156, wherein the loading agent is sucrose, maltose, dextrose, trehalose, glucose, mannose, or xylose.

158. The method of any one of embodiments 133 to 157, wherein the platelets are isolated prior to a treating step.

159. The method of any one of embodiments 133 to 158, wherein the platelets are selected from the group consisting of fresh platelets, stored platelet, and any combination thereof.

160. The method of any one of embodiments 133 to 159, wherein the cationic transfection reagent is a cationic lipid transfection reagent.

161. The method of any one of embodiments 133 to 160, wherein the mRNA agent comprises mRNA.

162. The method of any one of embodiments 133 to 161, wherein the platelets are loaded with the mRNA agent in a period of time of 1 minute to 48 hours.

163. The method of any one of embodiments 133 to 162, wherein the concentration of mRNA agent in the mRNA agent-loaded platelets is from about 0.1 nM to about 10 μM.

164. The method of any one of embodiments 133 to 163, wherein the concentration of mRNA agent in the mRNA agent-loaded platelets is about 100 nM.

165. The method of any one of embodiments 133 to 164, wherein the one or more organic solvents selected from the group consisting of ethanol, acetic acid, acetone, acetonitrile, dimethylformamide, dimethyl sulfoxide, dioxane, methanol, n-propanol, isopropanol, tetrahydrofuran (THF), N-methyl pyrrolidone, dimethylacetamide (DMAC), or combinations thereof.

166. The method of any one of embodiments 133 to 165, further comprising cold storing, cryopreserving, freeze-drying, thawing, rehydrating, and combinations thereof the mRNA agent-loaded platelets.

167. The method of embodiment 166, wherein the drying step comprises freeze-drying the mRNA agent-loaded platelets.

168. The method of embodiment 166 or 167, further comprising rehydrating the mRNA agent-loaded platelets obtained from the drying step.

169. mRNA agent-loaded platelets prepared by the method of any one of embodiments 133 to 168.

170. Rehydrated mRNA agent-loaded platelets prepared by a method comprising rehydrating the mRNA agent-loaded platelets of embodiment 169.

171. The method of any one of embodiments 133 to 170, wherein the method does not comprise treating the platelets with an organic solvent.

172. The method of any one of embodiments 137 to 142 or 148 to 153, wherein the method does not comprise treating the first composition with an organic solvent.

### EXAMPLES

Exemplary protocols that employ the foregoing agents or procedures are described below:

A liposome is a vesicle made of phospholipid bilayer. This vesicle can be designed to encapsulate drug of interest, which is delivered inside a cell following the fusion of vesicle and cell membrane.

Liposome encapsulated Doxorubicin (chemotherapy drug) is prepared through rehydration of lyophilized lipids (Sigma-Aldrich, L4395-1VL) with drug in PBS followed by 30 seconds of agitation via vortex, then 30 minutes of incubation at 37° C. The liposomes are then incubated with platelets at 37° C. for 30 minutes. Cells are washed once via centrifugation to remove incorporated liposome encapsulated doxorubicin or free doxorubicin. Drug loaded platelets can be lyophilized in appropriate buffer to create Thrombosomes. Flow cytometry and fluorescence microscopy may be performed to assess drug loading and intracellular localization. A fluorescence microplate reader can be used to obtain quantification of drug load. Light transmission aggregometry will be used to evaluate platelet function post drug load.

Endocytosis is a process through which a cell takes in material from its surroundings. The cell invaginates its plasma membrane to wrap around fluid or particles in its immediate environment. The internalized vesicle buds off from the plasma membrane and remains inside the cell.

Co-incubation of platelets with drug of interest occurs at 37° C. for 1-4 hours during which drug is loaded into platelets via endocytosis. Loaded platelets may then be lyophilized to make Thrombosomes. Loaded drug is detected via flow cytometry or fluorescence microscopy, provided drug is fluorescently tagged or is itself fluorescent. Loaded drug can be detected by HPLC or a microplate reader (e.g., a Tecan plate reader). Endocytic inhibitors such as amiloride (1 mM), phenylarsine oxide (10 μM), cytochalasin D (4 μM), or dynasore (25 μm) can be used to confirm that platelet loading is achieved by endocytosis.

Pep-1 is a 21 amino acid cell penetrating peptide with a C-terminal cysteamine group that shuttles cargo such as proteins or peptides into target cells. Pep-1 consists of a hydrophobic domain linked to a hydrophilic domain. The hydrophobic, tryptophan-rich domain can associate with a target cell membrane and the hydrophobic domains of the cargo protein (See e.g., Heitz, F., et. al., Twenty years of cell-penetrating peptides: from molecular mechanisms to therapeutics, British Journal of Pharmacology, 157, 195-206, (2009), which is incorporated herein by reference in its entirety).

The Pep-1 and the cargo protein are complexed by co-incubation at 37° C. for 30 minutes. The Pep-1: protein complex is incubated with platelets at 37° C. for at minimum 1 hour to allow Pep-1 mediated loading of protein cargo into the platelet. Platelets are washed by centrifugation to remove cell-free Pep-1: protein complex. Loaded platelets may then be lyophilized to make Thrombosomes. Platelets that have accumulated Pep-1 can be detected via flow cytometry or fluorescence microscopy if a fluorescent tag is attached to the C-terminus cysteamine of Pep-1. If the cargo protein is fluorescently labeled, then platelets containing this cargo may also be detected using flow cytometry or fluorescence microscopy.

The HIV Tat protein is another example of a cell penetrating peptide. The Tat protein includes between 86 and 101 amino acids depending on the subtype. Tat is a regulatory protein that enhances the viral transcription efficiency. Tat also contains a protein transduction domain which functions as a cell-penetrating domain allowing Tat to cross cellular membranes.

Lipofectamine is a cationic lipid; the Lipofectamine positively charged head group interacts with the negatively charged phosphate backbone of nucleic acids to facilitate transfection. Cellular internalization of the nucleic acid is achieved by incubating cells with the complexed Lipofectamine and nucleic acid.

Prepare the Lipofectamine and nucleic acid complex in aqueous buffer at room temperature. Incubate the complexed Lipofectamine and nucleic acid with platelets for 2-3 hours. Transfected platelets may be lyophilized to create Thrombosomes. Fluorescently labeled nucleic acid can be detected via flow cytometry and visualized using fluorescence microscopy. This method of loading is applicable to both RNA and DNA.

An electroporation machine generates electrical pulses which facilitate formation of transient openings in plasma membranes. The increased plasma membrane permeability allows entry of large and/or charged cargo that would otherwise not enter the cell due to membrane barrier.

Perform electroporation of platelets in the presence of desired cargo. Cargos of interest can be detected by flow cytometry and fluorescence microscopy if they are fluorescently tagged.

The influx cell loading strategy harnesses osmosis to load cells with water soluble, polar compounds. Cells are initially placed in a hypertonic solution containing drug of interest. In this hypertonic solution, water will move out of the cell into solution while drug will move into the cell via pinocytosis. Following that, cells are placed in a hypotonic solution in which water will enter the cell, lysing the pinocytic vesicles and thereby releasing drug into the cytosol.

Incubate platelets in hypertonic loading medium containing drug compound at 37° C. for at least 1 hour. Isolate loaded platelets from solution via centrifugation, resuspend platelets in hypotonic lysis medium, and incubate at 37° C. Pinocytic vesicles will burst and release drug into the cytosol. Fluorescently labeled drug can be visualized using fluorescence microscopy to confirm internalization. Flow cytometry may be performed to quantify drug load per cell for fluorescent drug.

Viral vectors are commonly used for transduction of cells. The host cell is driven by the viral vector to express the protein of interest at high load.

Use lentiviral vector to transfect 293T cells to generate pseudovirus, which is collected from the supernatant of this cell culture. The pseudovirus is then used to transduce megakaryocytes. Inside the transduced megakaryocyte, viral core plasmid containing cytomegalovirus promotor drives overexpression of the protein of interest, which gets packaged into platelets that bud off from transduced megakaryocytes.

Human platelets express FcγRIIA receptor which binds to the Fc region of IgG and facilitates internalization of IgG immune complexes. This method of loading platelets provides a route for delivery of therapeutic antibodies.

Incubate fluorescently labeled IgG at 62° C. for 20 minutes to prepare IgG immune complexes. Incubate IgG immune complexes with platelets for 1 hour at 4° C. to allow cells to bind immune complexes. Next, incubate immune complex-bound platelets at 37° C. to allow internalization of immune complexes. Flow cytometry can detect internalized fluorophore labeled IgG immune complexes. An anti-IgG-PE antibody specific for immune complexes can be used to identify surface bound, but not internalized, IgG-FITC immune complex.

Examples of drugs and of loading agents are as follows:

| | | Osmotic |
|---|---|---|
| | | hypertonic/ |
| | Cell penetrating | hypotonic |
| Endocytosis | peptide | loading |
| Dextran 10K | Dextran 10K | Dextran 10K |
| Dextran 500K | Dextran 3K | Dextran 3K |
| FITC-Albumin | Dextran 500K | Dextran 500K |
| FITC-Bovine IGG | FITC-albumin | FITC-albumin |
| FITC-F(ab)2 | FITC-Bovine IGG | FMLP |
| Histone H1 | FITC-F(ab)2 | Histone H1 |
| Lucifer yellow-slow uptake | FMLP | Lucifer Yellow |
| PE | Histone H1 | PE |
| | | (PHYCOERYTRIN) |
| Rabbit IGG | Lucifer yellow | Rabbit IGG |
| Soybean Trypsin Inhibitor | PE | Soybean Trypsin |
| | | Inhibitor |
| Doxorubicin | Rabbit IGG | Doxorubicin |
| Olaparib | Soybean Trypsin | |
| | Inhibitor | |
| Paclitaxel | | |

In some embodiments, the loading step comprises the use of dextran as a lyophilizing agent. In some embodiments the drug is an antibody. In some embodiments when the drug is an antibody, the drug is labeled with FITC (fluorescein isothiocyanate or 3′,6′-dihydroxy-6-isothiocyanatospiro[2-benzofuran-3,9′-xanthene]-1-one).

In some embodiments, drug-loaded platelets, drug-loaded platelet derivatives, or drug-loaded thrombosomes may shield the drug from exposure in circulation, thereby reducing or eliminating systemic toxicity (e.g. cardiotoxicity) associated with the drug. In some embodiments, drug-loaded platelets, drug-loaded platelet derivatives, or drug-loaded thrombosomes may also protect the drug from metabolic degradation or inactivation. In some embodiments, drug delivery with drug-loaded platelets, drug-loaded platelet derivatives, or drug-loaded thrombosomes may therefore be advantageous in treatment of diseases such as cancer, since drug-loaded platelets, drug-loaded platelet derivatives, or drug-loaded thrombosomes facilitate targeting of cancer cells while mitigating systemic side effects. In some embodiments, drug-loaded platelets, drug-loaded platelet derivatives, or drug-loaded thrombosomes may be used in any therapeutic setting in which expedited healing process is required or advantageous. In some embodiments, the therapeutic indications for cargo to be loaded into platelets include, for example, targeted depletion of cancer cells with chemotherapy drugs and therapeutic or prophylactic treatment of bacterial infection at site of injury with antibiotics.

In some embodiments, provided herein is a method of treating a disease as disclosed herein, comprising administering drug-loaded platelets, drug-loaded platelet derivatives, or drug-loaded thrombosomes as disclosed herein. In some embodiments, provided herein is a method of treating a disease as disclosed herein, comprising administering cold stored, room temperature stored, cryopreserved thawed, rehydrated, and/or lyophilized platelets, platelet derivatives, or thrombosomes as disclosed herein.

Examples of diseases (therapeutic indications) that may be treated with the drug-loaded platelets are as follows:

Therapeutic indications

Acute lymphoblastic leukemia (ALL)
Acute myeloid leukemia (AML)
Breast cancer (can also be used as an adjuvant
therapy for metastasized breast cancer post-surgery)
Gastric cancer
Hodgkin lymphoma
Neuroblastoma
Non - Hodgkin lymphoma
Ovarian cancer
Small cell lung cancer
Soft tissue and bone sarcomas
Thyroid cancer
Transitional cell bladder cancer
Wilms tumor
Cancer

Examples of cargo and therapeutic indications for cargo(s) to be loaded into platelets are as follows:

| Cargo | Therapeutic indications |
|---|---|
| Chemotherapy drug | Acute lymphoblastic leukemia (ALL) |
| (e.g., DOX, Olaparib, | Acute myeloid leukemia (AML) |
| Paclitaxel) | Breast cancer (can also be used as an adjuvant |
| | therapy for metastasized breast cancer post- |
| | surgery) |
| | Cancer |
| | Gastric cancer |
| | Hodgkin lymphoma |
| | Neuroblastoma |
| | Non - Hodgkin lymphoma |
| | Ovarian cancer |
| | Small cell lung cancer |
| | Soft tissue and bone sarcomas |
| | Thyroid cancer |
| | Transitional cell bladder cancer |
| | Wilms tumor |

In some embodiments, a drug may be fluorescent or labeled with a fluorescent moiety. For such a fluorescent or labeled drug, a correlation may be established between the fluorescence intensity and its concentration, and such a correlation may then be used to determine the concentration of the drug over a range of values. For example, FIG. 7 illustrates the correlation between fluorescence and concentration for doxorubicin. As FIG. 7 shows, the value of the concentration for doxorubicin in μM is determined according to the equation

X=(Y+344.92)/996.6

where Y is the fluorescence and X is the concentration.
An analogous correlation can be derived for the value of the amount of doxorubicin in mg:

mg of doxorubicin=concentration(μmol/L)×543.42 g/mol×50 μl/well/10⁹

or

mg of doxorubicin=(#μmol DOX/L)(mol/10⁶ μmol)(543.52 g/mol)(10³ mg/g)(L/10³ ml)(1 ml/10³ μl)(50 μl/well)

An analogous correlation can be derived for the value of the amount of doxorubicin in mg/cell:

mg/cell=mg(Intracellular+Membrane-bound doxorubicin)/total #of cells in a well.

Thus, the concentration of doxorubicin may be quantified from its excitation/emission spectra.

Examples of loading buffer that may be used are shown in Tables 1-4:

TABLE 1
Loading Buffer

| Component | Concentration (mM unless otherwise specified) |
|---|---|
| NaCl | 75.0 |
| KCl | 4.8 |
| HEPES | 9.5 |
| NaHCO₃ | 12.0 |
| Dextrose | 3 |
| Trehalose | 100 |
| Ethanol | 1% (v/v) |

Table 1. Loading Buffer is used to load platelets via endocytosis at 37° C. with gentle agitation as sample is placed on a rocker. Adjust pH to 6.6-6.8

TABLE 2
Buffer A

| Component | Concentration (mM unless specified otherwise) |
|---|---|
| CaCl₂ | 1.8 |
| MgCl₂ | 1.1 |
| KCl | 2.7 |
| NaCl | 137 |
| NaH₂PO₄ | 0.4 |
| HEPES | 10 |
| D-glucose | 5.6 |
| pH | 6.5 |

Table 2. Buffer A is used for loading platelets with liposome encapsulated drug. Incubation done at 37° C. with gentle agitation as sample is placed on a rocker.

TABLE 3
Buffer B

| Component | Concentration (mMunless otherwise specified) |
|---|---|
| Buffer and Salts | Table 4 (below) |
| BSA | 0.35% |
| Dextrose | 5 |
| pH | 7.4 |

Table 3. Buffer B is used when incubating platelets with fluorophore conjugated antibodies for flow cytometry.
This incubation is done at room temperature in the dark.
Albumin is an optional component of Buffer B

TABLE 4
Concentration of HEPES and of Salts in Buffer B

| Component | Concentration (mM unless otherwise specified) |
|---|---|
| HEPES | 25 |
| NaCl | 119 |
| KCl | 5 |
| CaCl₂ | 2 |
| MgCl₂ | 2 |
| Glucose | 6 g/l |

In Table 4 the pH adjusted to 7.4 with NaOH

Albumin is an optional component of Buffer B

In some embodiments, drug-loaded platelets are prepared by incubating the platelets with the drug in a loading buffer having the components shown in the table below.

In some embodiments, the loading buffer has the components as listed above in Table 1.

In some embodiments, incubation is performed at 37° C. using a platelet to drug volume ratio of 1:2, using different incubation periods.

### Example 1. Doxorubicin-Loaded Platelets

Doxorubicin-loaded platelets were prepared by incubating the platelets with doxorubicin in a loading buffer having the components shown in Table 5. Protocol 1 (described below) was used.

The platelet concentration in the loading buffer was 200,000 platelets/μl. The loading buffer had the following components:

TABLE 5

| Component | Concentration (mM unless specified otherwise) |
|---|---|
| NaCl | 75.0 |
| KCl | 4.8 |
| HEPES | 9.5 |
| NaHCO3 | 12.0 |
| Dextrose | 3 |
| Trehalose | 0.1M |
| Ethanol | 1% (v/v) |

Incubation was performed at 37° C. using a platelet to drug volume ratio of 1:2, using different incubation periods.

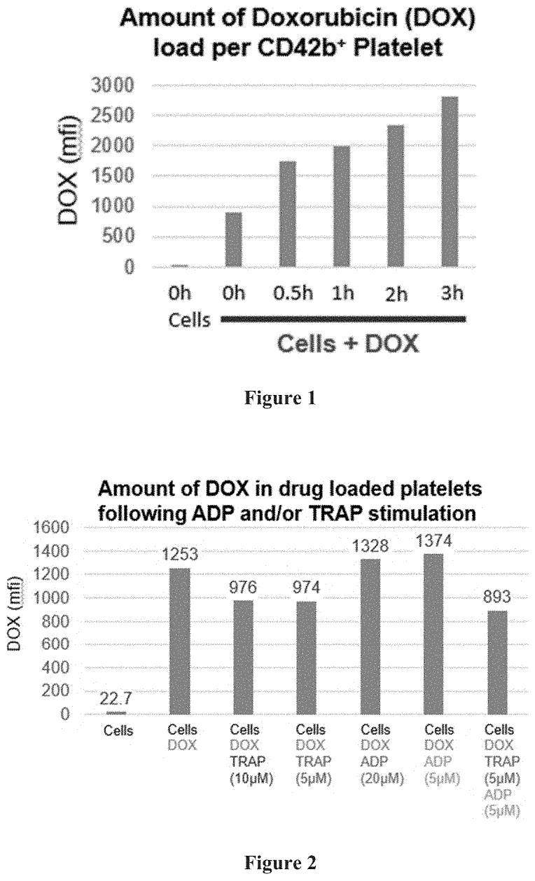

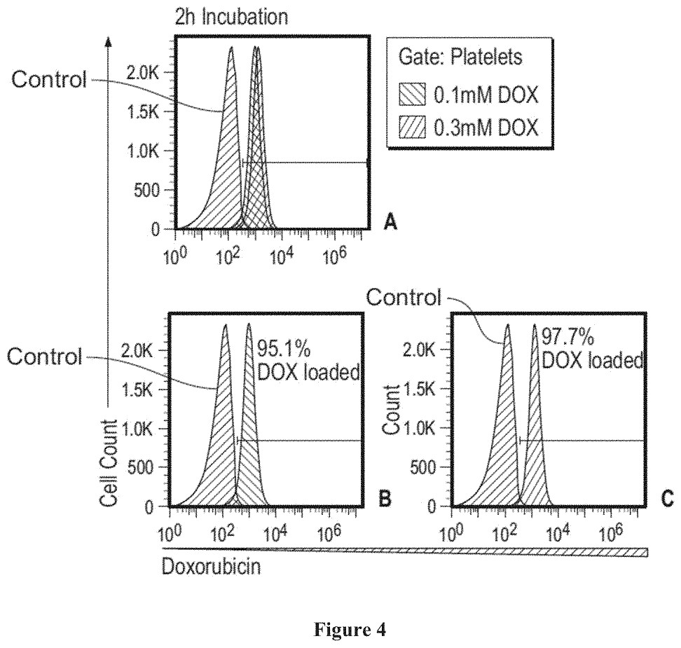

The drug-loading method was evaluated by flow cytometry to obtain a relative quantification of loading efficiency as mean fluorescence intensity of Doxorubicin in drug-loaded platelets. Platelets were evaluated for function by ADP and/or TRAP stimulation post-loading. The resulting amounts of doxorubicin load as a function of incubation time are provided in FIG. 1. Platelets were loaded with fluorescent DOX (excitation 470 nm, emission 560 nm) and evaluated by flow cytometry for fluorescence uptake. CD42b+ platelets load increasingly more DOX over time. % of platelets loaded with DOX is >90%, as shown in FIG. 4. DOX=doxorubicin. N=1.

In FIG. 2, DOX loaded platelets were incubated in loading buffer with ADP and/or TRAP for 10 minutes at room temperature to stimulate drug release. Following this incubation, flow cytometry was performed to assess decrease in drug load. DOX+ populations were gated on CD42b+ platelets. TRAP partially induced DOX release from drug-loaded platelets while ADP did not.

There is no synergistic effect of TRAP and ADP on DOX release from loaded platelets. ADP=adenosine diphosphate; TRAP=thrombin receptor activating peptide; DOX=doxorubicin. N=1.

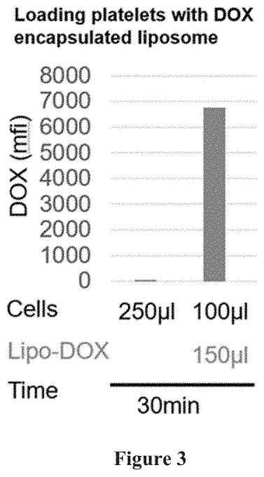

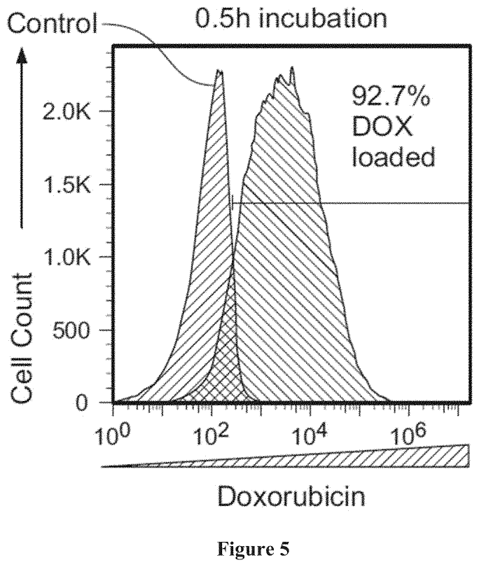

In FIG. 3, platelets (200,000 platelets/μl) were loaded with liposome encapsulated DOX (2 mg/ml). Liposome encapsulated DOX was prepared by rehydrating lyophilized lipid mixture (from Liposome Kit, Sigma-Aldrich, L4395) with 2 mg/ml DOX in PBS, followed by incubation at 37° C. for 30 minutes. Platelets and liposome encapsulated DOX were incubated at 37° C. for 30 minutes in Tyrode's HEPES buffer (Table 2). Flow cytometry was performed to obtain qualitative quantification of amount of DOX loaded per platelet. % of CD42b+ platelets loaded with liposome encapsulated DOX was >90% as shown in FIG. 5. N=1.

### Example 2. Time Course of Endocytic Loading Vs. Liposome-Mediated Loading of Doxorubicin into Platelets

The efficiency of Doxorubicin loading in to platelets via standard endocytosis or using liposome-encapsulated Doxorubicin (“Doxosomes”) was tested.

FIG. 4 provides data relating to endocytic loading of Doxorubicin into platelets. Platelets were incubated with 0.1 mM Doxorubicin or 0.3 mM Doxorubicin for two hours. Doxorubicin was allowed to enter the platelets via endocytosis. The top graph shows amalgamated data that includes the bottom left graph (Doxorubicin at 0.1 mM) and the bottom right graph (Doxorubicin at 0.3 mM). Doxorubicin was efficiently loaded after two hours of incubation at concentrations of both 0.1 mM and 0.3 mM. For each graph, the left-most curve represents cells only.

FIG. 5 provides data relating to liposome-mediated loading of Doxorubicin into platelets. Platelets were incubated with 13.5 μM of Doxorubicin-containing doxosomes for 0.5 hours. Briefly, a total of 90 μmol of lipids (63 μmol L-A-Phosphatidylcholine, 18 μmol Stearylamine, and 9 μmol Cholesterol) were resuspended in 1 mL of Doxorubicin in PBS to generate the doxosomes. 150 μL of doxosome containing 12 mg/mL of liposome-encapsulated Doxorubicin was incubated with platelets to a final doxosome concentration of 13.5 μM. Doxorubicin was efficiently loaded after 0.5 hours of incubation with the doxosomes.

As can be seen from the Figures, loading of doxorubicin occurred efficiently both via doxosomes (FIG. 5) and using endocytotic loading (FIG. 4).

Exemplary protocols for the loading of platelets with doxorubicin are shown below:

Protocol 1. Loading Platelets with DOX Via Endocytosis

The starting apheresis platelet material was pooled and characterized. The platelet pool was acidified to pH 6.6-6.8 using Acid Citrate Dextrose solution. Platelets were isolated by centrifugation at ˜1500 g for 20 minutes, with slow acceleration and braking. The supernatant plasma was aspirated and disposed of.

The platelets were suspended in the loading buffer of Table 1 at a concentration of 200,000 platelets/μl. While the platelets were being centrifuged, a doxorubicin (“DOX”) solution having a concentration of 0.3 mM in the loading buffer was prepared as follows: a solution of DOX in water (50 mg/ml) was pre-warmed at 37° C. for 20 minutes; DOX was then incubated in the loading buffer at a concentration of 0.3 mM at 37° C. for 20 minutes, periodically subjecting it to a vortex.

The platelets and DOX (1 ml platelets at 200,000 platelets/μl+2 ml DOX at 0.3 mM) were then mixed and the mixture was incubated at 37° C. for 2 hours. The resulting DOX-loaded platelets were lyophilized.

Optionally, the lyophilized DOX-loaded platelets could be suspended in water at a concentration suitable for the uses disclosed herein.

Protocol 2. Loading Platelets with Liposome Encapsulated DOX

The starting apheresis platelet material was pooled and characterized. The platelet pool was acidified to pH 6.6-6.8 using Acid Citrate Dextrose solution. Platelets were isolated by centrifugation at ˜1500 g for 20 minutes, with slow acceleration and braking. The supernatant plasma was aspirated and disposed of.

The platelets were suspended in Buffer A at a concentration of 200,000 platelets/μl. The components of Buffer A are shown above Table 2.

TABLE 6
Tyrode's HEPES Buffer

| Component | Concentration (mM) |
|---|---|
| CaCl₂ | 1.8 |
| MgCl₂ | 1.1 |
| KCl | 2.7 |
| NaCl | 137 |
| NaH₂PO₄ | 0.4 |
| HEPES | 10 |
| D-glucose | 5.6 |
| pH | 6.5 |

While the platelets were being centrifuged, liposome-encapsulated doxorubicin (“DOX”) was prepared as follows: lyophilized phospholipids (Sigma-Aldrich, SKU #DOX-1000) were rehydrated with a 2 mg/ml solution of DOX in PBS; the rehydrated mixture was then subjected it to a vortex for 30 seconds and incubated at 37° C. for 30 minutes.

The platelets in Tyrode's HEPES buffer and the liposomal DOX were then mixed and the mixture was incubated at 37° C. for 30 minutes.

The resulting DOX-loaded platelets were washed in 1 mL Tyrode's HEPES buffer to remove unincorporated liposome by centrifugation at 1500 g for 20 minutes.

Optionally, the lyophilized DOX-loaded platelets could be suspended in water at a concentration suitable for the uses disclosed herein.

Protocol 3. Loading Platelets with Liposome Encapsulated DOX

This protocol was similar to Protocol 2 herein, except that the buffer was as follows instead of the buffer of Table 6 above:

TABLE 7
Tyrode's HEPES Buffer (plus PGE1)

| Component | Concentration (mM) |
|---|---|
| CaCl₂ | 1.8 |
| MgCl₂ | 1.1 |
| KCl | 2.7 |
| NaCl | 137 |
| NaH₂PO₄ | 0.4 |
| HEPES | 10 |
| D-glucose | 5.6 |
| pH | 6.5 |
| Prostaglandin E1 | 1 μg/ml |
| (PGE1) | |

Protocol 4. Loading Platelets with Liposome Encapsulated DOX

This protocol was similar to protocol 2 herein, except that the buffer of Table 1 was used (shown again below) instead of the buffer of Table 6 above:

### Experimental Results. Effect of Buffer on Liposome-Mediated Loading of Doxorubicin into Platelets

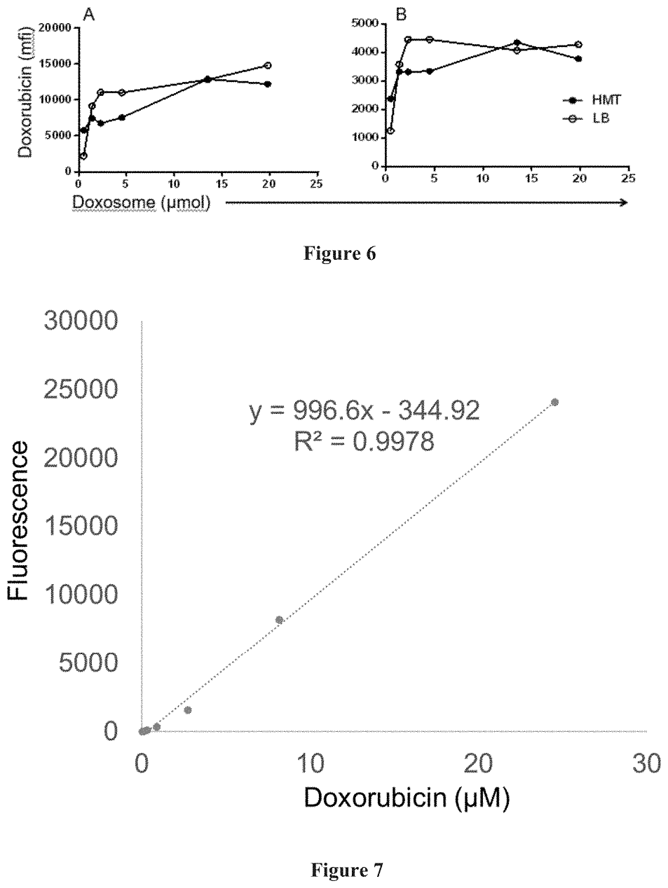

Platelets were incubated with doxosomes containing fluorescent doxorubicin as described above in Example 2 in the presence of trehalose-containing loading buffer (as shown in Table 1) or HMT (as shown in Table 7) As can be seen in FIG. 6, trehalose-containing loading buffer increased the total amount of Doxorubicin that was loaded as compared to HMT.

FIG. 6 provides a comparison of doxosome loading efficiency between conventional HMT buffer (Protocol 3, shown in continuous line) and trehalose-containing loading buffer (Protocol 4, shown in individual points) described herein. The x axis represents the amount of doxosome added to the platelets. The y axis represents mean-fluorescence intensity. The graph on the left (labeled as (A) in FIG. 6) provides the doxosome loading efficiency of loading platelets with CD42b antibodies to define the platelets while the graph on the right (labeled as (B) in FIG. 6) provides the doxosome loading efficiency of platelets without CD42b. A higher drug load is obtained with the trehalose-containing loading buffer of Protocol 4 as compared to the conventional HMT loading buffer at all points after about 20 minutes.

### Example 3—Determination of Amount of Doxorubicin (“DOX”) in a Platelet

### Protocol A:

Fresh donor platelets were provided. Incubation with the drug took place by endocytosis at 37 C for 3 hours in a rocker at low frequency, in the presence of a buffer containing HMT and 1 μM PGE1. The correlation between fluorescence and concentration is shown in FIG. 7.

### Calculations of Amounts—Example 3A-1

1) Concentration:
In a well containing 50 μL and having a concentration of 250K cells per μL, the intracellular doxorubicin fluorescence was 10811 and the membrane-bound doxorubicin fluorescence was 1263. Calculating the concentration from the formula

X=(Y+344.92)/996.6,
where Y is the fluorescence and X is the concentration, provides an intracellular concentration 11 μM and a membrane-bound concentration of 2 μM.

2) Quantity in Mg:
The amount in mg is calculated from the formula:

mg of doxorubicin=concentration(μmol/L)×543.42×50/10⁹

3) Quantity in Mg/Platelet:
The amount in mg/platelet is calculated from the formula:

(Intracellular DOX amount(per well)+membrane-bound DOX amount(per well))/total #of platelets in a well
For example, for an intracellular amount of 3.04×10⁻⁴ mg and a membrane-bound amount of 4.38×10⁻¹ mg, if there are 250,000 cells/μl and 50 μl/well, then the total amount will be 2.78×10⁻¹¹ mg/platelet.
The calculations have a percentage error of 7.16%, calculated as follows:
Intracellular concentration of doxorubicin: 11 μM
membrane-bound concentration of doxorubicin: 2 μM.
Soluble concentration of doxorubicin: 298 μM.
Total concentration of doxorubicin: 290 μM.

% error=[(298+11+2)−290)/290]×100%=7.16%

### Calculations of Amounts—Example 3A-2

1) Concentration:
In a well containing 50 μL and having a concentration of 125K cells per μL, the intracellular doxorubicin fluorescence was 7496 and the membrane-bound doxorubicin fluorescence was 438. Calculating the concentration from the formula

X=(Y+344.92)/996.6,
where Y is the fluorescence and X is the concentration, provides an intracellular concentration 8 μM and a membrane-bound concentration of 1 μM.

2) Quantity in Mg:
The amount in mg in a 50 μL sample is calculated from the formula:

mg of doxorubicin=concentration(μmol/L)×543.42×50/10⁹

3) Quantity in Mg/Platelet:
The amount in mg/platelet is calculated from the formula:

(Intracellular DOX amount+membrane-bound DOX amount)/total #of platelets in a well
For example, for an intracellular amount of 2.14×10⁻⁴ mg and a membrane-bound amount of 2.13×10⁻⁵, if there are 125,000 cells/μl and 50 μl/well, then the total amount will be 1.88×10⁻¹¹ mg/platelet.
The calculations have a percentage error of 1.94%, calculated as follows:
Intracellular concentration of doxorubicin: 8 μM
membrane-bound concentration of doxorubicin: 1 μM.
Soluble concentration of doxorubicin: 295 μM.
Total concentration of doxorubicin: 310 μM.

% error=[(295+8+1)−310)/310]×100%=1.94%

### Protocol B:

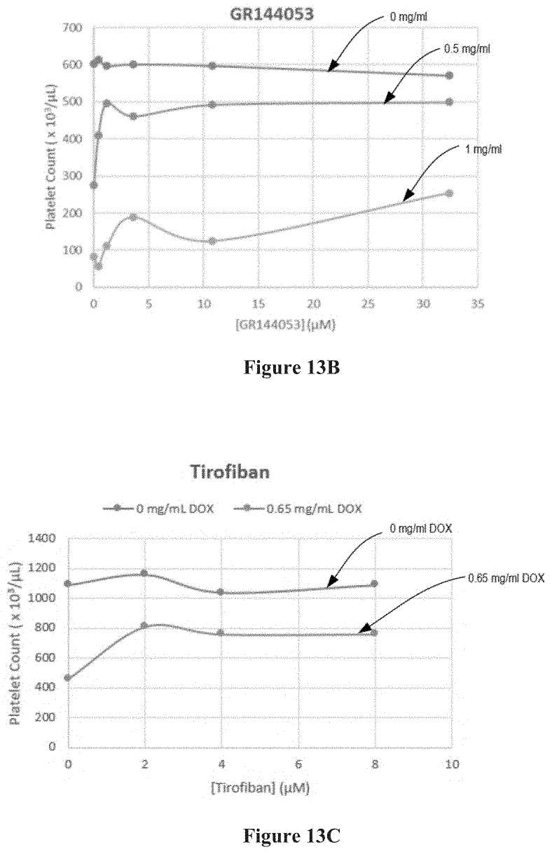

Four apheresis units were pooled. Incubation with the drug took place at 37 C for 4 hours in a rocker at low frequency, in the presence of a buffer containing HMT and 1 μM PGE1. The correlation between fluorescence and concentration is shown in FIG. 13.

### Calculations of Amounts—Example 3B-1

1) Concentration:
Calculations were performed in a manner analogous to Example 3A-1 and 3A-2 above, the correlation between fluorescence and concentration was based on a standard curve.
The resulting quantity of DOX in mg/platelet for various values of cells/μl is as follows:

| Cells/μl | DOX (mg/cell) |
|---|---|
| 108K | 4.4 × 10⁻⁶ |
| 235K | 3.2 × 10⁻⁶ |
| 454K | 2.2 × 10⁻⁶ |
| 915K | 1.5 × 10⁻⁶ |
| 1448K | 1.0 × 10⁻⁷ |
| 1745K | 9.3 × 10⁻⁷ |

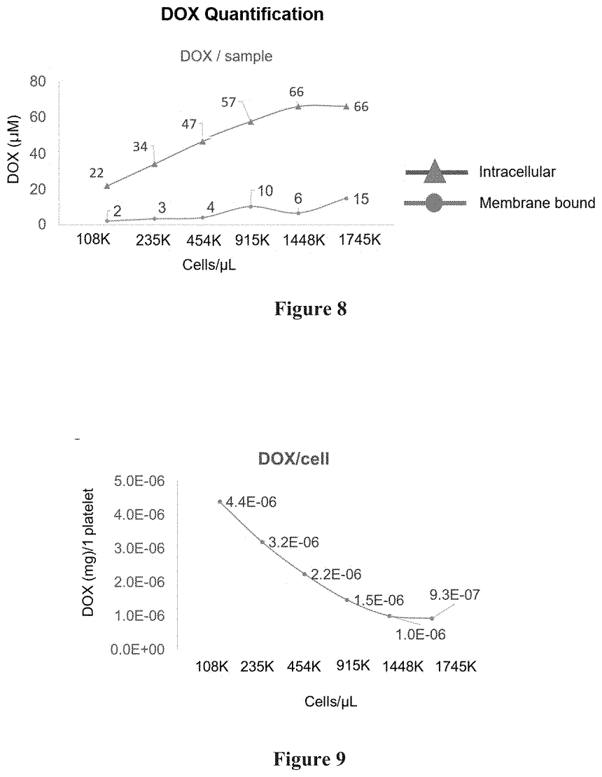

FIGS. 8 and 9 are based on the same data showing that the total amount of DOX loaded into platelets increases as a function of increasing platelet count, but the amount of DOX loaded into an individual platelet decreases.

FIG. 8 shows the total concentration of (a) intracellular doxorubicin, and (b) membrane-bound doxorubicin, with increasing concentration of platelets/μL.

FIG. 9 shows how for a given concentration of doxorubicin (DOX) (0.2 mM), the amount of DOX/platelet decreases as the number of platelets increases.

### Example 4. Comparison of Amount of Drug Loaded with Loading Buffer and with HMT Buffer

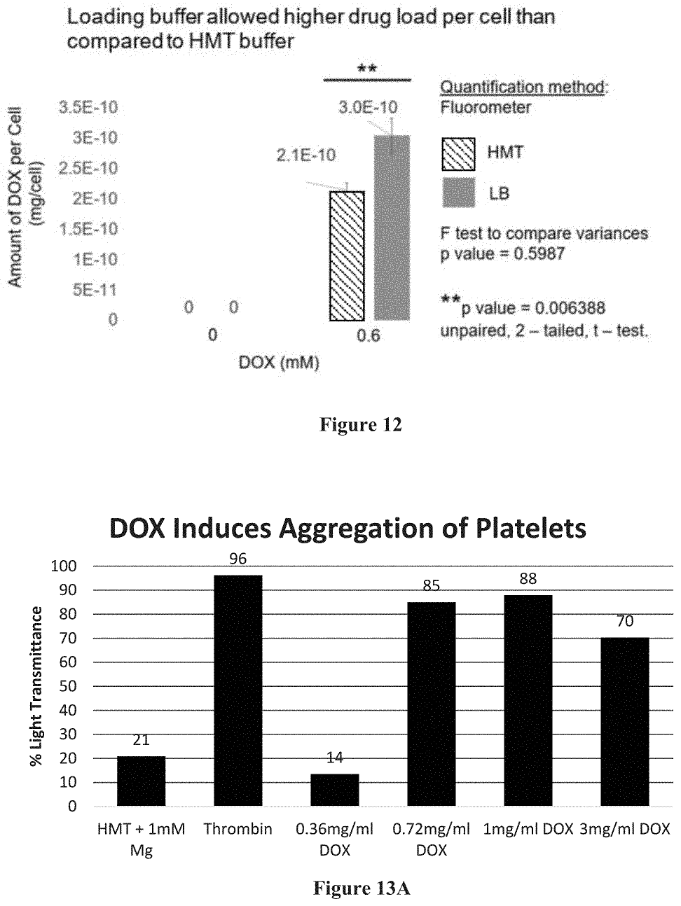

Platelets pooled from eight apheresis units, at a concentration of (250,000 cells/μL, were incubated in either the Loading Buffer of Table 1 (LB in FIG. 12) or HMT containing PGE₁ (1 μM) (Table 7) in the presence of DOX (0.6 mM or 0.36 mg/ml) compared to a control with no DOX for 3 hours at 37° C. Following incubation, platelets were isolated from buffer via centrifugation (Beckman Coulter Microfuge 18 Centrifuge, 845×g, 10 minutes, room temperature), then washed twice with LB or HMT using the same centrifugation setting as stated. After that, the platelets were sonicated 3 times to release intracellular DOX at 26 kHz for 30 seconds with 2-5 minutes interval of rest at room temperature. These samples are then centrifuged (Eppendorf Centrifuge 5424, 18,000 G, 20 minutes, room temperature) to separate intracellular DOX (supernatant) from membrane bound DOX (pellet). The pellet was resuspended in 1 ml of buffer (HMT or LB) then sonicated 3 times at previously stated setting to generate homogenized sample of membrane bound DOX in buffer. Quantification of DOX is achieved with 500 nm excitation and 600 nm emission using the TECAN Infinite M200 PRO. A 96-welled polystyrene, half area, non-treated, black with clear flat bottom plate (Corning) is used in which 50 μL of sample is plated per well in triplicates. DOX load per platelet (mg/cell) is calculated based on standard curve (linear regression, R²=0.9873 for cell lysate in HMT, R²=0.9902) generated from serial dilution of cell lysate. As shown in FIG. 12, the loading buffer allowed higher drug load per cell than compared to HMT buffer.

### Example 5—Induced Drug Release from Doxorubicin-Loaded Platelets

Pooled apheresis platelets from between 8 and 11 apheresis units (defined as 200-400 mL of plasma from a single donor) were diluted to 250,000 cells/μL. A test group included 0.2 mM doxorubicin (DOX) and a control group contained no agonist. Both the test and control groups were incubated in a HMT buffer of pH 6.8 containing 1 μM Prostaglandin E1 (PGE1) for 3 hours at 37° C.

Any excess drug not loaded into the platelet was removed by subjecting the test group to centrifugation at 800×g for 10 minutes at room temperature and by discarding the supernatant. The drug-loaded cells of the test group were resuspended to the same concentration in HMT buffer at pH 7.4 then stimulated with agonists including collagen, ADP, thrombin, arachidonic acid (AA) and thrombin receptor activating peptide (TRAP-6) for 15 minutes at room temperature. The supernatant was harvested by centrifugation as described above.

The control group for intracellular drug was prepared by three rounds of vibration sonication for 30 seconds at 20 kHz prior to centrifugation.

Fifty μl of supernatant of the test and control groups was applied to fluorescent compatible clear flat bottom 1%2 area of 96 well micro-titer plates and analyzed on a Tecan Sapphire plate reader with excitation at 500 nm and emission at 600 nm and the released Doxorubicin quantified relative to a standard curve obtained under the same conditions.

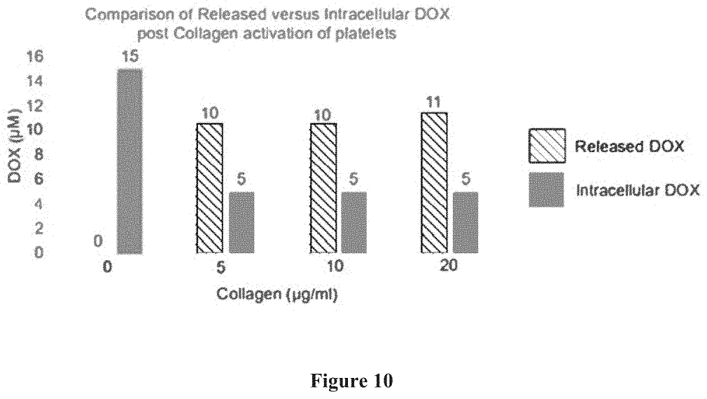

The amount of released and intracellular DOX in DOX-loaded platelets when stimulated with varying amounts of collagen are shown in FIG. 10. The effect of varying amounts of collagen on inducing drug release on DOX-loaded platelet, as measured by light excitation and emission, is shown in FIG. 10 in comparison with a control group containing no collagen.

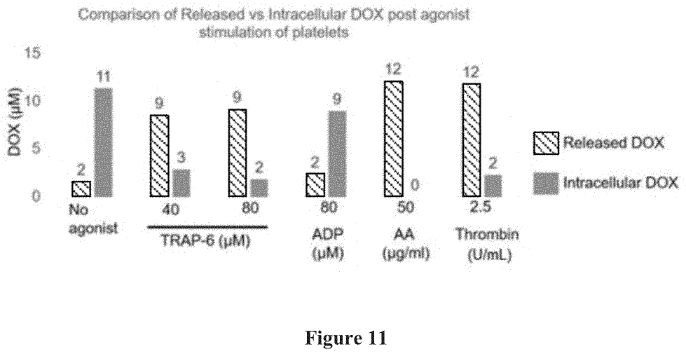

The amount of released and intracellular DOX in DOX-loaded platelets when stimulated with TRAP-6, ADP, AA, and thrombin are shown in FIG. 11. The effect of different agonists on inducing drug release on DOX-loaded platelet, as measured by light excitation and emission, is shown in FIG. 11 in comparison with a control group containing no agonist.

### Example 6—Loading Buffer Allows Higher Drug Load Per Cell than HMT Buffer

FIG. 12 shows a higher drug load per cell when using loading buffer as compared to HMT buffer. The amount of DOX is measured in mg/cell by a fluorometer.

### Example 7—DOX Induced Aggregation of Platelets

Loading platelets with DOX causes the platelets to aggregate resulting in a decrease in platelet count. The extent of platelet aggregation is measured as a function of the transmittance of light through a stirred suspension of platelets on an AggRAM. Platelets as single cells in a suspension are too turbid for light to effectively pass through. However, when platelets aggregate they fall out of suspension and allow more light to pass through, thus increasing the transmittance. FIG. 13A shows the DOX induced platelet aggregation measured by percent light transmittance.

Platelets were treated with 0.36 mg/ml, 0.72 mg/mL, 1 mg/mL, and 3 mg/ml of DOX. Thrombin was used as a positive control. HMT and 1 mM magnesium was used as a negative control (FIG. 13A).

The concentration of platelets (count) is determined on an AcT-Diff hematology analyzer. As shown in FIG. 13B-C, the platelet count decreases with increasing DOX concentration after incubation for 3 hours. The only anti-platelet compounds that shows effective inhibition of DOX-induced aggregation in this assay were the GPIIb/IIIa inhibitors GR144053 and Tirofiban (See Table 1)

FIG. 13B shows that a GPIIb/IIIa inhibitor GR 144053 (>1.2 μM) limited reduction of platelet count following coincubation with 0.5 mg/ml DOX for 1 hour at 37° C. The data were generated from platelets pooled from seven apheresis units. Platelets (600,000 cells/μL), DOX (0 mg/ml top line, 0.5 mg/ml middle line, 1 mg/ml bottom line), and GR 144053 (0, 0.4, 1.2, 3.6, 10.8, and 32.4 μM. Tocris Bioscience Cat. #1263.) were incubated in loading buffer for 1 hour at 37° C. Following incubation, platelets were quantified via Coulter AcoT diff2 Hematology Analyzer (×103/μL).

### Example 8—Platelets Retain DOX after Cryopreservation or Lyophilization

The concentration of Dox loaded into platelets remains the same after cryopreservation. Platelets were incubated with 600 μM Dox in the presence of PGE1 and GR144053 at 37° C. for 3 hours, then washed, and subjected to cryopreservation in loading buffer containing 6% polysucrose.

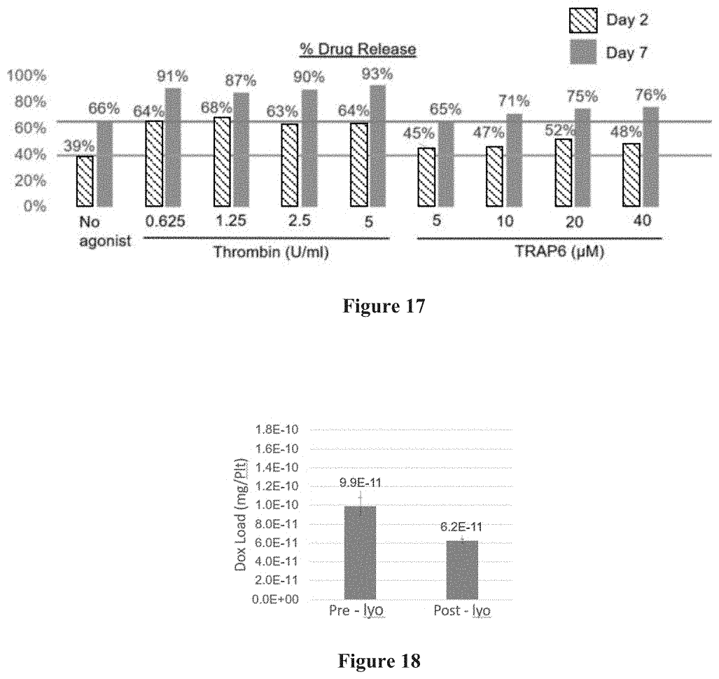

After cryopreservation, DOX can be released from within platelets in response to strong platelet activating agents described herein. The released DOX concentration was measured by fluorescence on a plate reader after 2 (left hand columns) and 7 (right hand columns) days after incubation and centrifugation (FIG. 17). The released DOX is shown here as a percentage of the total DOX measured before cryopreservation. The bottom line marks the DOX release without agonist at 2 days and the top line marks the DOX release without agonist after 7 days.

A series of quality control experiments were performed with DOX loaded thrombosomes. The batch is a large batch measured in apheresis units. The batch contains Sublot A with 98 vials of unloaded thrombosomes and Sublot B with 98 vials of DOX-loaded thrombosomes.

DOX-loaded thrombosomes were prepared by incubation with 600 μM DOX then following thrombosomes manufacturing specifications. The concentration of DOX loaded into platelets was measured by fluorescence after sonication as described herein. FIG. 18 shows the concentration of DOX loaded into platelets before and after lyophilization to a dry powder and rehydration was found to be >43% of the concentration measured immediately prior to lyophilization.

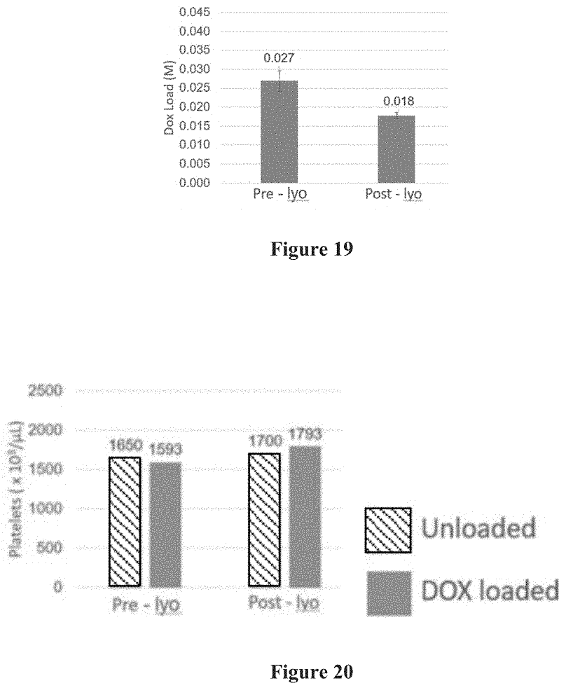

FIG. 19 represents the same data from FIG. 18, but the data were converted to molar concentration by using the mean platelet volume (MPV) after DOX loading. FIG. 19 shows that the intracellular DOX concentration is increased 50-fold prior to lyophilization and maintained at 30-fold after lyophilization relative to the external DOX concentration during incubation.

FIG. 20 shows that platelet counts, as measured by the AcT-Diff hematology analyzer, remain mostly unchanged after lyophilization. The target platelet count when preparing the platelet suspensions for lyophilization was 2000×10³/μL, and should remain similar after lyophilization and rehydration in the same volume used to aliquot the platelet suspensions prior to lyophilization.

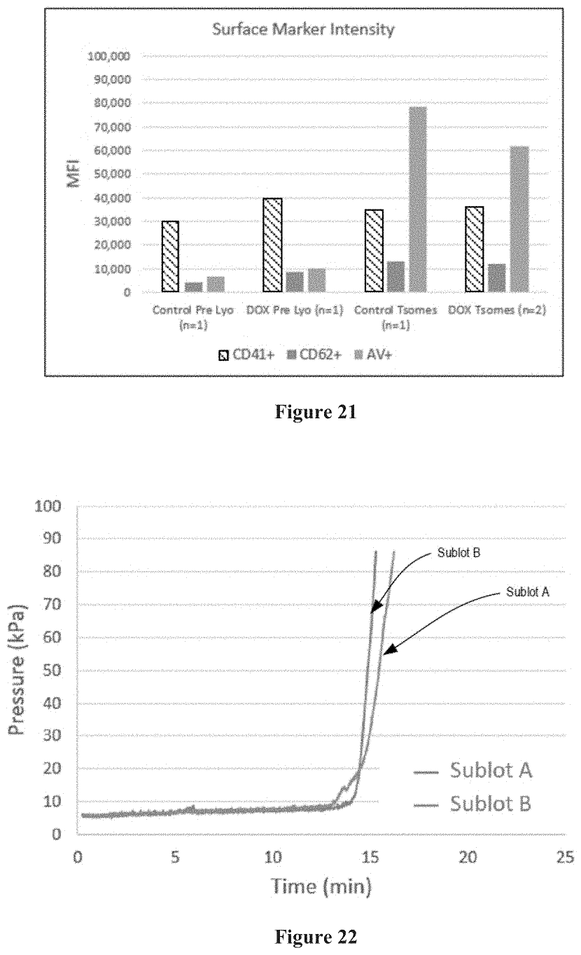

FIG. 21 shows that DOX-loaded thrombosomes have similar platelet receptor biomarker expression relative to unlabeled thrombosomes immediately prior to and after lyophilization. CD41 (left hand columns), CD62 (middle columns), and Annexin V (right hand columns) expression levels were measured using fluorescently-labeled antibodies for each receptor. The fluorescent labels were chosen such that DOX fluorescence would not interfere with the antibody fluorescence signal.

FIG. 22 shows the batch containing Sublot A with 98 vials of unloaded thrombosomes and Sublot B with 98 vials of DOX-loaded thrombosomes adhere to collagen and tissue factor coated microchips. Specifically, DOX-loaded thrombosomes adhere to collagen and tissue factor coated microchips similar to unloaded thrombosomes. The occlusion times were determined on a T-TAS by measurement of the pressure while suspensions of thrombosomes were flowed over coated surfaces.

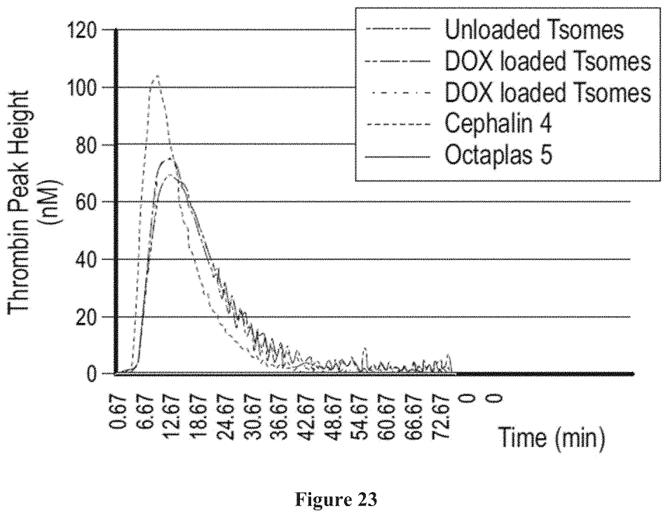

FIG. 23 shows that DOX-loaded thrombosomes generate thrombin similar to unloaded thrombosomes. Thrombin generation was determined by the measurement of fluorescent signal from a substrate that does not fluoresce until cleavage by thrombin, relative to a control. Cephalin is a positive control to show the maximum possible thrombin generation in the assay, and octaplas is negative control unable to generate thrombin.

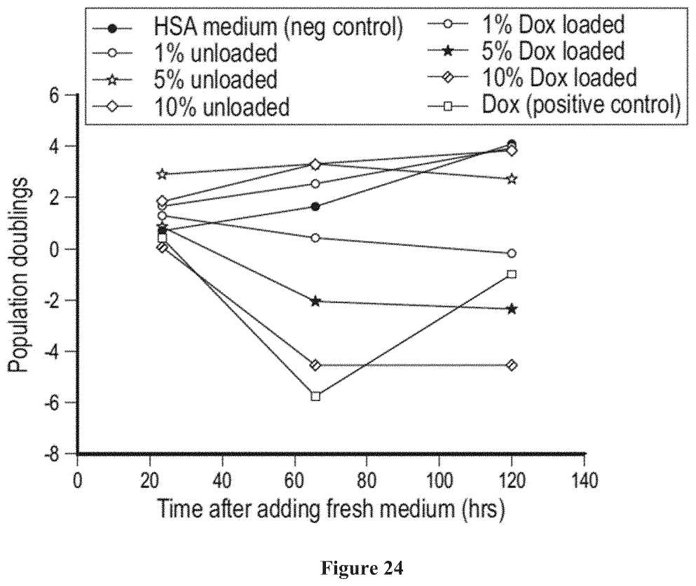

FIG. 24 shows that DOX loaded thrombosomes can inhibit cancer cell growth more effectively than DOX alone. Dog hemangiosarcoma cells (DHSA) (Kerafast, DHSA-1426, Cat. #EMN017) were plated at a fixed density in triplicate in 24-well plates and grown overnight. The following day, the loaded and unloaded thrombosomes were rehydrated as described herein and assayed within one hour. The DHSA cells were exposed to medium without thrombosomes (HAS) as a negative control and 1%, 5%, or 10% unloaded thrombosomes and 1%, 5%, or 10% DOX loaded thrombosomes, or free DOX (in medium, 5 uM). The next day after about 24 hours of exposure, the test medium was removed by gentle aspiration, the plates were washed once with fresh medium, and fresh medium was added. The point at which fresh medium was added is considered time zero (FIG. 24). At day 1 (24 hours), 3 (72 hours) and 5 (120 hours), a 24 well plate was harvested. The triplicate wells were pooled and counted using the Nexcelom cell counter Auto 2000 with AO/PI live/dead cell count.

### Example 9—Olaparib-Loaded Platelets

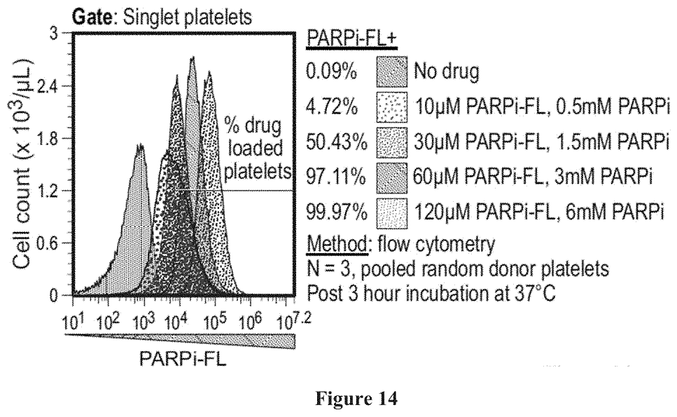

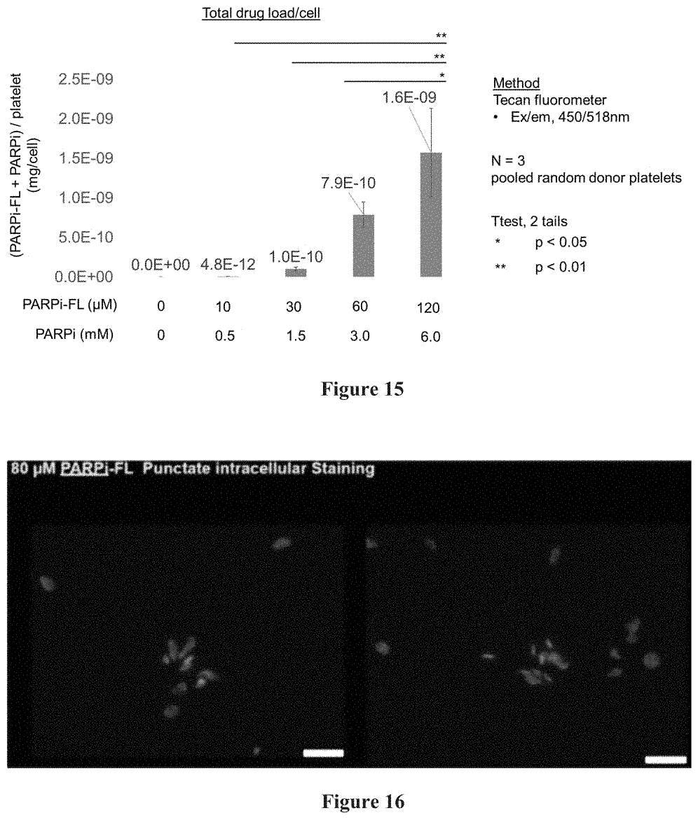

Poly ADP ribosome polymerase (PARP) inhibitors are drugs used in the treatment of cancer. PARP inhibitors interfere with a cell's ability to repair DNA breaks. Olaparib (also known as AZD-2281, MK-7339, or Lynparza®) is a PARP inhibitor. FIG. 14 shows that Olaparib loads into platelets in a dose-dependent manner. After incubation with increasing total concentrations of PARPi (Olaparib) and a fluorescently labeled PARPi-FL, keeping the ratio at 20:1, platelets were analyzed by flow cytometry. PARPi and PARP-FL were tested at the following concentrations: 10 μM PARPi-FL and 0.5 mM PARPi, 30 μM PARPi-FL and 1.5 mM PARPi, 60 μM PARPi-FL and 3 mM PARPi, and 120 μM PARPi-FL and 6 mM PARPi. The percentage of loaded PARPi-loaded platelets increases with increasing concentrations as measured by the mean fluorescence intensity. FIG. 15 shows the total drug load increase following incubation of cells with increasing concentrations of olparib. The platelets were sonicated and the intracellular fluorescence signal was measured on a plate reader.

Olaparib loaded platelets were verified by loading microscopy. FIG. 16 shows a fluorescence image. Olaparib is localized within platelets. After incubation with 80 μM PARPi-FL at 37° C. for 3 hours, platelets were washed to remove excess fluorophore and fixed on microscope slides at 4° C. overnight. Images were collected on a fluorescence microscope at 100× magnification. These images show the internalization of PARPi into platelets.

### Example 10—Paclitaxel-Loaded Platelets

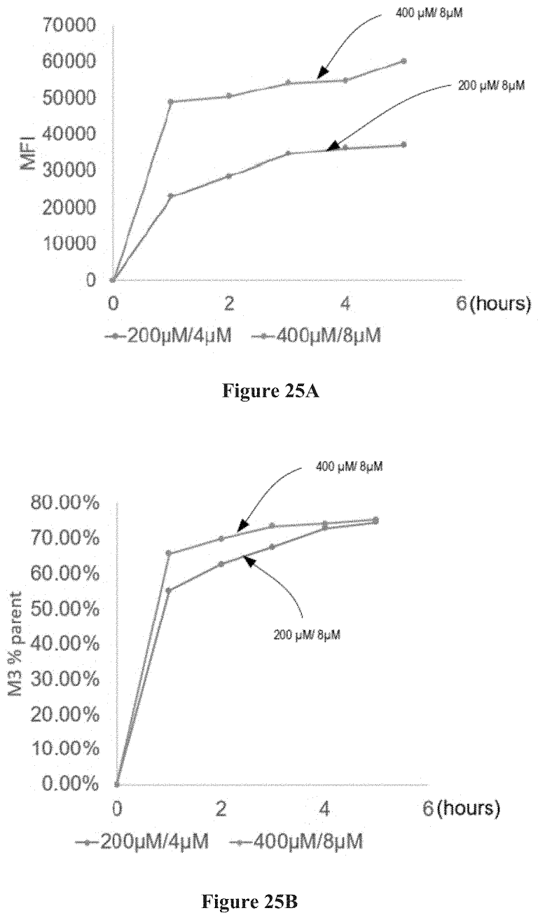

Paclitaxel is a chemotherapy agent that interferes with the normal function of microtubules during cell division. Paclitaxel is loaded into platelets in a dose-dependent manner. FIG. 25A and FIG. 25B show incubation of platelets with increasing total concentrations of Paclitaxel and fluorescently labeled Paclitaxel-Oregon Green. The platelets were kept at a ratio of 50:1, unlabeled drug to labeled drug. Uptake of paclitaxel was measured by flow cytometry. Measurements were taken at 2, 4, and 6 hours. As time increased from 0 to 6 hours the concentration increased as measured by MFI and percent loaded increases.

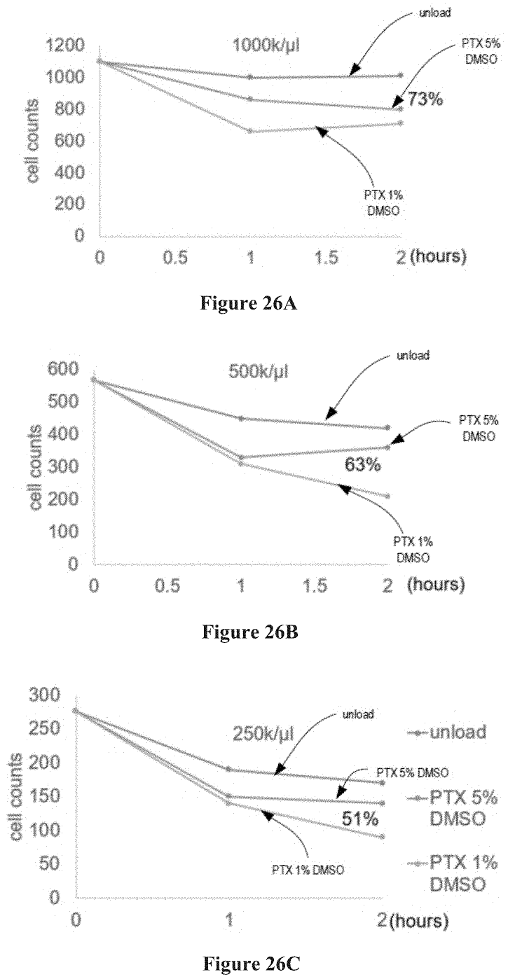

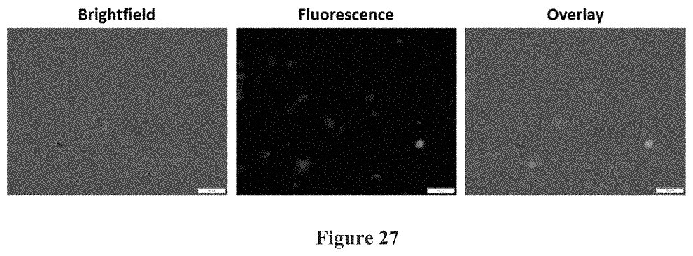

FIGS. 26A-FIG. 26C show the concentration of platelets (measured by count) is determined on an AcT-Diff hematology analyzer during incubation with Paclitaxel. The platelet count is maintained as a function of increasing initial platelet concentration in the presence of paclitaxel, irrespective of the DMSO concentration. Minimally 50% of platelets (platelet count) are retained after loading.

Paclitaxel loaded platelets were verified by loading microscopy. FIG. 27 shows brightfield and fluorescence images, followed by an overlay image. Paclitaxel is localized within platelets. After incubation with 100 μM Paclitaxel-Oregon Green at 37° C. for 2 hours, platelets were washed to remove excess fluorophore and fixed on microscope slides at 4° C. overnight. Images were collected on a fluorescence microscope at 100× magnification. FIG. 27 shows the internalization of Paclitaxel into platelets.

### Example 11. siRNA-Loaded Platelets

Stored and fresh platelets were used to test siRNA loading. Stored platelets were collected via the apheresis method in an acid-citrate-dextrose (ACD) solution and stored in blood bags for 24 hours before use. Fresh platelets were collected on the day-of-use in an ACD solution in BD Vacutainer® tubes and isolated from whole blood by centrifugal blood fractionation.

A FITC-conjugated siRNA control (BLOCK-iT™ Fluorescent Oligo, ThermoSci Cat. #2013) was used to assay siRNA-loading in both stored and fresh platelets. The siRNA was loaded into the platelets using a cationic lipid transfection agent (Lipofectamine™ RNAiMAX, ThermoSci) in HBS buffer (Table 8).

Liquid transfection media conditions were tested to assay siRNA-loading. Four different liquid transfection media conditions were tested on fresh platelets: Loading Buffer (Table 9), Loading Buffer without ethanol (Table 10), HEPES-buffered Tyrode's solution (HMTA, Table 11), and Opti-MEM™ (ThermoSci, commonly used cell growth and transfection medium). Stored platelets were tested with Loading Buffer supplemented with or without Opti-MEM™ (Table 12). The components of each buffer are shown in their respective tables. The siRNA-loaded platelets were prepared by incubating either the stored or fresh platelets with the BLOCK-iT™ Fluorescent Oligo (0.377 μM) and cationic transfection reagent (0.24% v/v) in one of the indicated buffers. Protocol 5 (described below) was used, except where otherwise indicated.

The platelet concentration in the various liquid transfection media conditions was 6.35×10⁷ platelets/mL.

TABLE 8
HBS:

| | Concentration |
|---|---|
| Component | (mg/mL) |
| NaCl | 8.77 |
| HEPES | 2.38 |
| pH | 6.6-6.8 |

TABLE 9
Loading Buffer:

| | Concentration |
|---|---|
| | (mg/mL, except where |
| Component | otherwise indicated) |
| NaCl | 4.38 |
| KCl | 0.36 |
| HEPES | 2.26 |
| NaHCO₃ | 1.01 |
| Dextrose | 0.54 |
| Trehalose | 34.23 |
| Ethanol | 1.00% (v/v) |
| pH | 6.6-6.8 |

TABLE 10
Loading Buffer without ethanol:

| | Concentration |
|---|---|
| | (mg/mL, except where |
| Component | otherwise indicated) |
| NaCl | 4.38 |
| KCl | 0.36 |
| HEPES | 2.26 |
| NaHCO₃ | 1.01 |
| Dextrose | 0.54 |
| Trehalose | 34.23 |
| pH | 6.6-6.8 |

TABLE 11
HTMA:

| | Concentration |
|---|---|
| | (mM, except where |
| Component | otherwise indicated) |
| CaCl₂ | 1.8 |
| MgCl₂ | 1.1 |
| KCl | 2.7 |
| NaCl | 137 |
| NaH₂PO₄ | 0.4 |
| HEPES | 10 |
| D-glucose | 5.6 |
| pH | 6.5 |

TABLE 12
Loading Buffer with Opti-MEM™:

| | Concentration |
|---|---|
| | (mg/mL, except where |
| Component | otherwise indicated) |
| NaCl | 4.38 |
| KCl | 0.36 |
| HEPES | 2.26 |
| NaHCO₃ | 1.01 |
| Dextrose | 0.54 |
| Trehalose | 34.23 |
| Ethanol | 1.00% (v/v) |
| Opti-MEM™ | 24% (v/v) |
| pH | 6.6-6.8 |

siRNA-loading for fresh and stored platelets was evaluated by flow cytometry to obtain a relative quantification of loading efficiency as well as a geometric mean fluorescence intensity (gMFI) of the BLOCK-iT™ Fluorescent Oligo in siRNA-loaded platelets in each buffer condition specified in the figures.

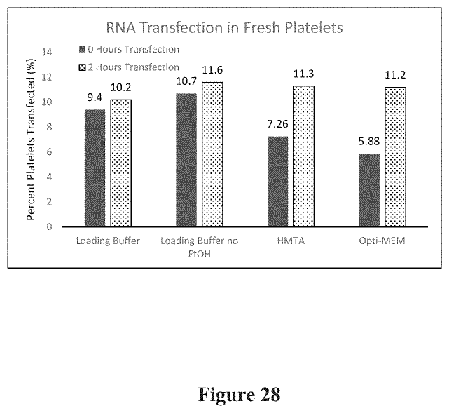

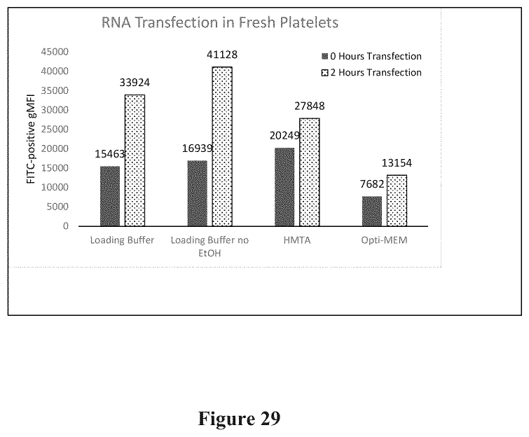

A comparison of fresh platelets incubated for two hours, at room temperature, in different liquid transfection media conditions is depicted in FIG. 28. Transfection efficiency, as a percentage of FITC-positive platelet events by flow cytometry was similar in all conditions at two hours, though an uptake of fluorescent siRNA appeared to occur more rapidly in Loading Buffer (with and without ethanol) than in HMTA or Opti-MEM™. Fluorescence intensity (gMFI) of platelet events positive for fluorescent siRNA was greater in Loading Buffer (with and without ethanol) than when platelets were incubated in HMTA or Opti-MEM™, as shown in FIG. 29. A higher gMFI suggests a greater copy number of fluorescent siRNAs was incorporated into the platelets when the Loading Buffer was used as the liquid transfection medium. N=1.

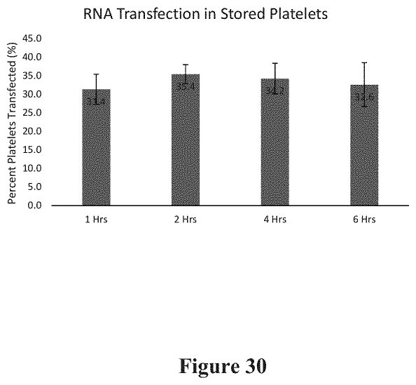

Transfection efficiency for stored platelets was evaluated over the course of six hours by flow cytometry, as shown in FIG. 30. The stored platelets were incubated at room temperature, for the indicated times, in Loading Buffer supplemented with Opti-MEM™ at room temperature. The percentage of platelet events that were positive for fluorescent siRNA reached a maximum at two hours. N=2. Error bars indicate standard deviation (SD). The percentage of fluorescent siRNA-positive platelet events was greater for stored platelets as compared to fresh platelets (see 10.2% shown in FIG. 28 compared to 35.4% shown in FIG. 30). Stored platelets were also incubated in Loading Buffer alone, without Opti-MEM™, at room temperature. The percentage of platelet events that were positive for fluorescent siRNA at two hours was 47.9%, as shown in Table 13 below. Stored platelets incubated in Loading Buffer without Opti-MEM™ had a higher percentage of fluorescent siRNA-positive platelet events as compared to stored platelets incubated in Loading Buffer with Opti-MEM™ (35.4% as shown in FIG. 30 compared to 47.9% shown in Table 13.

TABLE 13

| | Mean percent platelet | Standard Deviation |
|---|---|---|
| Transfection Duration | events transfected (%) | (%) |
| 2 Hours | 47.9 | 4.0 |

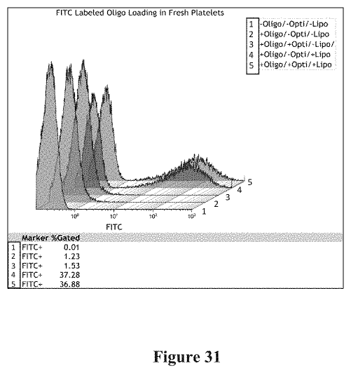

Transfection efficiency of stored platelets was further analyzed in Loading Buffer by maintaining or omitting the Opti-MEM™, fluorescent siRNA, or cationic transfection reagent, as shown in FIG. 31. Stored platelets were incubated at room temperature for one hour under the conditions shown in FIG. 31. The flow cytometer was gated on stored platelets incubated without the Opti-MEM™, fluorescent siRNA, and cationic transfection reagent. Sufficient transfection (as measured by percentage of platelet events that were positive for fluorescent siRNA) was observed when the cationic transfection reagent was present, and this transfection did not appear to be the result of non-specific association of the fluorescent siRNA with the stored platelets. Oligo=BLOCK-iT™ Fluorescent siRNA. Opti=Opti-MEM™. Lipo=Lipofectamine™ RNAiMAX.

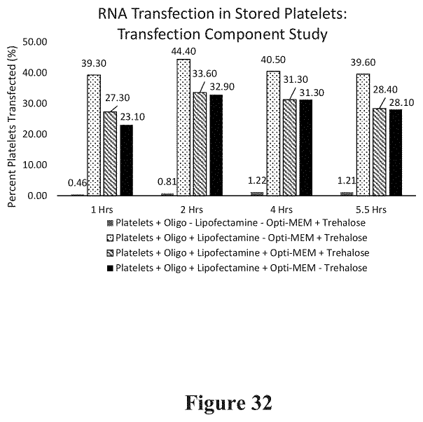

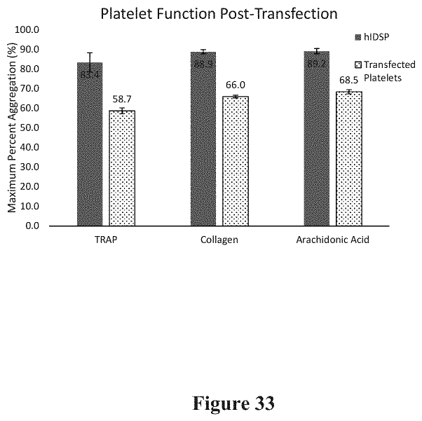

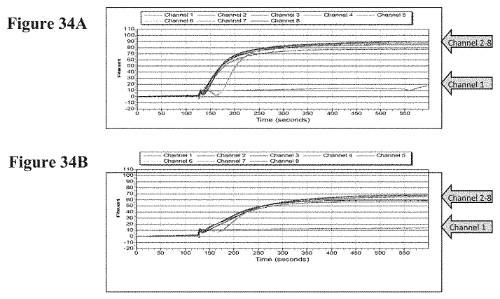

In addition, transfection efficiency of stored platelets was analyzed in Loading Buffer by maintaining or omitting the Opti-MEM™, cationic transfection reagent, or Trehalose, as shown in FIG. 32. The stored platelets were incubated at room temperature for the times and under the conditions shown in FIG. 32. The percentage of platelet events that were positive for fluorescent siRNA appeared to increase when the cationic transfection reagent was maintained and the Opti-MEM™ was omitted. Trehalose did not substantially impact the transfection efficiency. Oligo=BLOCK-iT™ Fluorescent siRNA. Lipofectamine=Lipofectamine™ RNAiMAX.

Protocol 5. Loading Platelets with siRNA

The starting apheresis platelet material was pooled and characterized. The platelet pool was acidified to pH 6.6-6.8 with ACD solution. Platelets were isolated by centrifugation at ˜1470 g for 20 minutes, with slow acceleration and braking. The supernatant plasma was aspirated and disposed.

The cationic transfection reagent (Lipofectamine™ RNAiMAX, 0.24% v/v Final) was diluted in HBS and the pH was adjusted to 6.6-6.8 with hydrochloric acid. The transfection period was 2 hours. The concentration of lipofectamine was 0.002% v/v. The fluorescent siRNA (BLOCK-iT™ Fluorescent siRNA, 0.377 μM Final) was diluted in HBS in a separate container as compared to the cationic transfection reagent. Both the cationic transfection reagent in HBS and fluorescent siRNA in HBS were incubated separately for 5 minutes at room temperature, after which time, they were combined and incubated together for an additional 10 minutes at room temperature. The platelets were resuspended in Loading Buffer and mixed with the combined cationic transfection reagent/fluorescent siRNA in HBS mixture. The platelets were transferred to an air-permeable bag (e.g. FEP bag) and incubated at room temperature (˜22° C.) with gentle agitation (e.g. 20 RPM on an orbital shaker). The resulting siRNA-loaded platelets were then analyzed further, as described herein.

### Example 12. Response to Agonists by siRNA-Loaded Platelets

Multiple platelet aggregation agonists were tested for their effect on siRNA-loaded stored platelets. siRNA-loaded stored platelets were prepared as described in Protocol 5. Platelet aggregation agonists were incubated for 5 minutes at room temperature with the platelets, under gentle agitation, and washed by centrifugation (centrifugation at ˜1470 g for 20 minutes, supernatant aspirated and disposed). The agonists tested were: thrombin receptor activating peptide (TRAP) at 20 μM, collagen at 10 μg/mL, and arachidonic acid at 50 μg/mL.) Light transmission aggregometry was used to evaluate each agonist's effect, as shown in FIG. 6. Although the aggregation response of siRNA-loaded stored platelets was lower as compared to non-loaded stored platelets, siRNA-loaded stored platelets still retained a strong aggregation response to all of the agonists tested. hIDSP=originating stored platelets. Transfected platelets=siRNA-loaded platelets.

Raw aggregometry results, as shown in FIG. 34A and FIG. 34B, indicated siRNA-loaded stored platelets began to aggregate immediately after each agonist was added (˜120 seconds). Channel 1=negative control where stored platelets were incubated in Loading Buffer alone. Channels 2-4=TRAP. Channels 5-6=collagen. Channels 7-8=arachidonic acid.

### Example 13 Agonist Activation Increased Loading of siRNA to Fresh Platelets

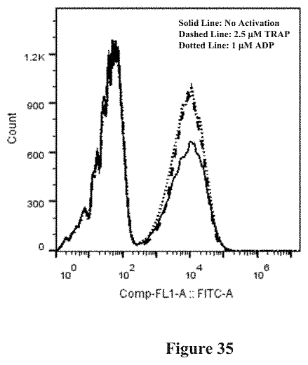

Platelet aggregation agonists were tested for their effect on loading of siRNA to fresh platelets, FIG. 35 and Table 14. Fresh platelets were exposed to adenosine diphosphate (ADP, 1 μM) and/or TRAP (2.5 μM) for 5 minutes followed by siRNA-loading with fluorescent siRNA (BLOCK-iT™ Fluorescent Oligo). Fresh platelets treated with platelet aggregation agonists prior to siRNA-loading demonstrated higher transfection efficiency as compared to an untreated control (52% (ADP) and 48% (TRAP) as compared to 44% (untreated)).

TABLE 14

| Agonist | Mean percent platelet events transfected (%) |
|---|---|
| None | 44 |
| ADP (1 μM) | 52 |
| TRAP (2.5 μM) | 48 |

### Example 14. Retention of siRNA in Thrombosomes Derived from siRNA-Loaded Platelets

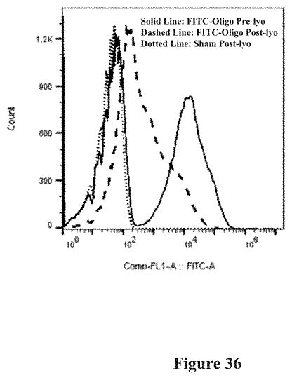

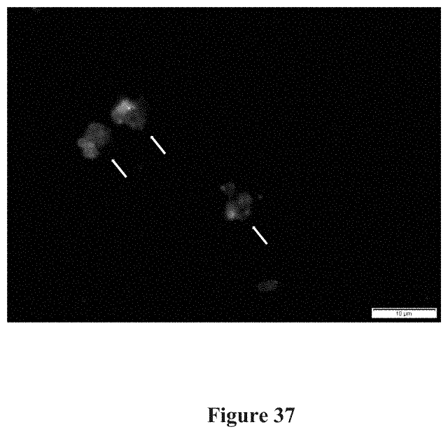

siRNA-loaded stored platelets were prepared, following Protocol 5, and lyophilized to produce Thrombosomes. The Thrombosomes were then rehydrated and analyzed for retention of siRNA, as shown in Table 15 below and FIG. 36.

TABLE 15

| Parameter | Pre-lyophilization | Post-rehydration |
|---|---|---|
| Platelet count (*10⁶/mL) | 235 | 155 |
| Percent platelet events | 52 | 58 |
| transfected (%) | | |
| gMFI of transfected | 1194 | 12394 |
| platelet events | | |

A comparison of pre-lyophilization and post-rehydration siRNA-loaded stored platelets for both percentage of fluorescent siRNA positive platelet events and gMFI of platelet events indicated that a substantial amount of fluorescent siRNA was retained in the rehydrated Thrombosomes. Sham Post-lyo=negative control stored platelets post-lyophilization and rehydration. FITC-Oligo Post-lyo=BLOCK-iT™ Fluorescent siRNA transfected stored platelets post-lyophilization and rehydration. FITC-Oligo Pre-lyo=BLOCK-iT™ Fluorescent siRNA transfected stored platelets pre-lyophilization.

### Example 15. Internalization of siRNA in Thrombosomes

siRNA-loaded stored platelets were prepared according to Protocol 5, and lyophilized to form Thrombosomes. The Thrombosomes were analyzed after rehydration using fluorescence microscopy. FITC signal from the fluorescent siRNA was observed in the cytosol, with some punctate accumulation, as shown in FIG. 37. This signal indicated retention of the internalized fluorescent siRNA in Thrombosomes after rehydration. Arrows indicate individual fluorescent Thrombosomes.

### Example 16. Occlusion of Collagen-Coated Microchannels by Thrombosomes Derived from siRNA-Loaded Platelets

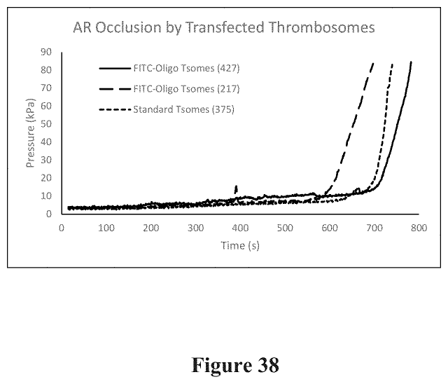

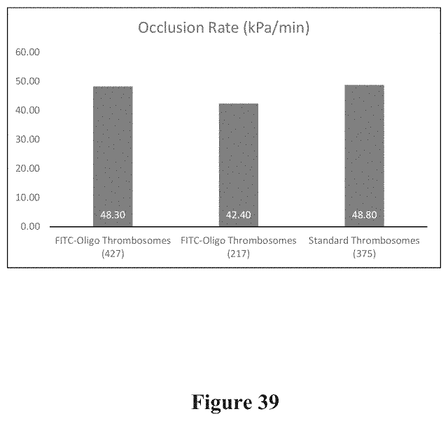

siRNA-loaded platelets were prepared, following Protocol 5, and lyophilized to produce Thrombosomes. The Thrombosomes were then rehydrated and analyzed for occlusion of a collagen-coated microchannel via T-TAS®, as shown in Table 16 below and FIGS. 38 and 39. The occlusion experiment via T-TAS® was conducted following Protocol 6.

siRNA-loaded Thrombosomes were able to produce a rate of occlusion similar to that of non-loaded Thrombosomes.

TABLE 16

| | Thrombosome | | | Rate of | Area |
|---|---|---|---|---|---|
| | Count in GK | | | occlusion | under |
| Sample | PPP (*10³/μL) | T10 | T80 | (kPa/min) | curve |
| siRNA-loaded | 427 | 11:35 | 13:02 | 48.3 | 1457.1 |
| Thrombosomes | | | | | |
| (Run 1) | | | | | |
| siRNA-loaded | 217 | 9:58 | 11:37 | 42.4 | 1566.4 |
| Thrombosomes | | | | | |
| (Run 2) | | | | | |
| Non-loaded | 375 | 10:54 | 12:20 | 48.8 | 1479.8 |
| Thrombosomes | | | | | |

Protocol 6. T-TAS® Occlusion Rate Measurement

The T-TAS® was prepared for use according to the manufacturer's instructions. Briefly, the calcium corn trypsin inhibitor (CaCTI, Diapharma TR0101) and AR chip (containing collagen and tissue factor, Diapharma TC0101) were allowed to warm to room temperature prior to use. Sufficient mineral oil was pumped into the reservoir on the instrument (˜2 mL as each 30-minute run requires ˜400 μL mineral oil). Ethylenediaminetetraacetic acid (EDTA) was loaded into the water/EDTA line of the instrument and was primed for at least 15 seconds to flush any water or bubbles out of the EDTA lines. The dispensed liquid was cleaned from the chip stage with a kimwipe. Finally, a systems check was run to ensure pressure readings were within specification.

A vial of approximately 3×10⁸ Thrombosomes were rehydrated by a 10 minute room temperature incubation in 1.2 mL sterile water. The vial was centrifuged (˜3900 g for 10 minutes) to pellet the Thrombosomes. The Thrombosomes were then resuspended in George King (GK) pooled normal human plasma to a concentration of ˜300,000 Thrombosomes/μL. The final Thrombosome concentration was confirmed by a hematology analyzer (AcT diff2, Beckman Coulter). Thrombosomes in GK plasma (480 μL) were then mixed with 20 μL CaCTI by gentle pipetting.

The AR chip was docked onto the T-TAS® stage and a waste reservoir was mounted onto the instrument. The sample injector was prepared by carefully adding (so as not to introduce bubbles) ˜450 μL of the Thrombosome/GK plasma/CaCTI mixture. The T-TAS® instrument was run and data were recorded according to manufacturer's instructions.

### Example 17. Platelet Transfection with miRNA

Methods

Transfection was conducted in a manner analogous to that used for siRNA in Protocol 5, except that the initial transfection period was 30 minutes instead of 2 hours. Two groups, Test Group A (also referred to as Subset A or as “A”) and Test Group B (also referred to as Subset B or as “B”) were tested. In group A, the concentration of lipofectamine was 0.002% v/v. In group B, the concentration of lipofectamine was 0.004% v/v. The starting platelet concentration was of about ˜58,000/μL for both “A” and “B”.

Results

After a 30 minute transfection period, no difference in platelet concentration was observed between “A” and “B” (the platelet concentration was ˜58,000/μL in both cases). After a two hour transfection period, an 18% reduction in platelet count was observed for “B” and only a 3% reduction in platelet count was observed for “A.” Thus, for short (30 minutes or less) transfection times the platelet count is not substantially affected by an increase in lipofectamine concentration in the transfection mixture.

All Counts *10³/μL by AcT diff 2

| | | | % reduction in |
|---|---|---|---|
| | | | platelet count from |
| | 30 Minutes | 120 Minutes | 30 to 120 minutes |
| Group A | 59 | 57 | 3% |
| Group B | 58 | 47 | 18% |

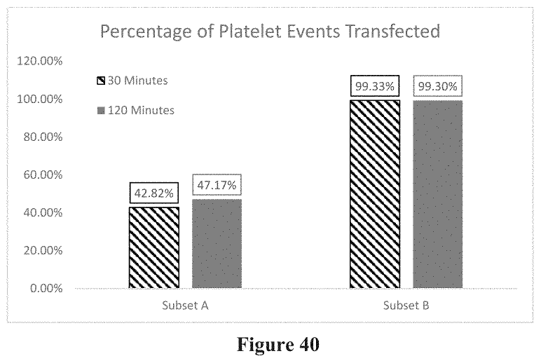

As shown in FIG. 40, in “A”, only ˜43% of platelets contain transfected miRNA after 30 minutes, compared to >99% of platelets in “B.” These values do not change substantially in either group after 2 hours transfection (after 2 hours the percentage of transfected platelets in “A” is 47%, while in “B” it is >99%)—see FIG. 40. This suggests that most of the loading occurs within 30 minutes of exposure to the transfection components.

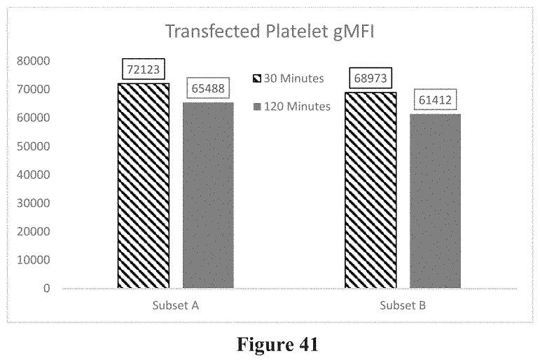

Fluorescence intensity of the transfected platelets was nearly identical for “A” and “B” after 30 minutes (˜72,000 and ˜69,000 gMFI for “A” and “B,” respectively, as shown in FIG. 41), suggesting that a similar amount of miRNA is loaded into each platelet for both “A” and “B.” Decreases in fluorescence intensity over time (e.g., after 2 hours, as shown in FIG. 41) are likely due in part to FITC photobleaching.

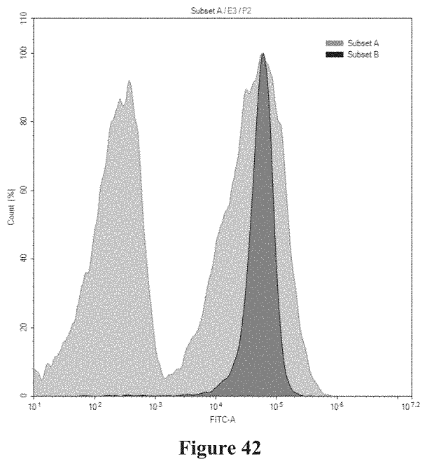

FIG. 42 is a normalized histogram of fluorescence intensity in FITC for all single platelet events collected for “A” (gray) and “B” (black). The bimodal distribution for “A”, in contrast with the unimodal distribution for “B”, indicates a more consistent loading of RNA material in each platelet in “B.” As used herein, “single platelet events” are particles that have been detected by the cytometer, curated to include only platelets and only singlets (that is, excluding two platelets detected simultaneously).

The above results are reproducible across a range of platelet concentrations from about 50,000 platelets/μL to about 100,000 platelets/μL. The above results are reproducible for apheresis “stored” platelets, random donor “stored” platelets (prepared from stored whole blood by fractional centrifugation), and fresh drawn platelets.

### Example 18. Cryopreservation of Platelets Transfected with miR-34a

miR-34a is an anti-oncogenic miRNA found in healthy tissues that downregulates >30 oncogenes and is deficient in many cancers. In this experiment miR-34a with an Alexa Fluor 647 conjugate was used in order to facilitate fluorescent detection.

Platelets were transfected at ˜76,000 platelets/uL (concentration after dilution with transfection components) using 30 nM miR-34a Alexa Fluor 647 and 0.004% (v/v) Lipofectamine RNAiMAX, using the procedure described in Example 17 and a transfection time of 30 minutes.

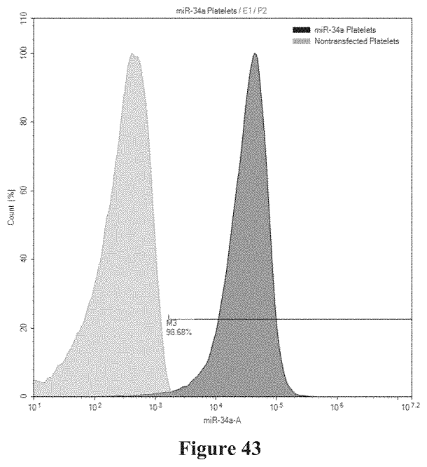

After 30 minutes exposure of the platelets to the miRNA and Lipofectamine complex, platelets were washed by centrifugation (1470 g×20 minutes) and resuspended in the loading buffer of Table 9 to a concentration of approximately 1,200 platelets/uL. Transfected platelets were evaluated by flow cytometry and showed >98% platelets transfected with the fluorescently labeled miR-34a, as shown in FIG. 43.

Transfected platelets were cryopreserved by supplementing with 6% (w/v) polysucrose 400 and 1% (v/v) DMSO. The platelet suspension was divided into 1 mL aliquots in 1.5 mL cryogenic vials, and rapidly frozen in a −80° C. freezer. Storage was maintained at −80° C. until analysis for a minimum of 3 days.

The transfected cryopreserved platelets were then thawed for approximately 5 minutes in a 37° C. water bath prior to analysis by AcT count, flow cytometry, and T-TAS.

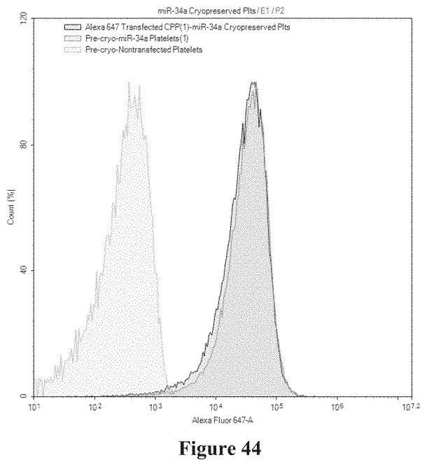

The transfected cryopreserved platelets retained 90% of the fluorescence of the pre-cryopreservation platelet material as evaluated by flow cytometry, as shown in FIG. 44.

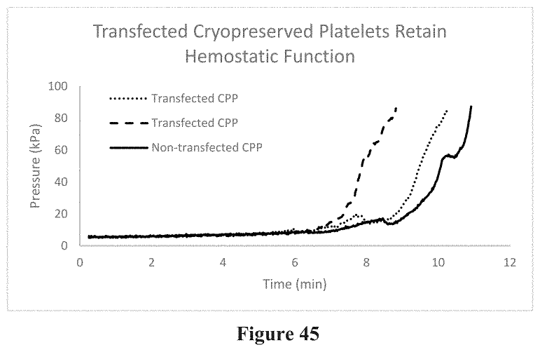

Transfected (thawed, cryopreserved platelets) were prepared for T-TAS analysis by centrifugation and resuspension of the platelet pellet in citrated platelet-poor plasma to a concentration of approximately 300,000 platelets/uL. The cryopreserved platelets in plasma (480 uL) were supplemented with 20 uL CaCTI and flowed across a collagen and tissue factor coated capillary channel under high shear. Clotting and channel occlusion were assessed as a function of flow pressure over time. The results demonstrate that transfected-thawed, cryopreserved, transfected platelets (denoted “transfected CPP” in FIG. 45, which shows plots of flow pressure vs. time in a channel occlusion test for non-transfected platelets and transfected thrombosomes)—retain hemostatic function by rapidly occluding the collagen and tissue factor coated capillary.

### Example 19. Permeabilized Platelets

Examples of drugs and of loading agents are as follows:

| | | Osmotic hypertonic/ |
|---|---|---|
| Endocytosis | Cell penetrating peptide | hypotonic loading |
| BODIPY-vancomycin | BODIPY-vancomycin | BODIPY- |
| | | vancomycin; |
| BODIPY-vancomycin | Dextran 10K | Dextran 10K |
| and ristocetin | | |
| Dextran 10K | Dextran 3K | Dextran 3K |
| Dextran 500K | Dextran 500K | Dextran 500K |
| FITC-Albumin | FITC-albumin | FITC-albumin |
| FITC-Bovine IGG | FITC-Bovine IGG | FMLP |
| FITC-F(ab)₂ | FITC-F(ab)₂ | Histone H1 |
| Histone H1 | FMLP | Lucifer Yellow |
| Lucifer yellow-slow | Histone H1 | PE |
| uptake | | (PHYCOERYTRIN) |
| PE | Lucifer yellow | Rabbit IGG |
| Rabbit IGG | PE | Soybean Trypsin |
| | | Inhibitor |
| Soybean Trypsin | Rabbit IGG | |
| Inhibitor | | |
| | Soybean Trypsin | |
| | Inhibitor | |

The table below provides examples of various types of drugs that may be loaded into the platelets, such as antibiotics like vancomycin and ristocetin; antibodies such as rabbit IGG; antibody fragments such as F(ab)₂; peptides such as FMLP; protease inhibitors such as Soybean Trypsin Inhibitor; and Lucifer yellow. Similarly, the table below provides examples of various types of loading agents, such as albumin or such as dextrans having various molecular weights.

More particular examples are as follows:

| | | Osmotic hypertonic/ |
|---|---|---|
| Endocytosis | Cell penetrating peptide | hypotonic loading |
| BODIPY-vancomycin | BODIPY-vancomycin | BODIPY- |
| | | vancomycin; |
| FITC-Bovine IGG | Dextran 500K | Dextran 10K |
| Rabbit IGG | FITC-albumin | Dextran 3K |
| | FITC-Bovine | FMLP |
| | FMLP | |
| | Rabbit IGG | |
| | Soybean Trypsin | |
| | Inhibitor | |

In some embodiments the drug is vancomycin. In some embodiments where the drug is vancomycin, the drug is labeled with BODIPY (boron-dipyrromethene or 4,4-difluoro-4-bora-3a,4a-diaza-s-indacene). In some embodiments, the loading step comprises the use of dextran as a lyophilizing agent. In some embodiments the drug is an antibody. In some embodiments when the drug is an antibody, the drug is labeled with FITC (fluorescein isothiocyanate or 3′,6′-dihydroxy-6-isothiocyanatospiro[2-benzofuran-3,9′-xanthene]-1-one). In some embodiments, the drug is soybean trypsin inhibitor.

In some embodiments, drug-loaded platelets, drug-loaded platelet derivatives, or drug-loaded thrombosomes may shield the drug from exposure in circulation, thereby reducing or eliminating systemic toxicity (e.g. cardiotoxicity) associated with the drug. In some embodiments, drug-loaded platelets, drug-loaded platelet derivatives, or drug-loaded thrombosomes may also protect the drug from metabolic degradation or inactivation. In some embodiments, drug-loaded platelets, drug-loaded platelet derivatives, or drug-loaded thrombosomes may be used in any therapeutic setting in which expedited healing process is required or advantageous.

In some embodiments, provided herein is a method of treating a disease as disclosed herein, comprising administering drug-loaded platelets, drug-loaded platelet derivatives, or drug-loaded thrombosomes as disclosed herein. In some embodiments, provided herein is a method of treating a disease as disclosed herein, comprising administering cold stored, room temperature stored, cryopreserved thawed, rehydrated, and/or lyophilized platelets, platelet derivatives, or thrombosomes as disclosed herein. In some embodiments, the disease is, Traumatic Brain injury. In some embodiments, the disease is, ITP. In some embodiments, the disease is TTP.

Examples of diseases (therapeutic indications) that may be treated with the drug-loaded platelets are as follows:

Therapeutic indications
Colitis
Corynebacterium
Enterococci
Methicillin-resistant Staphylococci aureus
Streptococcus pneumoniae
Viridans streptococci
Staphylococcal infection
Chronic inflammatory demyelinating polyneuropathy
Guillain-Barre syndrome
Immune Thrombocytopenia
Kawasaki disease
Lupus
Multiple Sclerosis
Myasthenia gravis
Myositis
Thrombosis
Crohn's disease
Macular degeneration
Albuminaemia
Burns (>30% body surface area, after first 24 hours)
Cardiac surgery (last line of treatment)
Cirrhosis with refractory ascites
Haemorrhagic shock (when patient not responsive to
crystalloids/colloids)
Hepatorenal syndrome (used in combination with
vasoconstrictive drugs)
Nephrotic syndrome (for patient with albumin
<2 g/dL with hypovolaemia and/or pulmonary edema)
Organ transplantation
Paracentesis
Spontaneous bacterial peritonitis (in addition with
antibiotics)
Hypovolemia
Iron deficiency (intramuscular injection of ⁵⁹Fe iron
dextran, INFeD. For patients who do not respond to
or unable to take oral iron supplement)
Fluid resuscitation in shock
Aneurysms
Artherosclerosis
Cardiovascular diseases (post-myocardial infarction
remodeling, cardiac regeneration, cardiac fibrosis,
viral myocarditis, cardiac hypertrophy, pathological
cardiac remodeling)
Genetic disorders
Infectious diseases
Metabolic diseases
Opthalmic conditions (retinal angiogenesis)
Pulmonary hypertension

| Cargo | Therapeutic indications |
|---|---|
| Vancomycin | Colitis |
| (antibiotic) | Corynebacterium |
| | Enterococci |
| | Methicillin-resistant Staphylococci aureus |
| | Streptococcus pneumoniae |
| | Viridans streptococci |
| Vancomycin- | Staphylococcal infection |
| Ristocetin | |
| (antibiotic) | |
| IgG (antibody) | Chronic inflammatory demyelinating polyneuropathy |
| | Guillain-Barre syndrome |
| | Immune Thrombocytopenia |
| | Kawasaki disease |
| | Lupus |
| | Multiple Sclerosis |
| | Myasthenia gravis |
| | Myositis |
| Fab'2 (fragment | Abciximab (Reopro) used for clot prevention in |
| of antibody) | angioplasty |
| | Certolizumab pegol (Cimzia) treat moderate to |
| | severe Crohn's disease |
| | Ranibizumab (Lucentis) treat macular degeneration |
| Albumin | Albuminaemia |
| (carrier protein) | Burns (>30% body surface area, after first 24 hours) |
| | Cardiac surgery (last line of treatment) |
| | Cirrhosis with refractory ascites |
| | Haemorrhagic shock (when patient not responsive to |
| | crystalloids/colloids) |
| | Hepatorenal syndrome (used in combination with |
| | vasoconstrictive drugs) |
| | Nephrotic syndrome (for patient with albumin |
| | <2 g/dL with hypovolaemia and/or pulmonary edema) |
| | Organ transplantation |
| | Paracentesis |
| | Spontaneous bacterial peritonitis (in addition with |
| | antibiotics) |
| Dextran | Hypovolemia |
| | Iron deficiency (intramuscular injection of ⁵⁹Fe iron |
| | dextran, INFeD. For patients who do not respond to |
| | or unable to take oral iron supplement) |
| | Fluid resuscitation in shock |

In some embodiments, a drug may be fluorescent or labeled with a fluorescent moiety. For such a fluorescent or labeled drug, a correlation may be established between the fluorescence intensity and its concentration, and such a correlation may then be used to determine the concentration of the drug over a range of values.

Examples of loading buffer that may be used are shown in Tables 17-20:

TABLE 17
Loading Buffer

| | Concentration (mM |
|---|---|
| | unless otherwise |
| Component | specified) |
| NaCl | 75.0 |
| KCl | 4.8 |
| HEPES | 9.5 |
| NaHCO3 | 12.0 |
| Dextrose | 3 |
| Trehalose | 100 |
| Ethanol | 1% (v/v) |

Table 17. Loading Buffer is used to load platelets via endocytosis at 37° C. with gentle agitation as sample is placed on a rocker. Adjust pH to 6.6-6.8

TABLE 18
Buffer A

| | Concentration (mM |
|---|---|
| | unless specified |
| Component | otherwise) |
| CaCl₂ | 1.8 |
| MgCl₂ | 1.1 |
| KCl | 2.7 |
| NaCl | 137 |
| NaH₂PO₄ | 0.4 |
| HEPES | 10 |
| D-glucose | 5.6 |
| pH | 6.5 |

Table 18. Buffer A is used for loading platelets with liposome encapsulated drug. Incubation done at 37° C. with gentle agitation as sample is placed on a rocker.

TABLE 19
Buffer B

| | Concentration (mM |
|---|---|
| | unless otherwise |
| Component | specified) |
| Buffer and Salts | Table 4 (below) |
| BSA | 0.35% |
| Dextrose | 5 |
| pH | 7.4 |

Table 19. Buffer B is used when incubating platelets with fluorophore conjugated antibodies for flow cytometry.
This incubation is done at room temperature in the dark.
Albumin is an optional component of Buffer B

TABLE 20
Concentration of HEPES and of Salts in Buffer B

| | Concentration (mM |
|---|---|
| | unless otherwise |
| Component | specified) |
| HEPES | 25 |
| NaCl | 119 |
| KCl | 5 |
| CaCl₂ | 2 |
| MgCl₂ | 2 |
| glucose | 6 g/l |

In Table 20 the pH adjusted to 7.4 with NaOH

Albumin is an optional component of Buffer B

In some embodiments, drug-loaded platelets are prepared by incubating the platelets with the drug in a loading buffer having the components shown in the table below.

In some embodiments, the loading buffer has the components as listed above in Table 17. In some embodiments, incubation is performed at 37° C. using a platelet to drug volume ratio of 1:2, using different incubation periods.

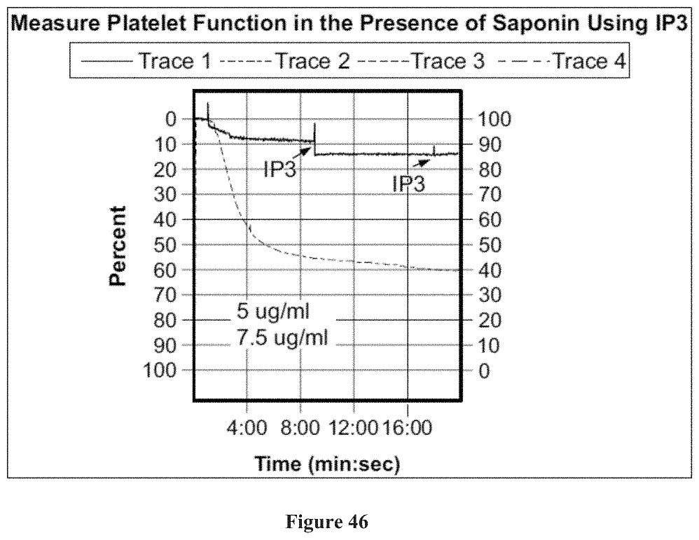

In FIG. 46, platelets were incubated in the presence of saponin at a concentration of either 5 μg/mL or 7.5 μg/mL to permeabilize platelet cell membranes. 1, 4, 5-triphosphate (IP3) was added at the time points indicated. Saponin-mediated permeabilization of platelets was measured using standard light transmission aggregometry (LTA). The data of FIG. 46 show that saponin permeabilizes platelet cell membranes in a concentration-dependent manner.

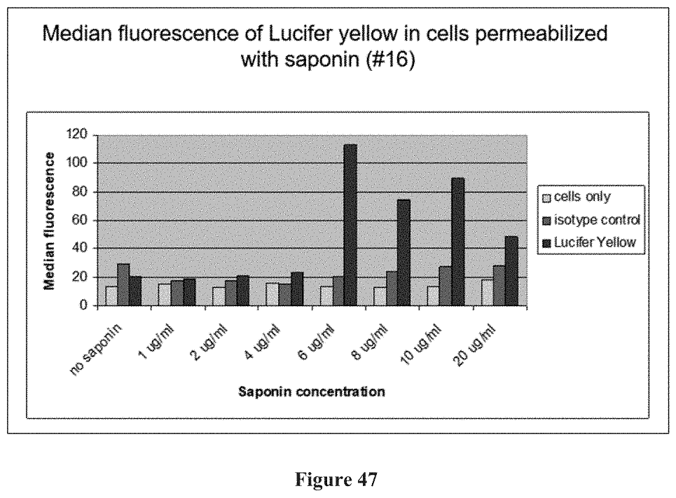

Saponin permeabilizes platelet cell membranes in a concentration dependent manner. Platelets were incubated in the presence of saponin at a concentration of either 5 μg/mL or 7.5 μg/mL to permeabilize platelet cell membranes. 1, 4, 5-triphosphate (IP3) was added at the time points indicated. Saponin-mediated permeabilization of platelets was measured using standard light transmission aggregometry (LTA) (FIG. 46). Platelets were incubated with increasing concentrations of a detergent (e.g., saponin) and using Hepes modified Tyrode's albumin (HMTA). Cells only bars represent samples in which the platelets were incubated in the absence of Lucifer yellow. Isotype control bars represent samples in which the platelets were incubated in the presence of a reference antibody. Lucifer Yellow bars represent samples in which the platelets were incubated in the presence of Lucifer yellow. In FIG. 47, platelets were incubated in the presence of saponin at the concentrations indicated on the x-axis using Hepes modified Tyrode's albumin (HMTA). Cells only bars represent samples in which the platelets were incubated in the absence of Lucifer yellow. Isotype control bars represent samples in which the platelets were incubated in the presence of a reference antibody. Lucifer Yellow bars represent samples in which the platelets were incubated in the presence of Lucifer yellow. Fluorescence was measured using flow cytometry. These data show that saponin increases permeabilization of platelet cell membranes in a concentration-dependent manner. FIG. 47 shows the median fluorescence as measured by flow cytometry. The results show a saponin concentration of about 6 μg/ml to about 10 μg/ml resulting in uptake of Lucifer Yellow by the permeabilized platelets.

### Example 20. Drug-Loaded Platelets

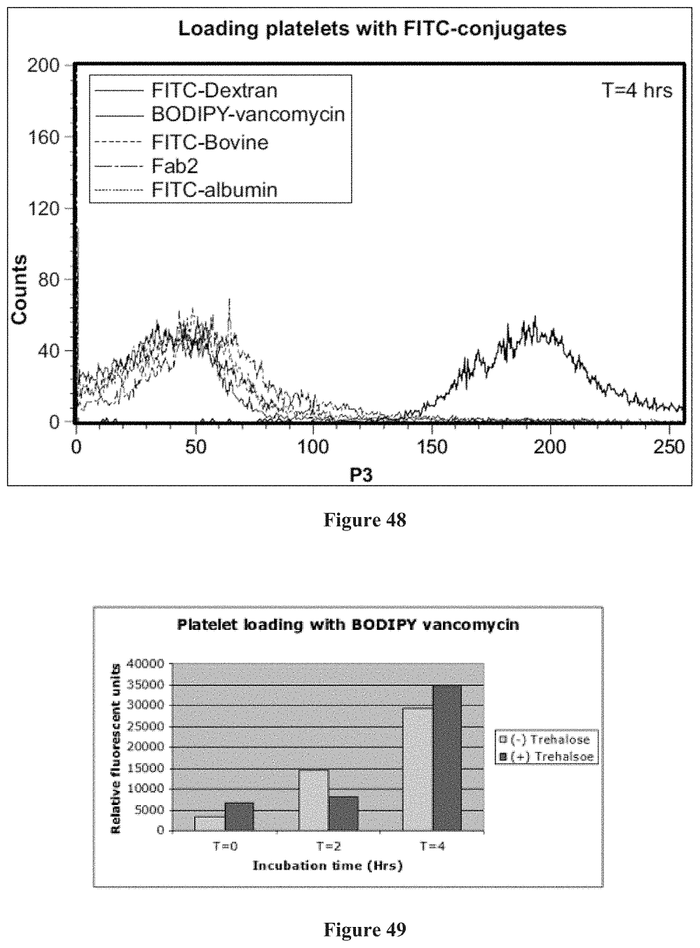

Platelets were incubated with BODIPY tagged vancomycin in increasing time intervals (0, 2, and 4 hours). The experiment was performed in duplicate with and without the presence of trehalose. In FIG. 49, platelets were separately incubated with fluorescently-labeled BODIPY-vancomycin for zero, two, or four hours in the presence or absence of trehalose. The fluorescently-labeled BODIPY-vancomycin was loaded into platelets via fluid phase endocytosis. Flow cytometry was performed to determine endocytotic loading efficiency. These data show that fluorescently-labeled vancomycin was more efficiently loaded into platelets via fluid phase endocytosis at four hours in the presence of trehalose than in the absence of trehalose. FIG. 49 shows flow cytometry data indicating endocytic loading efficiency of the fluorescently-labeled BODIPY-vancomycin via fluid phase endocytosis. These data show that fluorescently-labeled vancomycin was more efficiently loaded into platelets via fluid phase endocytosis at four hours in the presence of trehalose than in the absence of trehalose. In FIG. 48, platelets were separately incubated for four hours with fluorescently-labeled: BODIPY-vancomycin, FITC-bovine IgG, FITC-Fab2, FITC-albumin, or FITC dextran. The fluorescently-labeled molecules were loaded into platelets via fluid phase endocytosis. Flow cytometry was performed to determine endocytotic loading efficiency. These data show that fluorescently-labeled vancomycin was efficiently loaded into platelets via fluid phase endocytosis. FIG. 48 also shows flow cytometry data providing endocytic loading efficiency of other fluorescent conjugates including BODIPY-vancomycin, FITC-bovine IgG, Fab2, and FITC-albumin in platelets.

### Example 21. Loading Platelets with Liposome Encapsulated Vancomycin

The starting apheresis platelet material can be pooled and characterized. The platelet pool can be acidified to pH 6.6-6.8 using Acid Citrate Dextrose solution. Platelets can be isolated by centrifugation at ˜1500 g for 20 minutes, with slow acceleration and braking. The supernatant plasma can be aspirated and disposed of.

The platelets can be suspended in Buffer A at a concentration of 200,000 platelets/μl. The components of Buffer A are shown above Table 18.

While the platelets can be centrifuged, liposome-encapsulated vancomycin can be prepared as follows: lyophilized phospholipids (Sigma-Aldrich, SKU #DOX-1000) can be rehydrated with a 2 mg/ml solution of Vancomycin in PBS; the rehydrated mixture can be subjected it to a vortex for 30 seconds and can be incubated at 37° C. for 30 minutes.

The platelets in Tyrode's HEPES buffer and the liposomal Vancomycin can be mixed and the mixture can be incubated at 37° C. for 30 minutes.

The resulting Vancomycin-loaded platelets can be washed in 1 mL Tyrode's HEPES buffer to remove unincorporated liposome by centrifugation at 1500 g for 20 minutes.

Optionally, the lyophilized Vancomycin-loaded platelets can be suspended in water at a concentration suitable for the uses disclosed herein.

### Example 22. Hyper/Hypotonic Platelet Loading

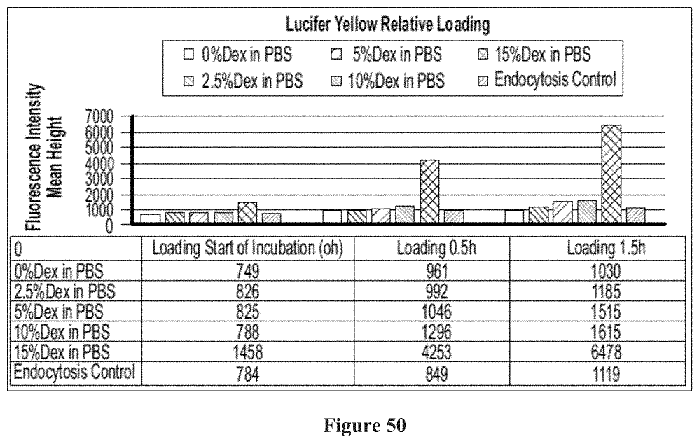

Platelets were isolated by centrifugation at 1000 g for 10 minutes. The platelets were then resuspended in a hypertonic pre-treatment solution composed of dextrose at 2.5%, 5%, 10%, and 15% dextrose in PBS at pH 6.5 (w/v). Platelets were then incubated at room temperature for 30 minutes. Following incubation the platelets were again isolated via centrifugation at 1000 g for 10 minutes. The supernatant was aspirated and the platelets were resuspended in loading buffer and incubated at room temperature for 30-60 minutes. Experiments with this method demonstrated significant loading of the fluorescent dye Lucifer yellow for platelets incubated with high osmolarity pre-treatment solutions (FIG. 50). Fluorescence measurements were taken at 0 hours, 0.5 hours, and 1.5 hours. Loading was generally correlated with the osmotic gradient derived from the difference in osmolarity between the pre-treatment solution and the loading solution. 15% dextrose in PBS demonstrated substantially higher loading.

### Example 23. Hypertonic/Hypotonic Loaded Platelet Morphology and Viability

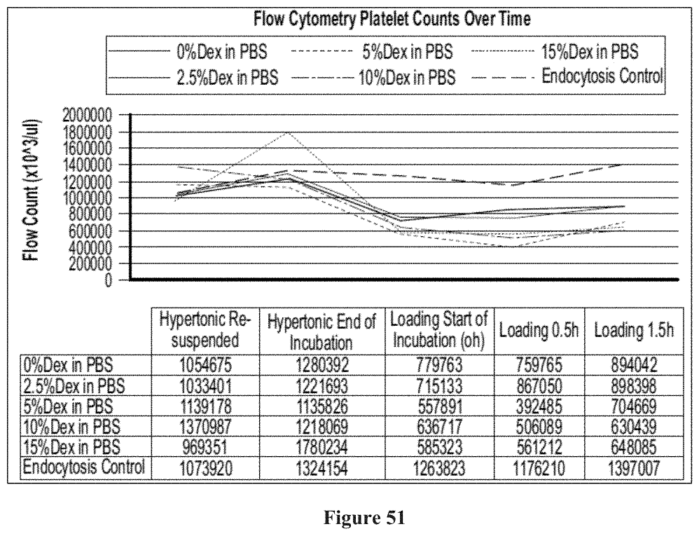

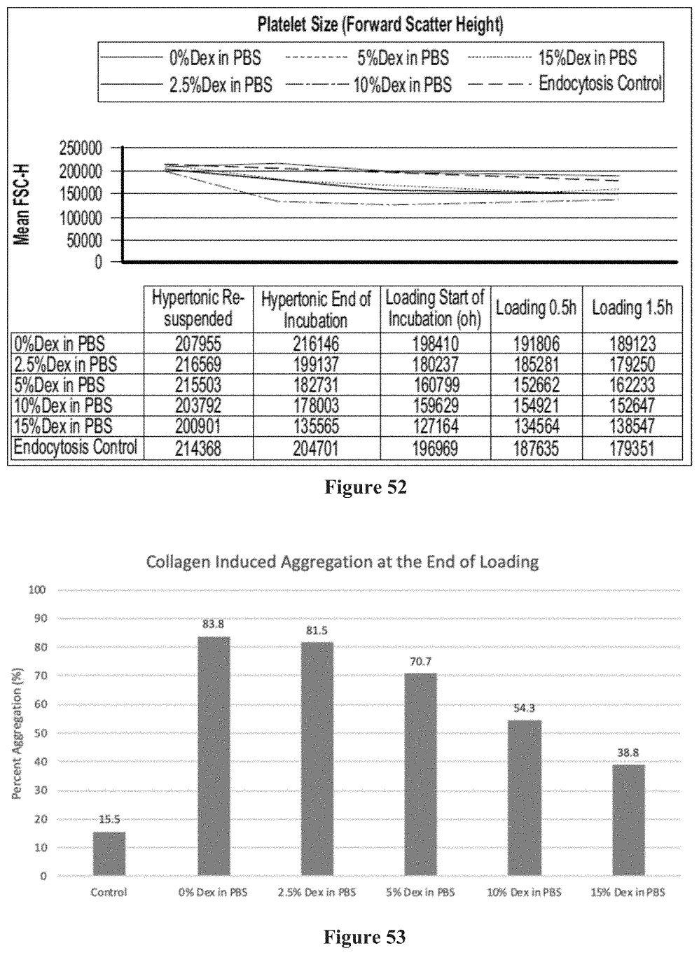

Platelet morphology and viability was assessed for each condition with flow cytometry platelet counts (FIG. 51) and flow cytometry measurements of cell size (FIG. 52). Platelets across all conditions maintained relatively stable counts and acceptable cell sizes. Additionally, platelets were tested for functionality at the end of the process (after 1.5 hours of loading) by aggregometry. Platelet aggregation in response to collagen was measured for platelets from each condition. The more hypertonic pre-treatment solutions gave lower aggregation responses, however, the responses were still above the baseline (FIG. 53).

### Example 24. Electroporation Loaded Platelets

Phalloidin:

Apheresis platelets were prepared and washed in Loading Buffer (Table 17) to a target concentration of 1,500 k/μl by centrifugation.

Phalloidin-CF488A was added to the prepared platelets to a final concentration of 5 U/mL to the platelet suspension.

Aliquots of the washed platelet suspension and peptide suspension (Phalloidin-CF488A) were electroporated under the following parameters: 2000 V, 0.2 ms, 0.4 cm cuvette width, and exponential waveform. Electroporation was performed with Gene Pulser Xcell Electroporation Systems (Biorad).

The electroporated sample was transferred to a polypropylene microcentrifuge tube and allowed to rest in the dark at room temperature for 30 minutes. The microcentrifuge containing the electroporated sample was inverted by hand every 5 to 10 minutes to prevent sedimentation and to promote mixing. The loaded platelets can be washed and used directly for desired applications. Platelets loaded with Phalloidin-CF® 488A (Biotium, Cat. #00042) were evaluated by flow cytometry for FITC signal post-electroporation.

Variations to this protocol can be made. For example, fresh platelets can also be used and the target concentration of platelets (e.g., fresh platelets, apheresis platelets) can be between about 500,000/μl and about 2,500,000/μl. Also, the electroporation voltage can be varied between 500 V and 3,000 V. The cuvette width may be selected from 0.1 cm, 0.2 cm, or 0.4 cm. Electric pulse waveform may be selected from square waveform or exponential waveform. Pulse duration may be varied between 0.05 ms and 2.0 ms, and the total number of exposures to the electric field may be increased.

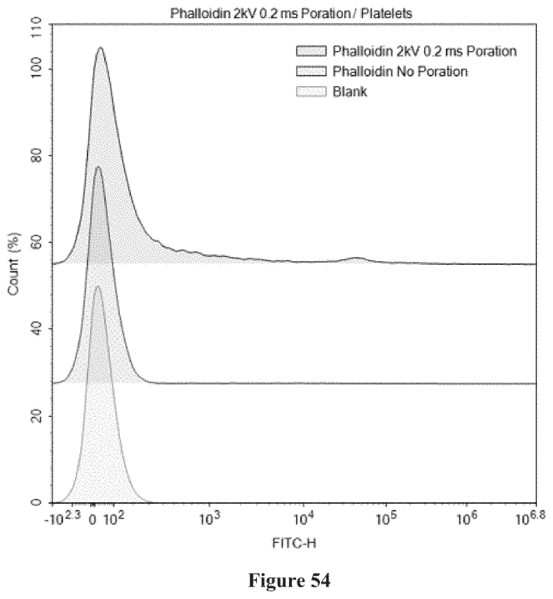

FIG. 54 shows that platelets, without electroporation, that are loaded with Phalloidin-CF® 488A (5 U/mL); “Phalloidin no poration”) are indistinguishable from platelets not loaded with Phalloidin-CF® 488A (“Blank”). FIG. 54 also shows that Phalloidin-CF® 488A loading into platelets is not uniform under electroporation conditions at 2000 V, 0.2 ms, exponential waveform in a 0.4 cm cuvette width. Without wishing to be bound by any theory, the non-uniform loading may be the result of the population of cells that may be in different actin polymerization states. Among the electroporated platelets a wide distribution of fluorescence intensity is observed. In total, about 20% of platelets have detectable Phalloidin-CF® 488A.

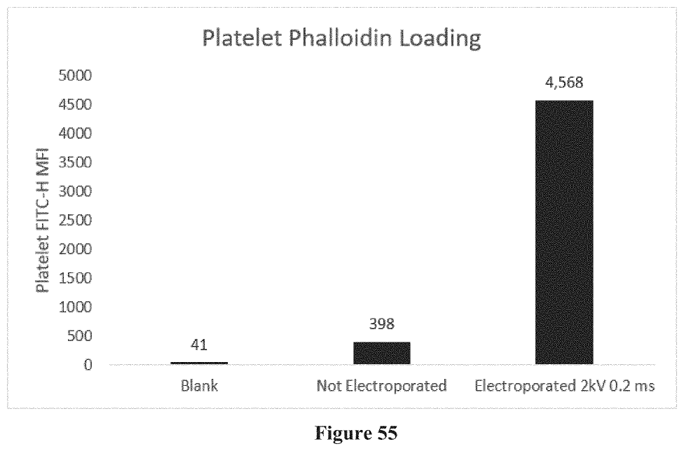

FIG. 55 shows mean platelet FITC-H intensity by flow cytometry for samples with or without 5 U/mL phalloidin-CF® 488A, and with or without electroporation (2000 V, 0.2 ms, exponential waveform in a 0.4 cm cuvette width). Background fluorescence signal is low for both the sample containing no phalloidin-CF® 488A “Blank” and the sample containing Phalloidin-CF® 488A in the absence of electroporation (“Not Electroporated”). Electroporation in the presence of Phalloidin-CF® 488A (“Electroporated 2 kV 0.2 ms”), the mean FITC-H intensity of the entire platelet population increases more than 10-fold over background.

The results show that Phalloidin-CF® 488A binds filamentous actin and is typically used for high-resolution microscopy. Also, the Phalloidin-CF® 488A accumulates in electroporated cells that also have filamentous actin. The results demonstrate that a peptide which is typically membrane impermeable (e.g. Phalloidin) can be introduced in a selective manner to live platelets.

Streptavidin

Platelets were prepared by the protocol described in this example. The protein used below was Streptavidin-Dylight 488 (ThermoFisher Cat. #21832) at a concentration of 150 μg/mL. Platelets loaded with Streptavidin-Dylight 488 were evaluated by flow cytometry for FITC signal post-electroporation. Streptavidin is typically used for immunostaining and signal amplification in combination with biotin-conjugated antibodies. Streptavidin is not expected to bind specifically to any platelet component.

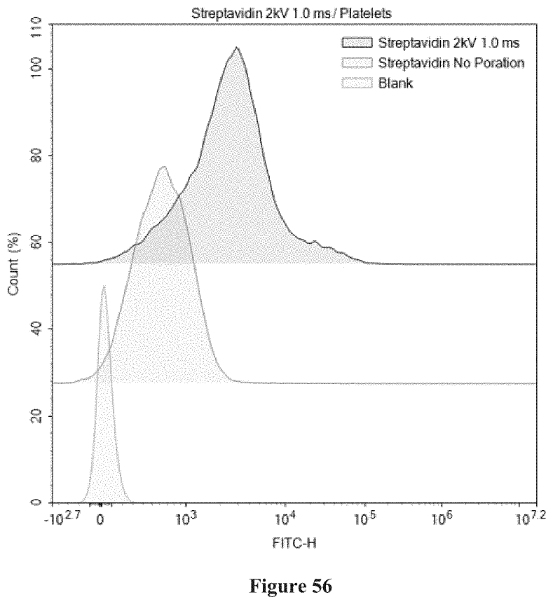

FIG. 56 is a histogram of platelet+/−Streptavidin-Dylight 488 FITC-H intensity on a biexponential scale. With no electroporation, Platelets loaded with Streptavidin-Dylight 488 (150 μg/mL; “Streptavidin no Poration”) that are not exposed to electroporation have slightly elevated fluorescence background in FITC-H compared to platelets not loaded with Streptavidin-Dylight 488 (“Blank”). Platelets loaded with Streptavidin-Dylight 488 exposed to electroporation (2000 V, 1.0 ms, exponential waveform in a 0.4 cm cuvette width), saw fairly uniform loading, as evidenced by the unimodal platelet population distribution. Among electroporated Streptavidin-Dylight 488 FITC-H loaded platelets over 50% have fluorescence signal over background.

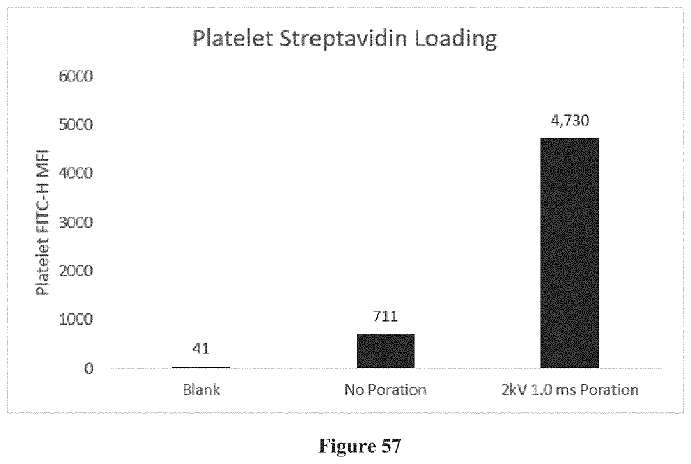

FIG. 57 shows mean platelet FITC-H intensity by flow cytometry for samples with or without 150 μg/mL of Streptavidin-Dylight 488 FITC-H, and with or without electroporation (2000 V, 1.0 ms, exponential waveform in a 0.4 cm cuvette width). Background fluorescence signal is low for the sample containing no Streptavidin-Dylight 488 FITC-H (“Blank”) and is moderately elevated for the sample containing Streptavidin-Dylight 488 FITC-H but absent electroporation (“Not Electroporated”). After electroporation in the presence of Streptavidin-Dylight 488 (“Electroporated 2 kV 1.0 ms”), the mean FITC-H intensity of the entire platelet population increases more than 6 fold over the background.

Lucifer Yellow

Platelets were prepared by the protocol described in this example. The small molecule used was Lucifer Yellow at a concentration of 2.5 mM. Platelets loaded with Lucifer Yellow were evaluated by flow cytometry for AmCyan signal post-electroporation. Lucifer Yellow is a small, membrane impermeable hydrophilic fluorescent molecule that is generally used as a means for measuring endocytosis turnover, and is not expected to bind any platelet-specific components. Platelets were loaded with Lucifer Yellow by electroporation (1400 V, 0.2 ms, 0.4 cm cuvette width, exponential waveform) with one, two, or three exposures to the electric field. Samples were allowed to rest 30 minutes in microcentrifuge tubes between repeated exposures.

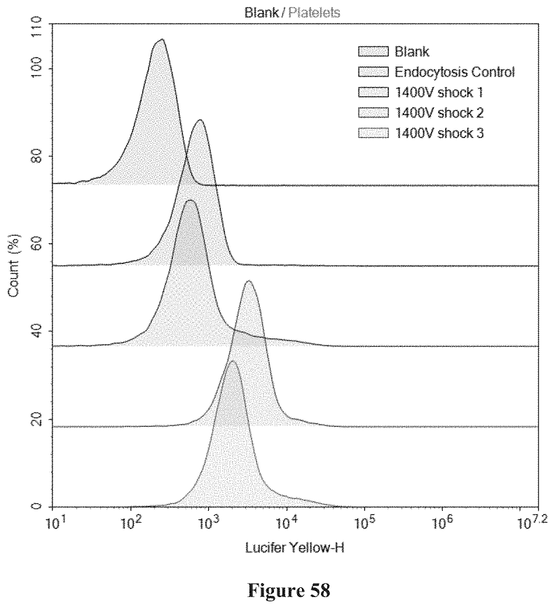

FIG. 58 is a histogram showing Lucifer Yellow fluorescence in platelets under various loading conditions. Platelets exposed to one shock at 1400 V had similar Lucifer Yellow loading compared to a platelet sample that endocytosed Lucifer Yellow for three hours in Loading Buffer (Table 17). Maximum Lucifer Yellow fluorophore loading was seen after two shocks at 1400 V, and there was no benefit to additional electroporation cycles after two shocks.

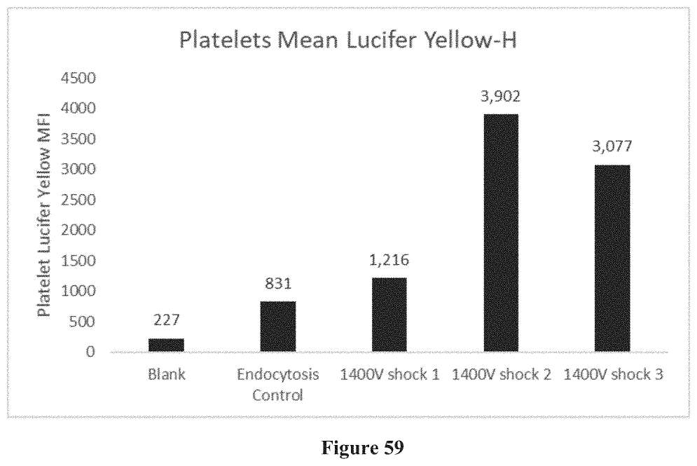

FIG. 59 shows Lucifer Yellow fluorescence in platelets under various loading conditions: no Lucifer Yellow exposure, no electroporation for three hours, one shock, two shocks with a 30 minute rest in between, and three shocks with 30 minute rests between shock 1 and 2 and shock 2 and 3. Maximum Lucifer Yellow fluorophore loading was seen after two shocks at 1400 V.

### Example 25. mRNA-Loaded Platelets

Examples of diseases (therapeutic indications) that may be treated with the RNA agent-loaded platelets are as follows:

Therapeutic indications
Acute lymphoblastic leukemia (ALL)
Acute myeloid leukemia (AML)
Breast cancer (can also be used as an adjuvant
therapy for metastasized breast cancer post-surgery)
Gastric cancer
Hodgkin lymphoma
Neuroblastoma
Non-Hodgkin lymphoma
Ovarian cancer
Cervical cancer
Small cell lung cancer
Non-small cell lung cancer (NSCLC)
Soft tissue and bone sarcomas
Thyroid cancer
Transitional cell bladder cancer
Wilms tumor
Neuroendocrine tumors
Pancreatic cancer
Multiple myeloma
Renal cancer
Glioblastoma
Prostate cancer
Sarcoma
Colon cancer
Melanoma
Colitis
Chronic inflammatory demyelinating polyneuropathy
Guillain-Barre syndrome
Immune Thrombocytopenia
Kawasaki disease
Lupus
Multiple Sclerosis
Myasthenia gravis
Myositis
Cirrhosis with refractory ascites
Hepatorenal syndrome (used in combination with
vasoconstrictive drugs)
Nephrotic syndrome (for patient with albumin
<2 g/dL with hypovolaemia and/or pulmonary edema)
Organ transplantation
Paracentesis
Hypovolemia
Aneurysms
Artherosclerosis
Cancer
Cardiovascular diseases (post-myocardial infarction
remodeling, cardiac regeneration, cardiac fibrosis,
viral myocarditis, cardiac hypertrophy, pathological
cardiac remodeling)
Genetic disorders
Infectious diseases
Metabolic diseases
Neoangiogenesis
Opthalmic conditions (retinal angiogenesis, ocular
hypertension, glaucoma, diabetic macular edema,
diabetic retinopathy, macular degeneration)
Hypercholesterolemia
Pulmonary hypertension

Examples of mRNA agent and therapeutic indications for mRNA agent(s) to be loaded into platelets (e.g., translated mRNA agents into their respective proteins) are as follows:

Fresh platelets were tested for their ability to uptake mRNA via lipofectamine transfection with Lipofectamine™ MessengerMAX™ (ThermoFisher Cat. #LMRNA003) with varying concentrations of suspended platelets and lipofectamine. A total of nine mRNA loaded platelet samples were prepared including 3 different platelet suspension concentrations (4 k/μl, 40 k/μl, and 100 k/μl) with 3 different concentrations of Lipofectamine™ MessengerMAX™ (0.0 μl, 0.75 μl, and 1.5 μl, each in 25 μl HBS) as described in Protocol 7. Each of the nine samples also contained 0.25 μg of CleanCAP Cyanine 5 EGFP mRNA (TriLink Cat. #L-7701; 1 mg/ml) also described in Protocol 7. All nine samples were assayed by flow cytometry for single platelet size (FSC/SSC), CY5 mean fluorescence intensity, and CY5 positive platelets, and absolute count at 30 minutes (Table 22) and at 120 minutes (Table 23).

TABLE 21
Loading conditions

| All 0.25 μg | 4 k/μL, | 40k/μL, | 100 k/μL |
|---|---|---|---|
| mRNA, | (Platelets) | (Platelets) | (Platelets) |
| 0.5 mL | 20x | 200x | 500x |
| volume | dilution | dilution | dilution |
| 0.75 μl | A | B | C |
| Lipofectamine | | | |
| 1.50 μl | D | E | F |
| Lipofectamine | | | |
| No Transfection | G | H | I |
| (HBS only) | | | |

TABLE 22
30 Minutes Transfection

| | | Platelet | Platelet | Cy5 |
|---|---|---|---|---|
| | Platelets | Size | Cy5 | Positive |
| Sample | per μL | (FSC-H) | MFI | Platelets |
| A | 3,520 | 248,128 | 7,781 | 68.70% |
| B | 33,893 | 283,856 | 3,561 | 35.27% |
| C | 91,741 | 287,321 | 1,444 | 20.20% |
| D | 3,855 | 284,302 | 5,131 | 99.32% |
| E | 32,424 | 282,348 | 2,620 | 62.96% |
| F | 85,964 | 283,569 | 1,063 | 33.94% |
| G | 5,615 | 267,719 | 279 | 0.33% |
| H | 38,377 | 274,645 | 313 | 0.92% |
| I | 89,701 | 276,510 | 318 | 0.99% |

TABLE 23
120 Minutes Transfection

| | Platelets | Platelet Size | Platelet Cy5 | Cy5 Positive |
|---|---|---|---|---|
| Sample | per μL | (FSC-H) | MFI | Platelets |
| A | 2,993 | 222,212 | 14,365 | 84.27% |
| B | 33,563 | 268,001 | 3,910 | 40.60% |
| C | 87,586 | 274,128 | 1,525 | 25.48% |
| D | 2,893 | 210,797 | 13,604 | 99.77% |
| E | 29,564 | 280,217 | 3,097 | 69.43% |
| F | 83,637 | 273,206 | 1,085 | 38.40% |
| G | 4,243 | 256,313 | 278 | 0.34% |
| H | 39,540 | 272,678 | 316 | 0.93% |
| I | 92,597 | 274,686 | 313 | 0.89% |

TABLE 24
HBS:

| | Concentration |
|---|---|
| Component | (mg/mL) |
| NaCl | 8.77 |
| HEPES | 2.38 |
| pH | 6.6-6.8 |

TABLE 25
Loading Buffer:

| | Concentration |
|---|---|
| | (mg/mL, except where |
| Component | otherwise indicated) |
| NaCl | 4.38 |
| KCl | 0.36 |
| HEPES | 2.26 |
| NaHCO₃ | 1.01 |
| Dextrose | 0.54 |
| Trehalose | 34.23 |
| Ethanol | 1.00% (v/v) |
| pH | 6.6-6.8 |

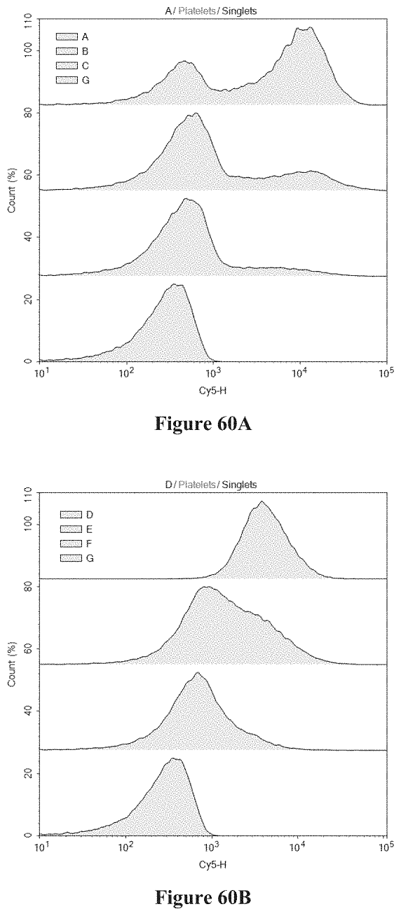

FIG. 60A and FIG. 60B are flow cytometry histograms showing detection of Cy5-H after 30 minutes of incubation for samples A through I shown in Table 21 indicating that platelets can be transfected with mRNA (CleanCAP Cyanine 5 EGFP mRNA) and lipofectamine transfection reagents, Lipofectamine™ MessengerMAX™.

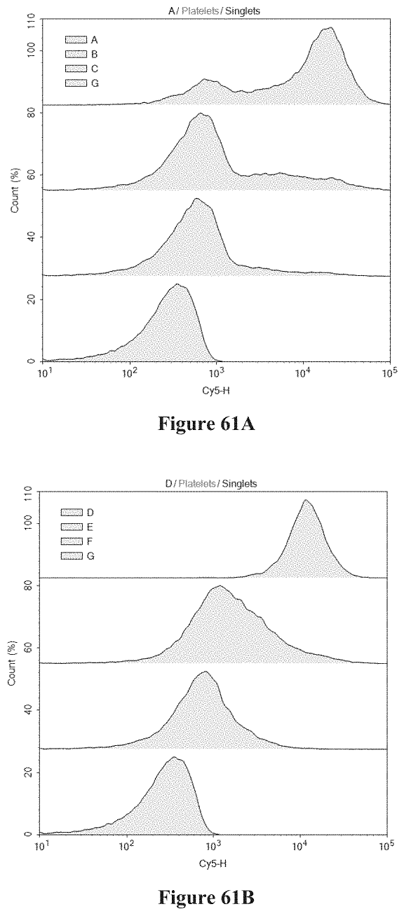

Cy5-H was detected after 120 minutes of incubation for samples A through I shown in Table 21 indicating that platelets can be transfected with mRNA with lipofectamine transfection reagents, Lipofectamine™ MessengerMAX™ (FIG. 61A and FIG. 61B).

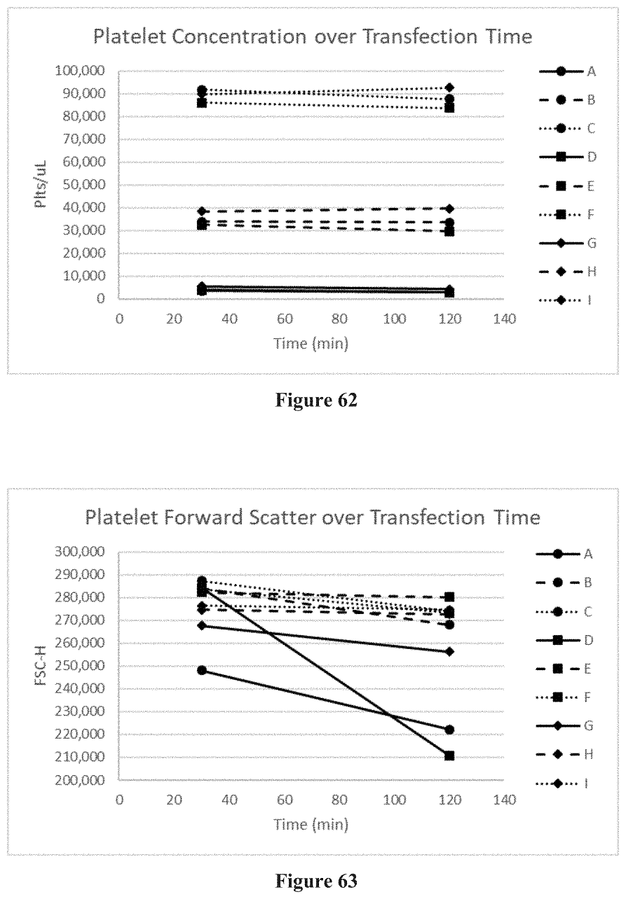

mRNA loaded platelet concentration (Plts/μl) was measured over 120 minutes of incubation time at 30 minutes and 120 minutes (FIG. 62). The three platelet suspension dilutions (4 k/μl, 40 k/μl, and 100 k/μl) transfected with varying concentrations of Lipofectamine™ MessengerMAX™ show no effect on platelet count after 120 minutes of incubation.

Platelet forward scatter (FSC-H) was measured over 120 minutes of an incubation time at 30 minutes and 120 minutes (FIG. 63). For most samples tested FSC-H decreases between the 30 minute measurement and the 120 minute measurement. For samples with low platelet concentration (4 k/μl) and a high lipofectamine concentration (0.75 μl, 1.5 μl), FSC-H decreased significantly (Samples A and D in Table 21).

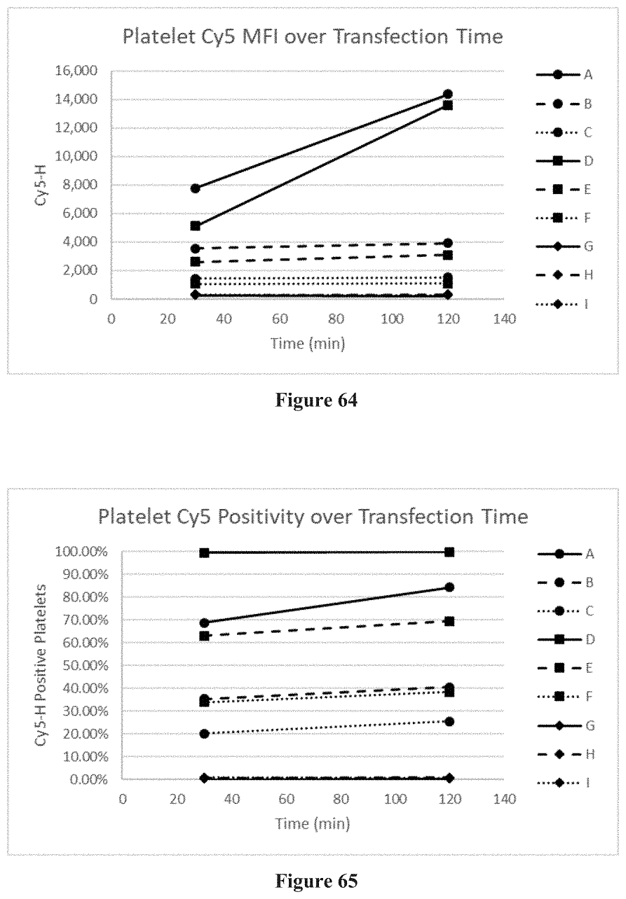

Cy5 mean fluorescent intensity (MFI) was measured over 120 minutes of an incubation time at 30 minutes and 120 minutes (FIG. 64). For samples tested with high platelet concentrations, 40 k/μl and 100 k/μl, Cy5 MFI was stable through two hours of incubation time. For tested samples with the lowest platelet concentration, 4 k/μl, Cy5 MFI increased from 30 minutes to 120 minutes incubation. All flow cytometry was performed with the Acea NovoCyte® (configuration 3005) flow cytometer for all flow data described herein.

Platelet Cy5-H positivity was measured over 120 minutes of an incubation time at 30 minutes and 120 minutes (FIG. 65). For all samples tested Cy5 positivity is stable beyond 30 minutes and showed slight increases, except sample A, in platelets over transfection time.

Protocol 7: Loading platelets with mRNA

The starting apheresis platelet material were washed in loading buffer via centrifugation to generate a master platelet stock at 1,000 platelets/μl (minimum 1 mL) in loading buffer. The washed platelets were diluted into three pools, 4 k/μL (samples A, D, and G), 40 k/μl (samples B, E, and H), and 100 k/μl (samples C, F, and I) (Table 21). Each sample contained 500 μl.

The lipofectamine transfection reagent, Lipofectamine™ MessengerMAX™ (ThermoFisher Cat. #LMRNA003) was prepared in 6 tubes, each tube containing 25 μl HBS. The six tubes were divided into two groups: 3 tubes containing 0.75 μl Lipofectamine™ MessengerMAX™ and 3 tubes containing 1.50 μl Lipofectamine™ MessengerMAX™. All tubes were incubated for 10 minutes at room temperature.

150 μL of HBS including 1.5 μl (1.5 μg) of Cy5-linked EGFP mRNA (TriLink Cat. #L-7701; 1 mg/mL) was prepared and incubated for 10 minutes at room temperature.

25 μl of the 150 μL of HBS including 1.5 μl (1.5 μg) of Cy5-linked EGFP mRNA mixture were allocated to each of the 3 tubes containing 0.75 μl Lipofectamine™ MessengerMAX™ and each of the 3 tubes containing 1.50 μl Lipofectamine™ MessengerMAX™ for a total volume of 50 μl and a concentration of Cy5-linked EGFP mRNA of 0.25 μg. Each tube was mixed and incubated at room temperature for 5 minutes.

Each of the six tubes containing 25 μl of HBS including 1.5 μl (1.5 μg) of Cy5-linked EGFP mRNA and including one of the varying concentrations of 25 μl of Lipofectamine™ MessengerMAX™ (either at a concentration of 0.75 μl or 1.50 μl) were added to the designated platelet suspension for a final volume of 550 μl. Negative control, “no transfection,” samples were prepared by adding an equivalent volume, 50 μl, of HBS to each of the designated platelet suspensions 4 k/μL, 40 k/μl, and 100 k/μl.

The platelet suspensions and transfection reagents were incubated for at least 2 hours at 37° C. with protection from ambient light. Incubation was conducted in a 2.0 mL snap-top microcentrifuge tube.

Platelet samples were analyzed at two time points, 30 minutes and 120 minutes, by flow cytometry (e.g., NovoCyte Flow Cytometer) for FSC/SSC (forward scatter/side scatter), Cy5 label detection, and absolute count.

Designated platelet suspension samples at 4 k/μl were diluted 2×. Designated platelet suspension samples at 40 k/μl were diluted 20×. Designated platelet suspension samples at 100 k/μl were diluted 50×.

Platelet suspension sample were assessed by eye for aggregation events.

### Example 26: mRNA Loaded Platelets

Fresh platelets were tested for their ability to uptake mRNA with varying concentrations of lipofectamine Lipofectamine™ MessengerMAX™ (ThermoFisher Cat. #LMRNA003) and varying concentrations of CleanCAP Cyanine 5 EGFP mRNA (TriLink L-7701; 1 mg/mL). All samples tested contained a suspended platelet dilution of about 40 k/μl in 500 μl in loading buffer.

Nine experimental conditions were tested with three concentrations of CleanCAP Cyanine 5 EGFP mRNA (0.25 μg, 0.50 μg, and 0.85 μg) and three concentrations of Lipofectamine™ MessengerMAX™ (1.50 μl, 3.00 μl, and 5.00 μl). The CleanCAP Cyanine 5 EGFP mRNA and Lipofectamine™ MessengerMAX mixtures were prepared according to Protocol 8. All nine samples were assayed by flow cytometry for single platelet size (FSC/SSC), CY5 mean fluorescent intensity, FITC, and absolute count at 30 minutes (Table 27) and 120 minutes (Table 28). A control sample containing no CleanCAP Cyanine 5 EGFP mRNA was included as well (Table26. Sample J).

TABLE 26
Loading conditions

| All ~40 k/μl | | | |
|---|---|---|---|
| plts, | 1.50 μl | 3.00 μl | 5.00 μl |
| 0.5 mL volume | Lipofectamine | Lipofectamine | Lipofectamine |
| 0.25 μl | A | B | C |
| mRNA | | | |
| 0.50 μg | D | E | F |
| mRNA | | | |
| 0.85 μl | G | H | I |
| mRNA | | | |
| No | J | N/A | N/A |
| Transfection | | | |
| Control | | | |
| (HBS only) | | | |

TABLE 27
30 Minutes Transfection

| | Platelets | Platelet Size | Platelet | Cy5 Positive |
|---|---|---|---|---|
| Sample | per μL | (FSC-H) | Cy5 MFI | Platelets |
| A | 35,767 | 245,993 | 1,465 | 73.45% |
| B | 27,419 | 300,997 | 3,572 | 99.00% |
| C | 21,201 | 282,855 | 3,919 | 98.45% |
| D | 32,539 | 252,273 | 2,303 | 24.96% |
| E | 31,145 | 270,048 | 3,930 | 96.48% |
| F | 18,989 | 307,676 | 5,307 | 99.48% |
| G | 29,827 | 232,654 | 1,799 | 68.71% |
| H | 26,943 | 252,389 | 4,563 | 31.85% |
| I | 23,416 | 268,113 | 5,733 | 81.64% |
| J | 28,991 | 243,790 | 256 | 0.96% |

TABLE 28
120 Minutes Transfection

| | Platelets | Platelet Size | Platelet | Cy5 Positive |
|---|---|---|---|---|
| Sample | per μL | (FSC-H) | Cy5 MFI | Platelets |
| A | 33,720 | 238,126 | 1,656 | 78.62% |
| B | 17,703 | 277,375 | 5,705 | 99.40% |
| C | 12,863 | 175,295 | 7,720 | 99.70% |
| D | 29,564 | 237,394 | 2,891 | 35.39% |
| E | 20,747 | 257,967 | 6,141 | 96.87% |
| F | 12,320 | 210,576 | 12,153 | 99.77% |
| G | 29,658 | 225,011 | 3,621 | 88.12% |
| H | 25,442 | 239,226 | 6,298 | 37.56% |
| I | 15,946 | 251,571 | 11,554 | 88.66% |
| J | 26,755 | 229,117 | 248 | 0.65% |

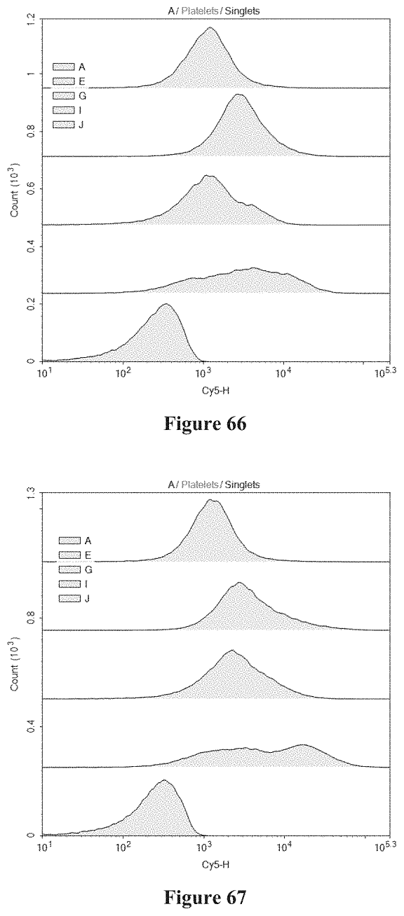

A flow cytometry histogram showing detection of Cy5-H after 30 minutes of incubation for samples A, E, G, I, and J (negative control) in Table 26 indicates that platelets can be transfected with mRNA (FIG. 66). Additionally, a flow cytometry histogram showing detection of Cy5-H after 120 minutes of incubation for samples A, E, G, I, and J (negative control) in Table 24 indicates that platelets can be transfected with mRNA (FIG. 67).

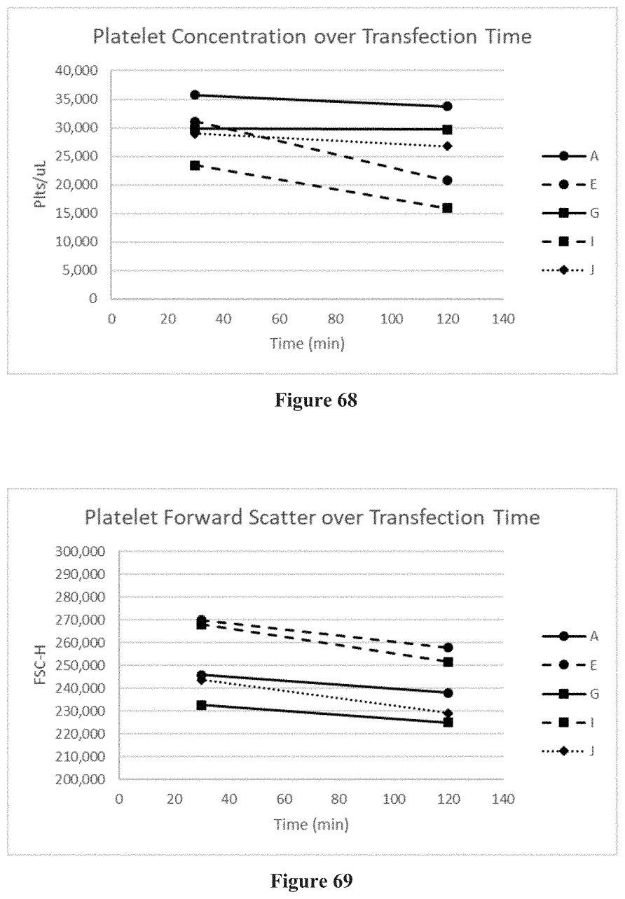

Single platelet count was measured over 120 minutes of incubation time at 30 minutes and 120 minutes for samples A, E, G, I, and J (negative control) in Table 26. Samples A, E, and J showed slight decreases in single platelet count, while samples E and I showed significant decreases in platelet count between measured time points 30 minutes and 120 minutes (FIG. 68).

Single platelet FSC-H was measured over 120 minutes of incubation time at 30 minutes and 120 minutes for samples A, E, G, I, and J (negative control) in Table 26. All tested samples showed a decrease in single platelet FSC-H between measured time points 30 minutes and 120 minutes, respectively (FIG. 69).

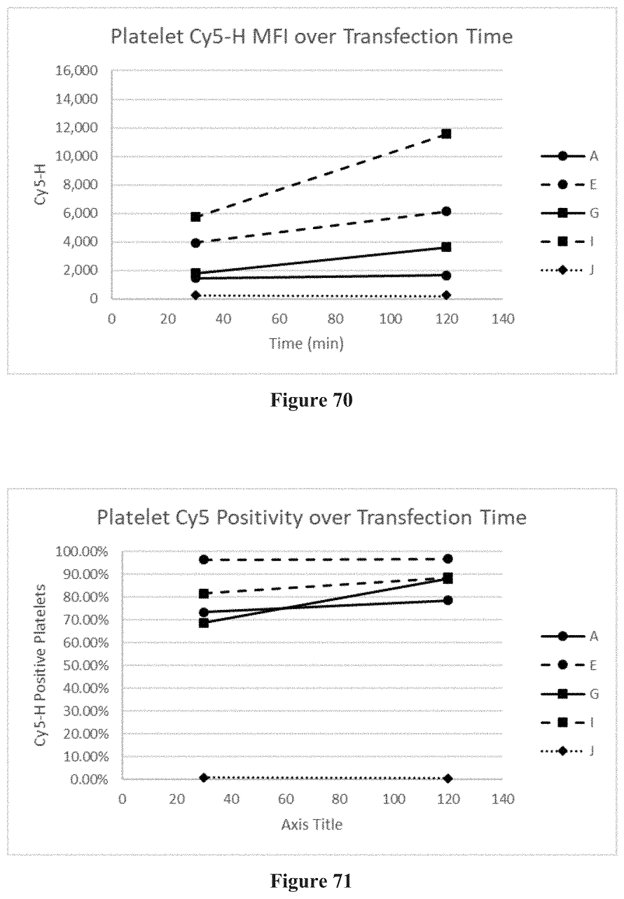

Singlet mRNA loaded platelet Cy5-H was measured over 120 minutes of incubation time at 30 minutes and 120 minutes for samples A, E, G, I, and J (negative control) in Table 26. Samples A, E, and G showed slight increases in mRNA loaded platelet Cy5-H. Sample I showed a significant increase in platelet Cy5-H measured between measured time points 30 minutes and 120 minutes, respectively (FIG. 70).

Singlet mRNA loaded platelet Cy5 positivity was measured over 120 minutes of incubation time at 30 minutes and 120 minutes for samples A, E, G, and I in Table 26. Samples A, E, and I showed slight increases in Cy5 positivity. Sample G showed a significant increase between measured time points 30 minutes and 120 minutes, respectively (FIG. 71).

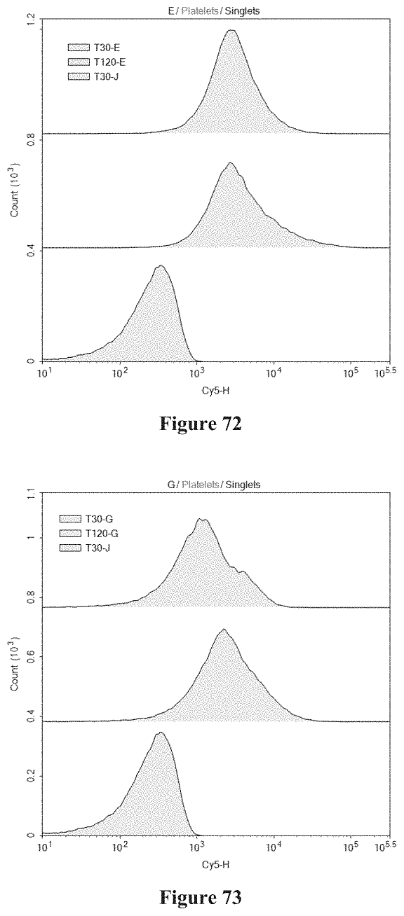

A flow cytometry histogram showing detection of Cy5-H in sample E from Table 26 after 30 minutes and 120 minutes of incubation time. Sample J (negative control) is also shown (bottom most peak) (FIG. 72).

A flow cytometry histogram showing detection of Cy5-H in sample G from Table 24 after 30 minutes and 120 minutes of incubation time. Sample J (negative control) is also shown (bottom most peak) (FIG. 73).

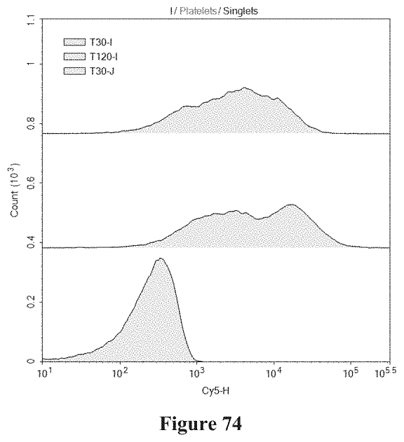

A flow cytometry histogram showing detection of Cy5-H in sample I from Table 24 after 30 minutes and 120 minutes of incubation time. Sample J (negative control) is also shown (bottom most peak) (FIG. 74).

Additionally, in accordance with Protocol 8, all test samples were checked by eye for platelet aggregation. No platelet aggregates were visible by eye at any timepoint.

Without being limited by any theory, the results from Sample G indicate that there may be an upper threshold to the quantity of mRNA that can be loaded into platelets. Additionally, Sample C and Sample F had apparent high toxicity, based on platelet count and FSC (data not shown) at 30 minutes. Further, Sample I demonstrated similar high toxicity at 120 minutes of incubation (FIGS. 68 and 69).

Test samples D and H demonstrated poor mRNA uptake with only a small proportion of the platelet population testing positive for Cy5 mRNA (Tables 27 and 28), while after 120 minutes of incubation Sample B had a dramatically reduced platelet count and the FSC had deteriorated significantly (Tables 27 and 28).

Protocol 8: Loading platelets with mRNA

The starting apheresis platelet material were washed in loading buffer via centrifugation to generate a master platelet stock at >1,000,000 platelets/μl (minimum 1 mL) in loading buffer described herein. The washed platelets were further diluted to a concentration of 50,000 platelets/μl using AcT counts as a guide. 500 μl were aliquoted for each test condition into a 2.0 mL snap top microcentrifuge tube.

The lipofectamine transfection reagent, Lipofectamine™ MessengerMAX™ (ThermoFisher Cat. #LMRNA003) was prepared in 9 tubes, each containing 25 μl total volume HBS as described herein. The 9 tubes were divided into three groups: 3 tubes containing 1.50 μl Lipofectamine MessengerMAX™, 3 tubes containing 3.0 μl of Lipofectamine MessengerMAX™, and 3 tubes containing 5.0 μl of Lipofectamine MessengerMAX™. All tubes were incubated at room temperature for 10 minutes.

Separately, Cy5-linked EFGP mRNA ((TriLink L-7701; 1 mg/mL) was added to 3 tubes containing 75 μl of HBS. One tube received 0.75 μl (0.75 ug) of Cy5-linked EGFP mRNA, one tube received 1.50 μl (1.50 μg) of Cy5-linked EGFP mRNA, and one tube received 2.55 μl (2.55 μg) of Cy5-linked EGFP mRNA. All tubes were mixed and incubated at room temperature for 10 minutes.

25 μl of each Cy5-linked EGFP mRNA concentration in 75 μl HBS were added to each of the concentrations of tubes containing the varying concentrations of Lipofectamine MessengerMAX™. For example, three aliquots of 25 μl of the 0.75 μl (0.75 μg) containing Cy5-linked EGFP mRNA were added to one tube each of the Lipofectamine MessengerMAX™ containing 1.5 μL, 3.0 μl, and 5.0 μl in HBS, respectively. Each tube was mixed and incubated at room temperature for 5 minutes.

Each of the nine tubes containing 25 μl of HBS including one of the varying concentrations of Cy5-linked EGFP mRNA (0.75 μg. 1.5 μg, and 2.55 μg, respectively) and 25 μl in HBS including one of the varying concentrations of Lipofectamine™ MessengerMAX™ (1.50 μl, 3.0 μl, and 5.0 μl, respectively) were added to 500 μl of diluted platelets at about 40 k platelets/μl for a final volume of 550 μl. Negative control, “no transfection,” samples were prepared by adding an equivalent volume, 50 μl, of HBS to each of the test conditions without the Lipofectamine™ MessengerMAX™ transfection reagent.

The platelet suspensions and transfection reagents were incubated for at least 2 hours at 37° C. with protection from ambient light. Incubation was conducted in a 2.0 mL snap-top microcentrifuge tube.

Platelet samples were analyzed at two time points, 30 minutes and 120 minutes, by flow cytometry (e.g., NovoCyte Flow Cytometer) for FSC/SSC (forward scatter/side scatter), Cy5 label detection, FITC, and absolute count after 20×dilution in HBS.

Platelet suspension samples were assessed by eye for aggregation events.

The disclosed embodiments, examples and experiments are not intended to limit the scope of the disclosure or to represent that the experiments below are all or the only experiments performed. Efforts have been made to ensure accuracy with respect to numbers used (e.g., amounts, temperature, etc.) but some experimental errors and deviations should be accounted for. It should be understood that variations in the methods as described may be made without changing the fundamental aspects that the experiments are meant to illustrate.

Those skilled in the art can devise many modifications and other embodiments within the scope and spirit of the present disclosure. Indeed, variations in the materials, methods, drawings, experiments, examples, and embodiments described may be made by skilled artisans without changing the fundamental aspects of the present disclosure. Any of the disclosed embodiments can be used in combination with any other disclosed embodiment.

In some instances, some concepts have been described with reference to specific embodiments. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of invention.

While the embodiments of the invention are amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

What is claimed is:

1. A method of preparing drug-loaded platelets, comprising:
treating platelets with a drug and with a loading buffer comprising a salt, a base, a loading agent, and optionally at least one organic solvent,
to form the drug-loaded platelets.

2. A method of preparing drug-loaded platelets, comprising:
c) providing platelets; and
d) treating the platelets with a drug and with a loading buffer comprising a salt, a base, a loading agent, and optionally at least one organic solvent
to form the drug-loaded platelets.

3. The method of any one of the preceding claims, wherein the platelets are treated with the drug and with the buffer sequentially, in either order.

4. A method of preparing drug-loaded platelets, comprising:
(3) treating platelets with a drug to form a first composition; and
(4) treating the first composition with a buffer comprising a salt, a base, a loading agent, and optionally at least one organic solvent, to form the drug-loaded platelets.

5. A method of preparing drug-loaded platelets, comprising:
(3) treating the platelets with a buffer comprising a salt, a base, a loading agent, and optionally at least one organic solvent to form a first composition; and
(4) treating the first composition with a drug, to form the drug-loaded platelets.

6. The method of claim 1 or 2, wherein the platelets are treated with the drug and with the buffer concurrently.

7. A method of preparing drug-loaded platelets, comprising:
treating the platelets with a drug in the presence of a buffer comprising a salt, a base, a loading agent, and optionally at least one organic solvent to form the drug-loaded platelets.

8. The method of any one of the preceding claims, wherein the platelets are pooled from a plurality of donors prior to a treating step.

9. A method of preparing drug-loaded platelets comprising
C) pooling platelets from a plurality of donors; and
D) treating the platelets from step (A) with a drug and with a loading buffer comprising a salt, a base, a loading agent, and optionally at least one organic solvent, to form the drug-loaded platelets.

10. A method of preparing drug-loaded platelets comprising
C) pooling platelets from a plurality of donors; and
D)
(1) treating the platelets from step (A) with a drug to form a first composition; and
(2) treating the first composition with a buffer comprising a salt, a base, a loading agent, and optionally at least one organic solvent, to form the drug-loaded platelets.

11. A method of preparing drug-loaded platelets comprising
C) pooling platelets from a plurality of donors; and
D)
(1) treating the platelets from step (A) with a buffer comprising a salt, a base, a loading agent, and optionally at least one organic solvent, to form a first composition; and
(2) treating the first composition with a drug to form the drug-loaded platelets.

12. A method of preparing drug-loaded platelets comprising
C) pooling platelets from a plurality of donors; and
D) treating the platelets with a drug in the presence of a buffer comprising a salt, a base, a loading agent, and optionally at least one organic solvent, to form the drug-loaded platelets.

13. The method of any one of the preceding claims, wherein the loading agent is a monosaccharide or a disaccharide.

14. The method of any one of the preceding claims, wherein the loading agent is sucrose, maltose, trehalose, glucose, mannose, or xylose.

15. The method of any one of the preceding claims, wherein the platelets are isolated prior to a treating step.

16. The method of any one of the preceding claims, wherein the platelets are loaded with the drug in a period of time of 5 minutes to 48 hours.

17. The method of any one of the preceding claims, wherein the concentration of drug in the drug-loaded platelets is from about 1 nM to about 100 mM.

18. The method of any one of the preceding claims, wherein the one or more organic solvents selected from the group consisting of ethanol, acetic acid, acetone, acetonitrile, dimethylformamide, dimethyl sulfoxide, dioxane, methanol, n-propanol, isopropanol, tetrahydrofuran (THF), N-methyl pyrrolidone, dimethylacetamide (DMAC), or combinations thereof.

19. The method of any one of the preceding claims, further comprising cold storing, cryopreserving, freeze-drying, thawing, rehydrating, and combinations thereof the drug-loaded platelets.

20. The method of claim 19, wherein the drying step comprises freeze-drying the drug-loaded platelets.

21. The method of claim 19 or 20, further comprising rehydrating the drug-loaded platelets obtained from the drying step.

22. Drug-loaded platelets prepared by the method of any one of the preceding claims.

23. Rehydrated drug-loaded platelets prepared by a method comprising rehydrating the drug-loaded platelets of claim 22.

24. The method of any one of the preceding claims, wherein the drug is modified with an imaging agent.

25. The method of claim 24, wherein the drug is modified with the imaging agent prior to treating platelets with the drug.

26. The method of any one of the preceding claims, wherein the platelets are further treated with an imaging agent, wherein the drug-loaded platelets are loaded with the imaging agent.

27. The method of any one of the preceding claims, wherein the method does not comprise treating the platelets with an organic solvent.

28. The method of any one of claims 4, 5, 10 or 11, wherein the method does not comprise treating the first composition with an organic solvent.

29. The method of any one of the preceding claims, wherein the method comprises treating the platelets with Prostaglandin E1 (PGE1) or Prostacyclin.

30. The method of any one of claims 1 to 28, wherein the method does not comprise treating the platelets with Prostaglandin E1 (PGE1) or Prostacyclin.

31. The method of any one of the preceding claims, wherein the method comprises treating the platelets with a chelating agent such as EGTA.

32. The method of any one of claims 1 to 30, wherein the method does not comprises treating the platelets with a chelating agent such as EGTA.

33. The method of any one of claims 1 to 29, wherein the method comprises treating the first composition with Prostaglandin E1 (PGE1) or Prostacyclin.

34. The method of any one of claims 1 to 28 or 30, wherein the method does not comprise treating the first composition with Prostaglandin E1 (PGE1) or Prostacyclin.

35. The method of any one of claims 1 to 31, 33 or 34, wherein the method comprises treating the first composition with a chelating agent such as EGTA.

36. The method of any one of claims 1 to 30 or 32 to 34, wherein the method does not comprises treating the first composition with a chelating agent such as EGTA.

37. The method of any one of the preceding claims, wherein the method further comprises treating the drug-loaded platelets with an anti-aggregation agent.

38. The method of claim 37, wherein the anti-aggregation agent is a GPIIb/IIIa inhibitor.

39. The method of claim 38, wherein the GPIIb/IIIa inhibitor is GR144053.

40. The method of claim 39, wherein GR144053 is present in a concentration of at least 1.2 μM.

41. The method of any one of claims 38-41, wherein the platelets are treated with the anti-aggregation agent before being treated with the drug.

42. The method of any one of claims 38-41, wherein the platelets are treated with the anti-aggregation agent concurrently with the drug.

43. The method of any one of the preceding claims, wherein the drug is a small molecule, a protein, an oligopeptide, an aptamer, and combinations thereof.

44. The method of any one of the preceding claims, wherein the drug is a drug for the treatment of cancer.

45. The method of claim 44, wherein the cancer comprises hemangiosarcoma.

46. The method of claims 44-45, wherein the drug for the treatment of cancer is doxorubicin.

47. The method of claims 45-46, wherein the drug for the treatment of hemangiosarcoma is doxorubicin.

48. The method of claim 44, wherein the drug for the treatment of cancer is paclitaxel.

49. The method of claim 44, wherein the drug for the treatment of cancer is a PARP inhibitor.

50. The method of claim 49, wherein the PARP inhibitor is olaparib.
